(19) United States
(12) Patent Application Publication
(10) Pub. No.: US 2026/0345042 A1
(43) Pub. Date: Nov. 5, 2026

(54) BIOMETRIC ANALYSIS SYSTEM AND METHOD FOR ASSESSING PHYSIOLOGICAL AND EMOTIONAL STATES

(71) Applicant: Michella Metzler, New York, NY (US)

(72) Inventor: Michella Metzler, New York, NY (US)

(21) Appl. No.: 19/659,062

(22) Filed: Apr. 27, 2026

Related U.S. Application Data
(60) Provisional application No. 63/797,057, filed on Apr. 29, 2025.

(51) Int. Cl. A61B 5/16; A61B 5/00; A61B 5/0205

(52) U.S. Cl. CPC A61B 5/165; A61B 5/02055; A61B 5/4836; A61B 5/7267; A61B 5/7275

(57) ABSTRACT

A biometric analysis system comprises one or more diagnostic sensors, and at least one processor configured to receive the biological data and extract signal characteristics comprising frequency-domain features and waveform-based features; convert, the extracted signal characteristics into digital representations; correlate, the somatic imprints with emotional, neurological, and behavioral states of the user, and generate a quantified emotional load profile; map, the physiological conditions and the quantified emotional load profile to corresponding reference parameters; evaluate, the physiological conditions and the quantified emotional load profile mapped to the corresponding reference parameters against reference data indicative of healthy and pathological states to determine system-level imbalances comprising at least one of inflammation, stress response, metabolic efficiency, and recovery capacity; generate, a personalized intervention plan based on the determined system-level imbalances; and iteratively update, the personalized intervention plan based on subsequently acquired biological data to enable continuous recalibration of the physiological and emotional state.

### CROSS REFERENCE

This application is a US Non-Provisional Patent Application claims the priority benefit from U.S. Provisional Patent Application No. 63/797,057 filed on Apr. 29, 2025, which is incorporated by reference herein in its entirety.

### FIELD OF THE DISCLOSURE

The present invention relates in general to a biometric system, and particularly relates to a biometric analysis system and a method for assessing physiological states of human.

### BACKGROUND OF THE DISCLOSURE

Evaluation of human health has traditionally relied on a range of diagnostic methods, including clinical bloodwork, imaging technologies such as CT scans, and other biochemical or mechanical testing tools. While these approaches are well-established, they typically depend on analog systems, direct physical interaction, and often require invasive procedures or time-consuming laboratory analysis. Such limitations can delay diagnosis, restrict continuous monitoring, and overlook subtle physiological and emotional imprints that may be relevant in preventive or integrative healthcare. There is increasing interest in diagnostic platforms capable of detecting and interpreting physiological, energetic, and emotional information using alternative modalities. Such systems would ideally enable observation of biological changes through non-invasive means, without the need for needles, imaging contrast agents, or physical samples, by analyzing subtle signals generated by the body at a bioelectrical, quantum, or frequency level.

Several emerging approaches attempt to utilize wave-based technologies and signal processing tools to assess the functional status of tissues, organs, or biological systems. These approaches include, for example, resonance analysis, frequency pattern recognition, and electromagnetic field detection. However, existing solutions in this domain often lack the accuracy, integration, or adaptability required for comprehensive human body assessment, particularly with respect to linking biological activity with emotional or psychosomatic indicators. Accordingly, there remains a need for a diagnostic system that does not rely on traditional analog methods, but instead utilizes advanced sensing technologies, wave-based input/output mechanisms, and intelligent software analysis to investigate the condition of the human body. Preferably, such a system would operate on a non-contact basis, interpret wave characteristics associated with biological structures, and provide digital data that can be visualized, interpreted, and used for personalized diagnostics and health planning.

Recent advances in bioelectric medicine and psychophysiology have demonstrated that biological tissues generate measurable electromagnetic and biofrequency signals that reflect underlying physiological and regulatory states. Cellular membrane potentials, electrochemical signaling, and tissue-level bioelectric fields dynamically vary in response to metabolic activity, inflammatory conditions, and external stressors. These bioelectric characteristics propagate across interconnected tissue networks, thereby enabling system-level assessment of physiological conditions through non-invasive signal acquisition and analysis. Further, emerging research indicates that emotional and psychological states are not solely neurological phenomena but are physiologically encoded within the body. Chronic emotional stress produces measurable alterations in autonomic, endocrine, and immune responses, resulting in persistent changes in tissue-level electrical and biochemical activity. These alterations manifest as localized and systemic patterns of dysregulation that can be detected through biofrequency-based measurement techniques, thereby providing an objective basis for identifying emotional and neurological influences on physiological function.

The fascial system, comprising a continuous network of connective tissue throughout the body, has been identified as a medium for such physiological encoding. Due to its piezoelectric properties, fascial tissue generates electrical signals in response to mechanical stress and deformation. Chronic emotional or physical stress can result in sustained fascial restriction, leading to localized electrical deviations that serve as indicators of somatic encoding. These measurable deviations provide spatially specific information regarding the location and intensity of physiological and emotional dysregulation within the body. Additionally, the autonomic nervous system plays a central role in mediating the relationship between emotional states and physiological responses. Persistent activation of sympathetic or parasympathetic pathways produces characteristic patterns of biofrequency variation across multiple organ systems. These patterns correspond to distinct regulatory states, including stress activation, reduced recovery capacity, and impaired metabolic function, thereby enabling identification of system-level imbalances through analysis of biofrequency data.

Traditional diagnostic frameworks, including those derived from Traditional Chinese Medicine, have long identified correlations between specific organ systems and emotional states. Contemporary psychophysiological research increasingly supports these correlations, demonstrating that emotional patterns such as stress, anxiety, or grief are associated with measurable physiological changes in corresponding organ systems. However, existing diagnostic systems do not provide a unified mechanism for integrating biofrequency measurements, emotional correlations, and physiological assessment into a coherent analytical framework. Accordingly, there exists a need for improved systems capable of acquiring biological signals, extracting meaningful signal characteristics, and integrating physiological and emotional information to identify system-level imbalances. In particular, there is a need for systems that can interpret biofrequency data in a multi-dimensional manner, correlate physiological conditions with emotional and neurological states, and perform adaptive comparison with reference data indicative of healthy and pathological conditions. Such systems would enable more precise and context-aware assessment of biological function and provide a foundation for generating personalized intervention strategies.

Prior art, for various aspects relevant to this disclosure, includes US Patent Publication No. 20220379117A1 to Kumar, US Patent Publication No. 8298153B2 to Dunlap, and US Patent Publication No. 20230210470A1 to Stacy. These references describe diagnostic systems that utilize electromagnetic wave detection and frequency analysis for identifying health conditions. However, these systems typically rely on analog-based technology or invasive methods and do not incorporate a holistic, analogue-free, wave-based diagnostic system for non-contact, real-time monitoring of both physiological and emotional states, as described in the present invention. The existing systems also do not provide the unique ability to decode somatic imprints or quantified emotional load profile and other inferred physiological conditions based on wave characteristics from biological tissues, cells, and organs.

In particular, '117 to Kumar discloses a system that detects electromagnetic frequencies from biological tissues and uses them for diagnostic purposes. However, this system is limited in its ability to integrate artificial intelligence (AI) or quantum-based diagnostics to process the electromagnetic waves. Additionally, it does not utilize non-invasive sensors, nor does it correlate changes in tissue wave characteristics to emotional states or quantified emotional load profile and other inferred physiological conditions. Moreover, this system lacks the capacity to visualize or quantify the emotional and somatic imprint of organs in real time, and it fails to provide personalized recommendations for therapeutic actions based on the detected bioelectrical activity.

Reference, '153 to Dunlap describes a diagnostic tool for monitoring various physiological states using non-linear analysis of biological signals. Although the device is capable of identifying changes in tissue and organ functions, it does not employ the use of electromagnetic waves or quantum chromo kinetics for the detailed visualization of organ health. Furthermore, the system described does not support the use of a virtual model of the human body that can be dynamically updated based on frequency analysis. The ability to correlate entropy levels and somatic imprints with specific emotional states is not addressed by this prior art.

Reference, '470 to Stacy introduces a diagnostic platform that measures electromagnetic properties of biological tissues and provides feedback for health optimization. However, this system primarily focuses on physical diagnostics and lacks a method for integrating quantified emotional load profile and other inferred physiological conditions or psychological conditions into the analysis. It also does not provide a detailed, AI-assisted interface that decodes real-time wave characteristics and visualizes them in a color-coded, 3D models for easier understanding of the patient's health status. Moreover, the system does not include a telemetric nonlinear analysis apparatus that can decode faint signals from the biological source in a non-invasive manner.

Reference, '039 describes a system for real-time health monitoring using wearable devices that track common biomarkers such as heart rate, temperature, and blood oxygen levels. However, the system is based on conventional biometrics and does not utilize wave characteristics from biological tissues, cells, or organs. It lacks the capability to detect and interpret emotional states or to offer a personalized diagnostic report based on non-contact frequency analysis. The device also fails to incorporate AI or quantum diagnostic tools, limiting its potential for integrating emotional and somatic health analysis.

Reference, '288 relates to a diagnostic system that employs artificial intelligence to assist in interpreting biological signals for health monitoring. However, this reference does not disclose the use of advanced wave-based sensors for non-invasive diagnostics, nor does it provide a system for correlating detected biological signals with quantified emotional load profile and other inferred physiological conditions or psychological well-being. Additionally, the system lacks the ability to visualize the data in an intuitive, color-coded, 3D format that is based on quantum chromo kinetics principles, as is proposed in the present invention.

Reference, '896 outlines a diagnostic device that analyzes biological signals for detecting various conditions. However, this system is focused on specific conditions and does not provide the ability to analyze and display a broad spectrum of health indicators, including emotional and somatic states, using wave characteristics. The lack of integration between frequency analysis, AI-based data processing, and real-time quantified emotional load profile and other inferred physiological conditions quantification restricts its capability for holistic and personalized diagnostics. Furthermore, it does not include an intuitive user interface that can generate a virtual, color-coded model of the human body for easier interpretation of the results.

Given the limitations of the prior art, the need remains for a system that can provide a non-invasive, analogue-free diagnostic platform capable of detecting and decoding both physiological and emotional conditions in real-time. Unlike existing solutions, which are limited by analog systems or focus solely on isolated aspects of health diagnostics, the present invention offers a comprehensive, wave-based investigation system. This system integrates advanced sensors, quantum-level analysis, and artificial intelligence to dynamically process and visualize biological data. By utilizing frequency-based inputs and decoding somatic imprints, it enables a holistic approach to health assessment, offering real-time feedback on quantified emotional load profile and other inferred physiological conditions, organ health, and energy imbalances. Such a system has the potential to provide personalized diagnostics and therapeutic recommendations, fostering more effective, individualized healthcare solutions.

### SUMMARY OF THE DISCLOSURE

In accordance with the present disclosure, a biometric analysis system provides a unique, non-invasive, and analogue-free solution for real-time diagnostics that combines the power of electromagnetic wave detection, AI-based analysis, and quantum-level diagnostics. One of the primary advantages of the biometric analysis system is its ability to decode subtle biological signals from the body, providing comprehensive insights into both the physiological and emotional states of the patient. The integration of signal-based physiological analysis system with non-contact frequency analysis enables the detection of health imbalances that traditional diagnostic methods may overlook. By processing these signals through AI algorithms, the system can generate real-time 3D visualizations of the organs, providing doctors with a deeper understanding of a patient's health. The color-coded entropy visualization helps healthcare providers to easily identify areas of concern and offers a dynamic, personalized diagnostic approach that enhances patient care and health optimization strategies.

In one embodiment of the present disclosure, the biometric analysis system comprises one or more diagnostic sensors capable of being integrated into wearable devices, such as headphones or other portable platforms. These sensors transmit electromagnetic waves that recognize specific frequencies (Vortex magnetic fields) to detect functional imbalances in the body. The system can be used for various types of signal-based physiological analysis system, including biodecodification, bloodwork, hormone panel assessments, and DEXA scans all without requiring physical contact with the patient. By analyzing the emitted waves, the system decodes the body's bioelectrical activity, amplifies selective signals, and isolates the relevant data to determine the body's current functional status.

In one aspect of the present disclosure, the biometric analysis system decodes somatic imprints from the body, including emotional quantified emotional load profile and other inferred physiological conditions, by analyzing changes in the wave characteristics that emanate from tissues and organs. Further, the biometric analysis system comprises artificial intelligence (AI) modules that process this data and provide a quantitative analysis of a patient's emotional state and the corresponding physiological conditions. The biometric analysis system translates these findings into visual models, including 3D projections of internal organs, which are color-coded based on entropy values that range from healthy (light yellow) to pathological (red, purple, and nearly black). These visualizations allow healthcare providers to observe the emotional and somatic imprints of different organs in real-time, enhancing their diagnostic capabilities.

In another aspect of the present disclosure, the biometric analysis system is designed to provide real-time feedback to both the patient and physician. The biometric analysis system is capable of analyzing biological signals and comparing them to a database of known healthy and pathological frequencies to identify patterns indicative of potential physiological imbalance tendencies. This enables early detection of pathological conditions, as well as the monitoring of ongoing health optimization strategies. In addition, the biometric analysis system suggests personalized remedies, including health optimization and longevity strategies, by comparing detected frequencies to a database of remedy frequencies that correspond to specific health conditions.

In another aspect of the present disclosure, the biometric analysis system utilizes a user interface, which is designed to be intuitive and provides detailed data reports for the user. This user interface includes a main menu with features such as patient reception, settings, and access to individual patient cards containing personal information. The biometric analysis system also includes a “Research” mode, where users may perform in-depth, nonlinear analysis of specific organs, detect functional disturbances, and visualize localized regions of physiological or functional deviations within one or more organs or biological systems. The AI-based modules process the data, providing a color-coded 3D projection of the affected organs, highlighting areas with pathological changes in varying shades from green (healthy) to brown and red (dysfunctional).

In another aspect of the present disclosure, the biometric analysis system allows for dynamic updates based on the patient's quantified emotional load profile and other inferred physiological conditions and other health conditions. For instance, the biometric analysis system by using the One or more AI models may quantify the patient's quantified emotional load profile and other inferred physiological conditions, a unique feature that distinguishes it from conventional diagnostic devices. The biometric analysis system also enables physicians to track the progress of health conditions over time and adjust treatment plans accordingly. Additionally, the biometric analysis system is able to suggest anti-aging treatments, preventive measures, and customized longevity strategies based on the detected health conditions and their emotional correlations.

In one aspect of the present invention, a biometric analysis system is disclosed. The biometric analysis system comprises one or more diagnostic sensors integrated into a device configured to be positioned in proximity to a user, the one or more diagnostic sensors are configured to transmit electromagnetic excitation signals within one or more predefined frequency ranges and receive corresponding electromagnetic response signals generated from interaction with the user to acquire biological data from the user, the biological data comprises at least bioelectrical activity, electromagnetic wave characteristics, biofrequency patterns, and physiological parameters associated with physiological activity of one or more tissues, one or more organs, and cellular activity; and at least one processor communicatively coupled to the one or more diagnostic sensors and a memory storing instructions, wherein the at least one processor, upon executing the instructions, is configured to receive the biological data derived from the electromagnetic response signals and extract signal characteristics comprises frequency-domain features and waveform-based features representative of functional states of the user using one or more signal transformation techniques; convert, via a decoding module communicatively coupled to the at least one processor, the extracted signal characteristics into digital representations representing inferred physiological conditions and somatic imprints representing inferred functional states of the one or more tissues, the one or more organs or biological systems based on the extracted signal characteristics; correlate, via a correlation module communicatively coupled to the at least one processor, the somatic imprints with emotional, neurological, and behavioral states of the user by applying one or more artificial intelligence (AI) models trained on reference data comprising physiological signal patterns and corresponding functional states, and generate a quantified emotional load profile computed from physiological signal variations corresponding to autonomic nervous system activity; map, via a mapping module communicatively coupled to the at least one processor, the physiological conditions and the quantified emotional load profile to corresponding reference parameters within the reference data indicative of healthy and pathological states, by applying the one or more AI models; evaluate, via an analysis engine communicatively coupled to the at least one processor, the physiological conditions and the quantified emotional load profile mapped to the corresponding reference parameters within the reference data to determine system-level imbalances; generate, via a protocol generation module communicatively coupled to the at least one processor, a personalized intervention plan based on the determined system-level imbalances; and iteratively update, via a feedback module communicatively coupled to the at least one processor, the personalized intervention plan based on subsequently acquired biological data to enable continuous recalibration of the physiological and emotional state of the user.

In another aspect of the present disclosure, the one or more diagnostic sensors comprise electromagnetic sensors configured to transmit and receive the electromagnetic excitation signals and the electromagnetic response signals to acquire the biofrequency patterns corresponding to the physiological activity of the one or more tissues or the one or more organs of the user, wherein the one or more diagnostic sensors comprise at least one of an electromagnetic transceiver, a biofrequency sensor, a resonance-based sensor, or a bioimpedance sensor.

In another aspect of the present disclosure, the frequency-domain features comprise spectral components and resonance characteristics of the biological data derived from the electromagnetic response signals, and the waveform-based features comprise amplitude, phase, and temporal variations of the biological data, the frequency-domain features and the waveform-based features being indicative of functional states of the user, and the one or more signal transformation techniques comprises at least spectral decomposition, resonance characteristic identification, and detection of resonance peaks, impedance variations, and frequency response signatures corresponding to the physiological activity of the one or more tissues or the one or more organs.

In another aspect of the present disclosure, the somatic imprints comprise data representations derived from the extracted signal characteristics that correspond to functional conditions of the physiological activity of the one or more tissues, the one or more organs, or the biological systems of the user, and the system level imbalances comprises at least one of inflammation, stress response, metabolic efficiency, and recovery capacity based on deviations in the extracted signal characteristics.

In another aspect of the present disclosure, the device is configured as a wearable or user-adjacent device, wherein the device comprises a head-mounted device comprises at least headphones, earphones, earbuds, a headset, a wearable band, a smartwatch, a handheld device, or a computing device configured to be positioned in proximity to the user.

In another aspect of the present disclosure, the personalized intervention plan comprises a multi-stage framework for modifying the physiological and emotional state of the user.

In another aspect of the present disclosure, the multi-stage framework comprises a reset phase configured to reduce inflammatory load and physiological interference signals, a replenish phase configured to provide personalized biochemical and nutritional support, a regulate phase configured to modify neural and emotional response patterns, and a performance phase configured to enhance physiological and cognitive function. The at least one processor enables the device to further generate a feedback signal comprising at least one electromagnetic or frequency-based output signal configured to be applied to the user, wherein the feedback signal is provided in accordance with the personalized intervention plan to modulate at least one physiological or neurological state of the user.

In another aspect of the present disclosure, the replenish phase comprises generating a personalized supplementation formulation including a combination of vitamins, minerals, botanicals, and nootropic compounds based on the system-level imbalances, wherein the personalized supplementation formulation comprises between 60 and 90 compounds.

In another aspect of the present disclosure, the regulate phase comprises one or more structured neural retraining sessions configured to modify subconscious emotional and behavioral patterns of the user, wherein the one or more structured neural retraining sessions comprise a predefined sequence of sessions conducted over a plurality of time intervals.

In another aspect of the present disclosure, the at least one processor using a user interface operatively coupled to the at least one processor, is further configured to present the physiological conditions, the quantified emotional load profile, and the personalized intervention plan, wherein the user interface is executed on at least one of the device or an external computing device communicatively coupled to the biometric analysis system.

In another aspect of the present disclosure, the at least one processor is further configured to present the physiological conditions and the quantified emotional load profile using a color spectrum corresponding to entropy values associated with the physiological conditions or the quantified emotional load profile, wherein the color spectrum comprises a gradient ranging from lighter colors representing lower entropy values to darker colors representing higher entropy values.

In another aspect of the present disclosure, the at least one processor using the user interface is further configured to present a three-dimensional representation of one or more organs or biological systems of the user, and to display one or more visual markers on the three-dimensional representation corresponding to locations of the physiological conditions or system-level imbalances.

In another aspect of the present disclosure, the analysis engine is further configured to compare spectral characteristics of the biological data with reference spectral data corresponding to healthy tissue and pathological conditions to determine a degree of similarity and identify a corresponding pathological tendency.

In another aspect of the present disclosure, the quantified emotional load profile comprises weighted indicators corresponding to stress, emotional reactivity, and behavioral patterns derived from the physiological signal variations.

In another aspect of the present disclosure, the analysis engine is configured to evaluate the physiological conditions and the quantified emotional load profile as a combined multi-domain model for determining the system-level imbalances.

In another aspect of the present disclosure, the one or more AI models are trained based on relationships between the physiological conditions, emotional load factors, and the reference parameters.

In one aspect of the present disclosure a biometric analysis device positioned in proximity to a user is disclosed. The biometric analysis device comprises one or more diagnostic sensors integrated into the biometric analysis device, wherein the one or more diagnostic sensors are configured to transmit electromagnetic excitation signals within one or more predefined frequency ranges and receive corresponding electromagnetic response signals generated from interaction with the user to acquire biological data from the user, the biological data comprising at least bioelectrical activity, electromagnetic wave characteristics, biofrequency patterns, and physiological parameters associated with physiological activity of one or more tissues, one or more organs, and cellular activity; and at least one processor communicatively coupled to the one or more diagnostic sensors and a memory storing instructions, wherein the at least one processor, upon executing the instructions, is configured to receive the biological data derived from the electromagnetic response signals and extract signal characteristics comprising frequency-domain features and waveform-based features representative of functional states of the user using signal transformation techniques; convert, via a decoding module communicatively coupled to the at least one processor, the extracted signal characteristics into digital representations representing inferred physiological conditions and somatic imprints representing inferred functional states of the one or more tissues, the one or more organs or biological systems based on the extracted signal characteristics; correlate, via a correlation module communicatively coupled to the at least one processor, the somatic imprints with emotional, neurological, and behavioral states of the user by applying one or more artificial intelligence (AI) models trained on reference data comprising physiological signal patterns and corresponding functional states, and generate a quantified emotional load profile computed from physiological signal variations corresponding to autonomic nervous system activities; map, via a mapping module communicatively coupled to the at least one processor, the physiological conditions and the quantified emotional load profile to corresponding reference parameters within the reference data indicative of healthy and pathological states, by applying the one or more AI models; evaluate, via an analysis engine communicatively coupled to the at least one processor, the physiological conditions and the quantified emotional load profile mapped to the corresponding reference parameters within the reference data to determine system-level imbalances; generate, via a protocol generation module communicatively coupled to the at least one processor, a personalized intervention plan based on the determined system-level imbalances; and iteratively update, via a feedback module communicatively coupled to the at least one processor, the personalized intervention plan based on subsequently acquired biological data to enable continuous recalibration of the physiological and emotional state of the user.

In one aspect of the present disclosure a computer-implemented method for biometric analysis is disclosed. The computer-implemented method comprises transmitting and receiving, via one or more diagnostic sensors integrated into a device configured to be positioned in proximity to a user, electromagnetic excitation signals to acquire biological data from the user, the biological data comprises at least bioelectrical activity, electromagnetic wave characteristics, biofrequency patterns, and physiological parameters associated with physiological activity of one or more tissues, one or more organs, and cellular activity; receiving, via at least one processor communicatively coupled to the one or more diagnostic sensors and a memory storing instructions, the biological data and extract signal characteristics comprises frequency-domain features and waveform-based features representative of functional states of the user; converting, via a decoding module communicatively coupled to the at least one processor, the extracted signal characteristics into digital representations representing inferred physiological conditions and somatic imprints representing inferred functional states of the one or more organs or biological systems based on the extracted signal characteristics; correlating, via a correlation module communicatively coupled to the at least one processor, the somatic imprints with emotional, neurological, and behavioral states of the user by applying one or more artificial intelligence (AI) models configured to infer relationships between physiological representations and emotional states, and generate a quantified emotional load profile based on the determined relationships; mapping, via a mapping module communicatively coupled to the at least one processor, the physiological conditions and the quantified emotional load profile to corresponding reference parameters within the reference data indicative of healthy and pathological states, by applying the one or more AI models configured to learn relationships between the physiological conditions, emotional load factors, and the reference parameters; evaluating, via an analysis engine communicatively coupled to the at least one processor, the physiological conditions and the quantified emotional load profile mapped to the corresponding reference parameters against reference data indicative of healthy and pathological states to determine system-level imbalances comprises at least one of inflammation, stress response, metabolic efficiency, and recovery capacity; generating, via a protocol generation module communicatively coupled to the at least one processor, a personalized intervention plan based on the determined system-level imbalances; and updating, via a feedback module communicatively coupled to the at least one processor, iteratively the personalized intervention plan based on subsequently acquired biological data to enable continuous recalibration of the physiological and emotional state of the user.

Other features and aspects of the disclosure will be apparent from the following description, list of elements, and the accompanying drawings.

### BRIEF DESCRIPTION OF THE DRAWINGS

Various embodiments of the present disclosure can be more readily understood and appreciated from the following descriptions of various embodiments of the present disclosure when read in conjunction with the accompanying drawings, in which:

FIG. 1 illustrates a network diagram of a biometric analysis system for tracing any conditions in a human body through changes in wave characteristics using an artificial intelligence (AI) technique, according to an embodiment of the present disclosure;

FIG. 2 illustrates a block diagram of the server in communication with the device and the user interface, according to an embodiment of the present disclosure;

FIG. 3 illustrates a flowchart showing a method for the biometric analysis system, according to an embodiment of the present disclosure;

FIGS. 4-11 illustrate the user interface with one or more options, according to an embodiment of the present disclosure;

FIGS. 12-21 illustrate card-index options and research analysis option for the user, according to an embodiment of the present disclosure;

FIGS. 22-35 illustrate etalons options, entropy analysis, and NLS analysis option, according to an embodiment of the present disclosure;

FIGS. 36-45 illustrate Meta therapy options, Vegeto test options, Reprinter options, and Backup options, according to an embodiment of the present disclosure; and

FIG. 46 illustrates a flowchart showing a computer-implemented method for biometric analysis, according to an embodiment of the present disclosure.

### DETAILED DESCRIPTION

Reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

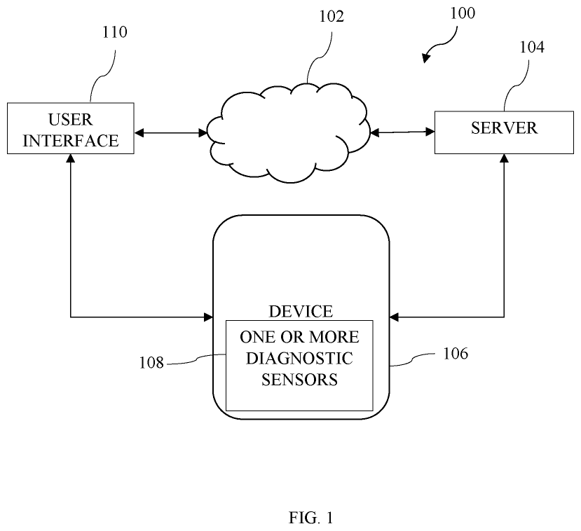

FIG. 1 illustrates a network diagram of a biometric analysis system 100 for tracing any conditions in a human body through changes in wave characteristics using an artificial intelligence (AI) technique, according to an embodiment of the present disclosure. The biometric analysis system 100 may comprise a network 102 communicatively coupled to a server 104, a device 106 having one or more diagnostic sensors 108 and a user interface 110.

In some embodiments, the network 102 may be a communication network such as internet or a cloud network, that may be configured to allow computing devices and processing systems to communicate with each other through wired network, wireless network, or a combination of both. In some embodiments, the network 102 may refer to as a distributed infrastructure that is configured to exchange of data, information, and resources among interconnected computing devices and systems. The network 102 may be designed to facilitate communication and collaboration across various locations, devices, and platforms. Those skilled in the art will recognize that wired devices may include, but are not limited to, wired networks such as Wide Area Networks (WANs) or Local Area Networks (LANs), while wireless devices may include wireless communications established via Radio Frequency (RF) signals or infrared signals. Various devices in the system 100 may connect to the network 102 in accordance with various wired and wireless communication protocols such as Transmission Control Protocol and Internet Protocol (TCP/IP), User Datagram Protocol (UDP), and 2G, 3G, or 4G communication protocols.

Further, the system 100 may comprise the server 104. In some embodiments, the server 104 may be a computer or software module that is configured to provide centralized resources, data, or services to the device 106 operated by the user. The server 104 may be configured to handle and manage one or more computational tasks and data processing within the system 100. In some embodiments, the server 104 may include storage systems, such as hard drives or storage arrays, to store and manage large volumes of data and information accessible to network users. In some embodiments, the server 104 may further provide centralized control and management capabilities, allowing network administrators to configure, monitor, and maintain network resources, security settings, and user access permissions from a single location.

In some embodiments, the server 104 may be configured for tracing any conditions in the human body through changes in the wave characteristics of tissue, individual cells, chromosomes or even separate ferments and hormones. In some embodiments, the server 104 may be configured to receive a plurality of test results of a user upon conducting one or more test of the user. In some embodiments, the plurality of tests comprises at least signal-based physiological analysis system, Biodecodification, Bloodwork, Hormone panel, and Dual-Energy X-ray Absorptiometry (DEXA) scan. Further, the server 104 may be configured to aggregate and analyse the plurality of test results to generate a personalized action plan for the user. In some embodiments, the personalized action plan is based on prevention, health optimization, longevity and anti-aging strategies, and addressing underlying dysfunctions.

Further, the biometric analysis system 100 comprises the device 106 having one or more diagnostic sensors 108. In some embodiments, the one or more diagnostic sensors 108 i.e. the diagnosis sensor may be integrated into any devices such as headphones etc., for transmitting electromagnetic waves to recognize specific frequencies (Vortex magnetic field) to detect functional status of the body, for the conducted plurality of tests i.e. signal-based physiological analysis system, Biodecodification, Bloodwork, Hormone panel, and DEXA scan.

The transmitted electromagnetic waves enable production of a preset bioelectrical activity of brain neurons (vibrational frequencies), to selectively amplify signals, and then isolate and decode the information they contain. In some embodiments, the server 104 is further configured to gather information about the specific temporary conditions and somatic imprints of organs and tissues on a non-contact basis by means of the one or more diagnostic sensors, and convert them to a digital sequence and transmitted to the server 104 to quantify the patient's quantified emotional load profile and other inferred physiological conditions. The one or more diagnostic sensors 108 are configured to scan specific emotions stored in body of the user, how those emotions impact cellular and organ-level function and the root causes of physiological and emotional imbalances.

In one embodiment, the Biodecodification or a biodecodification process is disclosed for assessing and addressing emotional and energetic imbalances potentially correlated with physical health conditions. The process involves the use of a Biodecodification Scanner system configured to detect and analyze vibrational frequency data in specific anatomical regions of a subject's body, with particular emphasis on organ systems. The Biodecodification Scanner captures frequency-based measurements associated with various regions of the subject's body. These measurements are then algorithmically compared to a reference dataset comprising baseline frequency values, including empirically derived data on emotional frequency spectrums. This comparison allows for the identification of anomalies or deviations indicative of potential emotional or energetic disruptions.

Subsequently, the collected frequency data is transmitted to the at least one processor 200 that aggregated and categorized into a set of key insights. These insights are used to generate a preliminary diagnostic profile of the subject. A structured interview or questionnaire is then administered to the subject or the user to further contextualize the data and guide interpretation. This stage enables identification of principal emotional and energetic pain points, and their spatial correlation to specific physical or physiological locations within the body. These correlations may, in certain instances, coincide with pre-existing or developing physiological health conditions. Following the analysis, the subject is optionally referred to one or more specialized service providers. These providers are selected based on their expertise in modalities capable of resolving the detected emotional conflicts or imbalances, such as counseling, energy therapy, or other holistic or integrative health services. In certain embodiments, a client-specific report is generated, detailing the findings of the vibrational frequency analysis, the emotional and physical correlations identified, and the recommended pathways for resolution.

In some embodiments, the server 104 are further configured that take bearings of this radiation (i.e. signals hardly detectable against the statistical fluctuations) just where it originates in order to then decode and correlate the somatic imprints to emotional state of the patient to quantify the user (i.e. patient's) quantified emotional load profile and other inferred physiological conditions and display it on the user interface 110 where a virtual model of an organ is produced in specific colors/patterns. The specific colors are produced using rules of quantum chromo kinetics where the hardware and software modules represent entropy values of any system as spectrum colors, the tints will change from light yellow (minimum entropy values), through orange to red and purple, to nearly black (maximum entropy values). The server 104 are configured to initiate accurate theoretical calculations that enables the singling out a number of stationary states corresponding to certain entropy potential which then selectively interact with the spectrum of electromagnetic radiation.

In some embodiments, the server 104 is further configured to give physicians a three-dimensional projection of the internal organs. The coloured marks placed upon the picture make it easier for the doctor to determine regions associated with functional deviations or system-level imbalances. The server 104 when after reading frequency characteristics of the researched biological object, the server 104 compares the degree of their spectral similarity with healthy, and pathologically affected tissue, or infection agents, to obtain the closest pathological processor tendency. In some embodiments, the biometric analysis system 100 provides a unique opportunity of recording the frequency fluctuations of any preparation and adding them to the many thousands already held in the database. In some embodiments, the biometric analysis system 100 using the server 104, is configured to search for a remedy that has the closest spectral characteristic of the pathological process and display personalized action plan having most efficient remedy, prevention, health optimization, Longevity and anti-aging strategies, and addressing underlying dysfunctions which are displayed to the patient. The health optimization refers to bringing body back to its optimal level of performance, ideally to homeostasis-self healing process.

Further, the biometric analysis system 100 comprises a user interface 110. In some embodiments, the user interface 110 provides a user-friendly platform for the user. The user interface 110 may be web-based, accessed through a browser, or through a dedicated software application installed on desktop computers, laptops, tablets, or smartphone. The user interface 110 may be equipped by the user or other service professionals. In some embodiments, the user interface 110 may include personal computers such as desktop computers, laptop computers, tablets, smartphones, or mobile devices. The user interface 110 comprises a plurality of icons such as main menu having icons as “reception of patients”, “about”, “settings”, and “exit”. Other icons include, “Report”, “Search”, “Patient's card” containing his/her personal data, such as: surname, name, patronymic, age, sex, blood group, address, phone number, “Research” allows to carry out computer nonlinear analysis in the mode of a programmed and (or) individual selection of the organs intended for researches.

Further, in “Researches” mode user can measure the frankness of the destructive process at reference points standardly placed on the picture. The main feature of the software is “scan” function, which allows automatic identification of regions associated with functional deviations or patterns of physiological imbalance within the user. The icon “settings” displays the setup form where the user or operator can turn on and off sound, change font size, make the graphs background in white and black, etc. A button “Select doctor” allows selecting from the list the doctor, who will carry out the researches. A button “Color in card-index” allows the user to highlight organs with different functional stages from already made researches. Green line shows there is no pronounced functional change in evidence. Red line shows minor functional changes. Brown line shows pronounced functional and/or organic changes. More features include entropy analysis, NLS analysis, meta therapy, vegeto test, or reprinter.

In some embodiments, the one or more diagnostic sensors 108 integrated into the device 106 is configured to be positioned in proximity to the user. In some embodiments, the one or more diagnostic sensors 108 is configured to transmit and receive electromagnetic excitation signals to acquire biological data from the user, the biological data comprising at least bioelectrical activity, electromagnetic wave characteristics, biofrequency patterns, and physiological parameters associated with physiological activity of one or more tissues, one or more organs, and cellular activity. In some embodiments, the one or more diagnostic sensors 108 integrated into the device 106 are configured to be positioned in proximity to the user, including in a non-contact or near-field arrangement relative to a body surface of the user. The device 106 may be implemented as a wearable device, a handheld device, or a user-adjacent device, including but not limited to a head-mounted device, headphones, a chest-mounted device, or any apparatus capable of maintaining a consistent spatial relationship with the user for signal acquisition.

The positioning of the one or more diagnostic sensors 108 enables detection of electromagnetic and bioelectrical emissions associated with underlying biological tissues without requiring invasive measurement. In some embodiments, the one or more diagnostic sensors 108 are configured to transmit and receive electromagnetic excitation signals to acquire biological data from the user. The transmitted electromagnetic excitation signals may comprise low-energy excitation signals, scanning signals, or probing signals configured to interact with biological tissues, while the received electromagnetic excitation signals may comprise reflected, modulated, or emitted signals corresponding to intrinsic bioelectrical activity and tissue-specific electromagnetic responses. The one or more diagnostic sensors 108 may include, without limitation, electromagnetic field sensors, biofield sensors, impedance sensors, galvanic response sensors, frequency resonance sensors, or combinations thereof, configured to capture multi-dimensional signal characteristics.

In some embodiments, the acquired biological data comprises bioelectrical activity indicative of cellular membrane potentials and electrochemical signaling, electromagnetic wave characteristics corresponding to tissue-specific resonance and propagation properties, biofrequency patterns representing oscillatory behavior across biological systems, and physiological parameters associated with physiological activity of one or more tissues, one or more organs, and cellular activity. The physiological parameters may include indicators of autonomic nervous system activity, metabolic state, inflammatory response, and tissue-level electrical deviations. In some embodiments, the acquired biological data further reflects spatial and regional variations across the body, enabling identification of localized signal deviations corresponding to specific anatomical regions or organ systems.

Such variations may arise from differences in tissue composition, fascial structure, or localized physiological conditions, including chronic stress-induced changes in connective tissue. In particular, fascial tissue exhibiting piezoelectric properties may generate measurable electrical responses under mechanical or physiological stress, thereby contributing to the detected biofrequency patterns. In some embodiments, the one or more diagnostic sensors 108 are configured to capture dynamic temporal variations in the biological data, including baseline measurements and responsive changes under varying physiological or environmental conditions. The biological data may therefore represent both steady-state and transient characteristics of the user's physiological and bioelectrical state, enabling downstream processing of signal characteristics representative of functional states of the user.

In some embodiments, the one or more diagnostic sensors 108 comprise electromagnetic sensors configured to transmit and receive the electromagnetic excitation signals to acquire the biofrequency patterns corresponding to resonance characteristics of the one or more tissues or the one or more organs of the user. In some embodiments, the one or more diagnostic sensors comprise at least one of an electromagnetic transceiver, a biofrequency sensor, a resonance-based sensor, or a bioimpedance sensor.

Further, the server 104 is configured to receive the biological data and extract signal characteristics comprising frequency-domain features and waveform-based features representative of functional states of the user. The server 104 is configured to receive the biological data from the device 106 via a communication network and to process the received biological data to extract signal characteristics comprising frequency-domain features and waveform-based features representative of functional states of the user. In some embodiments, the biological data is received as raw or pre-processed electromagnetic signal data corresponding to bioelectrical activity, biofrequency patterns, and physiological responses detected by the one or more diagnostic sensors 108. The extracted signal characteristics are used to infer functional states of the user based on patterns in the biological data.

In some embodiments, the server 104 is configured to perform signal preprocessing operations including filtering, noise reduction, normalization, and signal segmentation to isolate relevant signal components from background interference and environmental noise. Such preprocessing may include removal of statistical fluctuations, correction of signal distortions, and enhancement of signal-to-noise ratio to enable accurate downstream analysis of low-amplitude biofrequency signals associated with biological tissues. In some embodiments, the extraction of frequency-domain features comprises transforming the biological data into a frequency representation using one or more signal transformation techniques, including but not limited to Fourier transform, wavelet transform, or spectral decomposition methods. The frequency-domain features may include dominant frequency components, resonance peaks, spectral density distributions, harmonic signatures, and frequency deviations indicative of physiological conditions or tissue-level dysregulation.

In some embodiments, the extraction of waveform-based features comprises analyzing time-domain characteristics of the biological data, including amplitude variations, phase relationships, temporal patterns, signal morphology, and transient responses. These waveform-based features may capture dynamic physiological responses, including variations associated with autonomic nervous system activity, metabolic fluctuations, or localized tissue responses. In some embodiments, the extracted signal characteristics represent functional states of the user by encoding information related to physiological regulation, including autonomic balance, inflammatory activity, metabolic efficiency, and recovery capacity. The functional states may further reflect spatially localized variations in signal characteristics corresponding to specific anatomical regions, organ systems, or fascial structures, thereby enabling identification of localized deviations associated with somatic encoding of physiological and emotional conditions. The extraction of signal characteristics forms part of a signal processing pipeline configured to transform raw biological data into structured representations for downstream analysis.

In some embodiments, the server 104 is configured to generate a structured representation of the extracted signal characteristics, enabling subsequent processing by downstream modules including decoding, correlation, and analysis components. The extracted features therefore form a foundational data layer for interpreting the biological data in terms of physiological conditions and system-level imbalances. In some embodiments, the server 104 is configured to convert the extracted signal characteristics into digital representations representing inferred physiological conditions and somatic imprints representing inferred functional states of the one or more organs or biological systems based on the extracted signal characteristics. The digital representations may comprise structured data formats, multi-dimensional feature vectors, parameterized models, or encoded state variables that quantitatively represent relationships between the extracted signal characteristics and underlying biological functions.

In some embodiments, the server 104 is configured to interpret the frequency-domain features and waveform-based features and map the extracted signal characteristics to corresponding physiological conditions. Such interpretation may include identification of deviations from baseline frequency patterns, detection of resonance characteristics associated with specific tissues or organ systems, and recognition of signal signatures indicative of physiological states including inflammation, metabolic imbalance, autonomic nervous system activity, or recovery capacity. In some embodiments, the server 104 is further configured to apply one or more computational models, including trained artificial intelligence models, to establish relationships between the extracted signal characteristics and the physiological conditions.

The trained models may be configured to learn associations between biofrequency patterns and known biological states, thereby enabling inference of physiological conditions based on observed signal characteristics. The conversion process may therefore include pattern recognition, classification, clustering, or regression-based interpretation of the extracted features. In some embodiments, the digital representations further comprise somatic imprints representing functional states of the one or more organs or biological systems. The somatic imprints may correspond to localized or system-level patterns of physiological deviation encoded within biological tissues, including patterns associated with chronic stress, autonomic dysregulation, or tissue-level electrical variation. Such somatic imprints may be derived from spatially or regionally resolved signal characteristics, enabling association of specific signal patterns with particular anatomical regions, organ systems, or fascial structures.

In some embodiments, the somatic imprints capture bioelectrical and biofrequency signatures indicative of persistent or historical physiological conditions, including those influenced by sustained emotional or neurological states. The somatic imprints may therefore represent a structured encoding of the user's physiological and regulatory state, forming an intermediate representation for subsequent analysis performed by the server 104. In some embodiments, the server 104 is further configured to store, update, and refine the digital representations over time based on subsequently received biological data, thereby enabling adaptive and progressively refined characterization of the physiological conditions and somatic imprints of the user.

In some embodiments, the server 104 is further configured to correlate the somatic imprints with emotional, neurological, and behavioral states of the user by applying one or more artificial intelligence (AI) models configured to infer relationships between physiological representations and emotional states, and generate a quantified emotional load profile based on the determined relationships. The correlation may include associating the somatic imprints, which represent localized or system-level physiological deviations, with corresponding emotional patterns and neurological states reflected in the biological data.

In some embodiments, the server 104 is configured to apply the one or more AI models to identify patterns linking physiological conditions of the one or more organs or biological systems with emotional and behavioral states of the user. The AI models may be trained on reference datasets comprising known associations between biofrequency patterns, autonomic nervous system responses, and emotional conditions, thereby enabling inference of emotional states from the somatic imprints. The inferred emotional states may include, without limitation, stress, anxiety, depressive states, heightened arousal, reduced regulatory capacity, or other emotional or neurological conditions associated with physiological dysregulation.

In some embodiments, the correlation performed by the server 104 further incorporates relationships between organ systems and emotional states, wherein specific physiological conditions associated with particular organs or biological systems are linked to corresponding emotional patterns. Such relationships may be derived from empirical datasets, clinical mappings, or integrative physiological models, thereby enabling interpretation of the somatic imprints in terms of emotional and neurological influences on the user's physiological state.

In some embodiments, the server 104 is configured to generate the quantified emotional load profile as a structured representation of the emotional and neurological burden associated with the user. The quantified emotional load profile may comprise numerical values, weighted scores, multi-dimensional vectors, or probabilistic measures representing the intensity, distribution, and relative contribution of different emotional states across the one or more organs or biological systems. The quantified emotional load profile may further reflect temporal and spatial variations in emotional load, including localized concentrations of emotional burden corresponding to specific anatomical regions or organ systems.

In some embodiments, the quantified emotional load profile provides an integrated representation of the interaction between physiological conditions and emotional states, enabling identification of systemic patterns of dysregulation arising from sustained emotional or neurological influences. The quantified emotional load profile may therefore serve as an input for subsequent evaluation of system-level imbalances and generation of personalized intervention plans. In some embodiments, the server 104 is further configured to update the quantified emotional load profile over time based on subsequently acquired biological data, thereby enabling dynamic tracking of changes in emotional and physiological states and facilitating continuous assessment of the user's regulatory condition.

In some embodiments, the server 104 is further configured to map the physiological conditions and the quantified emotional load profile to corresponding reference parameters within the reference data indicative of healthy and pathological states, by applying the one or more AI models configured to learn relationships between the physiological conditions, emotional load factors, and the reference parameters. The mapping may include selecting, aligning, and associating specific elements of the physiological conditions and the quantified emotional load profile with relevant parameters within the reference data, such that the comparison is performed using contextually appropriate and dynamically determined reference criteria.

In some embodiments, the reference data comprises datasets representing baseline and pathological conditions associated with physiological activity of one or more tissues, one or more organs, and biological systems, including reference biofrequency patterns, physiological ranges, and regulatory states. The reference parameters may include frequency signatures, spectral characteristics, physiological thresholds, and multi-dimensional indicators of system health corresponding to both normal and abnormal conditions. In some embodiments, the server 104 is configured to apply the one or more AI models to identify relationships between the physiological conditions, the quantified emotional load profile, and the reference parameters, thereby enabling adaptive determination of which reference parameters are relevant for evaluating the current state of the user. The mapping may therefore include weighting, prioritization, or selection of reference parameters based on the combined influence of physiological and emotional factors represented in the quantified emotional load profile.

In some embodiments, the mapping performed by the server 104 enables integration of multi-domain data, including physiological, emotional, and neurological information, into a unified reference framework. This allows the server 104 to account for interactions between physiological conditions and emotional states when selecting corresponding reference parameters, thereby improving the accuracy and contextual relevance of subsequent evaluation of system-level imbalances. In some embodiments, the mapping further includes generating mapped representations of the physiological conditions and the quantified emotional load profile, wherein the mapped representations correspond to selected reference parameters within the reference data. The mapped representations may comprise structured data linking specific physiological conditions and emotional load factors to corresponding healthy or pathological reference ranges, thereby enabling targeted and efficient comparison.

In some embodiments, the server 104 is further configured to update the mapping dynamically based on newly acquired biological data and evolving physiological and emotional states of the user, thereby enabling continuous adaptation of the reference parameter selection and improving the robustness of system-level analysis over time. Further, the server 104 is configured to evaluate the physiological conditions and the quantified emotional load profile mapped to the corresponding reference parameters against reference data indicative of healthy and pathological states to determine system-level imbalances comprising at least one of inflammation, stress response, metabolic efficiency, and recovery capacity.

The evaluation may include comparing the mapped physiological conditions and emotional load factors with corresponding reference parameters to identify deviations from baseline or optimal regulatory states associated with the one or more tissues, one or more organs, or biological systems. In some embodiments, the server 104 is configured to determine system-level imbalances by identifying variations in frequency characteristics, spectral distributions, and physiological indicators relative to the reference data. Such variations may indicate dysregulation in biological systems, including elevated inflammatory activity, altered autonomic nervous system balance, impaired metabolic function, or reduced capacity for physiological recovery.

In some embodiments, the determination of inflammation may be based on detection of signal patterns indicative of tissue-level stress responses, immune activation, or deviations in bioelectrical characteristics associated with inflammatory processes. The determination of stress response may include analysis of signal characteristics associated with autonomic nervous system activity, including sympathetic activation or parasympathetic suppression reflected in the biological data. In some embodiments, the determination of metabolic efficiency may include evaluation of signal characteristics associated with cellular energy utilization, electrochemical activity, and tissue-level metabolic function, as inferred from the extracted signal characteristics and the corresponding digital representations. The determination of recovery capacity may include assessment of the ability of the user's physiological systems to return to baseline states following perturbation, as indicated by temporal variations and regulatory patterns in the biological data.

In some embodiments, the evaluation performed by the server 104 integrates both physiological conditions and the quantified emotional load profile, thereby enabling identification of system-level imbalances arising from interactions between physiological and emotional factors. The evaluation may therefore account for compounded effects of physiological dysregulation and emotional burden on the overall functional state of the user. In some embodiments, the server 104 is further configured to generate an evaluation output comprising one or more indicators, scores, or classifications representing the identified system-level imbalances. The evaluation output may be used for subsequent generation of personalized intervention plans and for monitoring changes in the user's physiological and emotional state over time.

Further, the server 104 is configured to generate a personalized intervention plan based on the determined system-level imbalances. The personalized intervention plan may comprise a structured set of intervention protocols tailored to the physiological conditions and the quantified emotional load profile of the user, wherein the intervention protocols are configured to address the identified system-level imbalances in a targeted and adaptive manner. In some embodiments, the personalized intervention plan is generated by selecting and organizing one or more intervention modalities based on the type, severity, and distribution of the system-level imbalances. The intervention modalities may include, without limitation, biochemical interventions, nutritional supplementation, peptide-based therapies, neuromodulation techniques, behavioral modifications, activity and lifestyle adjustments, and mind-body or somatic therapies configured to regulate physiological and emotional states of the user.

In some embodiments, the personalized intervention plan is configured to address both physiological and emotional components of the system-level imbalances by integrating multi-domain interventions targeting biological regulation, neurological function, and emotional patterning. The intervention plan may therefore include protocols directed toward reducing inflammation, improving metabolic efficiency, enhancing recovery capacity, and modulating autonomic nervous system activity. In some embodiments, the personalized intervention plan is structured in a sequential or phased manner, wherein the interventions are arranged according to a progression of system regulation and optimization. The sequential structure may include an initial phase configured to reduce physiological interference and inflammatory load, a subsequent phase configured to provide targeted biochemical and nutritional support, a further phase configured to modify neural, emotional, and behavioral response patterns, and a subsequent phase configured to enhance overall physiological and cognitive performance.

In some embodiments, the sequencing of the personalized intervention plan is determined based on the relative severity and priority of the system-level imbalances, such that interventions are applied in an order corresponding to the readiness of the user's physiological systems for regulation and recovery. The personalized intervention plan may therefore be dynamically adjusted to align with changes in the user's physiological and emotional state over time. In some embodiments, the personalized intervention plan further comprises one or more actionable recommendations, schedules, or protocols configured for implementation by the user, and may be communicated to the user via a user interface or external computing device associated with the system.

Further, the server 104 is configured to iteratively update the personalized intervention plan based on subsequently acquired biological data to enable continuous recalibration of the physiological and emotional state of the user. The subsequently acquired biological data may be obtained through repeated or periodic measurements performed by the one or more diagnostic sensors 108, thereby capturing temporal changes in the user's physiological conditions and quantified emotional load profile. In some embodiments, the server 104 is configured to process the subsequently acquired biological data in a manner consistent with previously described processing steps, including extraction of signal characteristics, conversion into digital representations, correlation with emotional and neurological states, mapping to reference parameters, and evaluation of system-level imbalances. The updated system-level imbalances are then used to modify, refine, or reconfigure the personalized intervention plan.

In some embodiments, the iterative updating includes adjusting one or more aspects of the personalized intervention plan, including modification of intervention modalities, adjustment of dosage or intensity of interventions, reordering of intervention sequences, or introduction of additional intervention protocols based on the updated physiological conditions and quantified emotional load profile. The updating may therefore reflect improvements, regressions, or newly identified patterns in the user's physiological and emotional state. In some embodiments, the server 104 is configured to implement a closed-loop feedback mechanism, wherein outputs of the personalized intervention plan influence subsequent biological data, and the subsequently acquired biological data is used to further refine the intervention plan. This closed-loop system enables adaptive optimization of the intervention plan based on real-time or near real-time feedback from the user's biological system.

In some embodiments, the server 104 is further configured to track longitudinal changes in the physiological conditions and the quantified emotional load profile, thereby enabling monitoring of trends associated with recovery, regulation, or progression of system-level imbalances. The iterative updates may therefore support progressive improvement in system performance, including reduction in inflammation, stabilization of emotional states, enhancement of metabolic efficiency, and improvement in recovery capacity. In some embodiments, the continuous recalibration performed by the server 104 enables the personalized intervention plan to remain aligned with the evolving physiological and emotional state of the user over extended periods of time, thereby facilitating sustained regulation and long-term optimization of system-level function.

Further, the server 104 is configured to present the inferred physiological conditions, the quantified emotional load profile, and the personalized intervention plan. In some embodiments, the server 104 is configured to generate output data corresponding to the physiological conditions, the quantified emotional load profile, and the personalized intervention plan, and to transmit the output data to the user interface 110 executed on at least one of the device 106 or an external computing device communicatively coupled to the server 104, including but not limited to a mobile device, tablet, or computer system.

In some embodiments, the presented physiological conditions comprise visual, numerical, or graphical representations of the functional states of the one or more tissues, one or more organs, or biological systems, including indicators of system-level imbalances such as inflammation, stress response, metabolic efficiency, and recovery capacity. The quantified emotional load profile may be presented as a structured visualization comprising scores, distributions, or graphical indicators representing the intensity and localization of emotional and neurological states across the one or more organs or biological systems. In some embodiments, the server 104 is further configured to present a three-dimensional representation of the one or more organs or biological systems of the user, wherein the three-dimensional representation includes one or more visual markers corresponding to locations of physiological conditions or system-level imbalances.

The visual markers may indicate localized regions of dysregulation, thereby enabling identification of anatomical regions associated with the somatic imprints. In some embodiments, the server 104 is configured to present the physiological conditions and the quantified emotional load profile using a color spectrum corresponding to entropy values associated with the physiological conditions or the quantified emotional load profile. The color spectrum may comprise a gradient ranging from lighter colors representing lower entropy values to darker colors representing higher entropy values, thereby enabling intuitive visualization of the relative severity and distribution of system-level imbalances.

In some embodiments, the personalized intervention plan is presented as a structured set of actionable recommendations, schedules, or protocols, including indications of sequencing, priority, and progression of intervention phases. The presentation may further include guidance for implementation of intervention modalities, progress tracking indicators, and updates reflecting iterative recalibration of the personalized intervention plan. In some embodiments, the server 104 is configured to update the presented information in real time or near real time based on newly acquired biological data, thereby enabling continuous monitoring and visualization of changes in the physiological conditions, the quantified emotional load profile, and the personalized intervention plan.

In some embodiments, the server 104 using the user interface 110 is further configured to present the physiological conditions and the quantified emotional load profile using a color spectrum corresponding to entropy values associated with the physiological conditions or the quantified emotional load profile. The color spectrum comprises a gradient ranging from lighter colors representing lower entropy values to darker colors representing higher entropy values. The entropy values may represent a measure of disorder, variability, or deviation of the physiological conditions and the quantified emotional load profile from corresponding reference parameters indicative of healthy or regulated states.

In some embodiments, the server 104 is configured to compute the entropy values based on variations in the mapped physiological conditions and the quantified emotional load profile relative to baseline or reference data. The entropy values may be derived from statistical measures, signal variability, spectral dispersion, or multi-dimensional deviations associated with the biological data, thereby providing a quantitative indicator of system-level imbalance. In some embodiments, the color spectrum comprises a gradient ranging from lighter colors representing lower entropy values to darker colors representing higher entropy values. The lighter colors may correspond to states of higher physiological coherence, stability, or regulation, while the darker colors may correspond to states of increased physiological disruption, imbalance, or elevated emotional load.

The gradient may include intermediate color transitions to represent varying degrees of deviation across the physiological conditions and the quantified emotional load profile. In some embodiments, the server 104 is configured to apply the color spectrum to one or more visual representations presented via the user interface 110, including graphical displays, charts, or anatomical representations of the one or more organs or biological systems. The color mapping may be applied to localized regions corresponding to specific tissues or organ systems, thereby enabling spatial visualization of entropy values and facilitating identification of areas associated with higher system-level imbalances.

In some embodiments, the color-based representation enables intuitive interpretation of the physiological conditions and the quantified emotional load profile by providing a visual indication of severity, distribution, and progression of system-level imbalances. The color spectrum may further be dynamically updated based on subsequently acquired biological data, thereby reflecting changes in entropy values over time and supporting monitoring of improvement or deterioration in the user's physiological and emotional state. Further, the server 104 using the user interface 110 is further configured to present a three-dimensional representation of one or more organs or biological systems of the user, and to display one or more visual markers on the three-dimensional representation corresponding to locations of the physiological conditions or system-level imbalances.

In some embodiments, the server 104 is configured to generate the three-dimensional representation based on anatomical models, digital organ templates, or user-specific data, thereby providing a spatially accurate visualization of the one or more organs or biological systems. In some embodiments, the server 104 is configured to map the physiological conditions and the system-level imbalances to corresponding anatomical locations within the three-dimensional representation based on the somatic imprints and spatial characteristics of the biological data. The mapping may include associating localized signal characteristics, regional biofrequency deviations, or organ-specific physiological conditions with corresponding regions of the three-dimensional model.

In some embodiments, the one or more visual markers comprise graphical indicators, color-coded regions, overlays, or symbols displayed on the three-dimensional representation to indicate the presence, location, and relative severity of the physiological conditions or system-level imbalances. The visual markers may be dynamically generated based on the evaluation output and may vary in size, shape, intensity, or color to represent different levels of imbalance. In some embodiments, the visual markers are presented in combination with the color spectrum corresponding to entropy values, such that the color of each visual marker or region reflects the associated entropy value of the physiological conditions or the quantified emotional load profile. This enables simultaneous visualization of both spatial location and severity of system-level imbalances across the one or more organs or biological systems.

In some embodiments, the server 104 is configured to enable interaction with the three-dimensional representation via the user interface 110, including rotation, zooming, selection of specific organs or regions, and display of additional information associated with selected visual markers. The additional information may include corresponding physiological conditions, quantified emotional load values, and relevant portions of the personalized intervention plan associated with the selected region. In some embodiments, the three-dimensional representation and the associated visual markers are updated dynamically based on subsequently acquired biological data, thereby enabling continuous visualization of changes in the physiological conditions and system-level imbalances over time. The outputs generated by the biometric analysis system 100 represent inferred physiological conditions and are not intended to constitute clinical diagnoses, but rather provide indicative information for system-level interpretation and optimization.

In some embodiments, the biometric analysis system 100 is configured as an integrated platform that combines multiple sensing and analytical modalities into a unified system. The modalities may include sensor-based measurements, frequency-based signal analysis, and other non-invasive data acquisition techniques, wherein outputs from the multiple modalities are processed collectively to generate a coherent representation of the user's physiological and emotional state. In some embodiments, the at least one processor 200 implements a processing layer configured to aggregate, normalize, and analyze data from the multiple modalities, thereby enabling unified interpretation of heterogeneous biological data. In some embodiments, the biometric analysis system 100 is configured to translate interpreted signal data into actionable outputs, including personalized recommendations comprising at least one of nutritional supplementation, botanical support, bio-optimization strategies, and preventive intervention pathways.

It will be apparent to one skilled in the art that above-mentioned components of the biometric analysis system 100 have been provided only for illustration purposes, without departing from the scope of the disclosure.

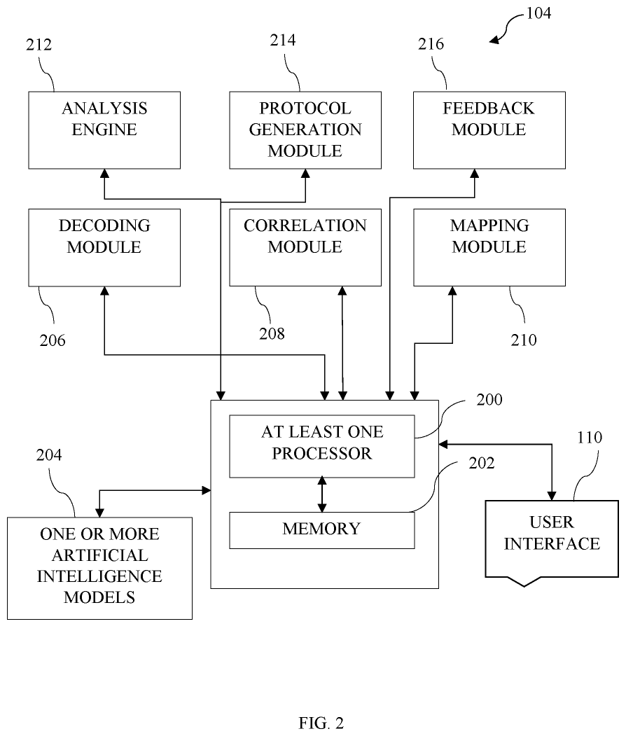

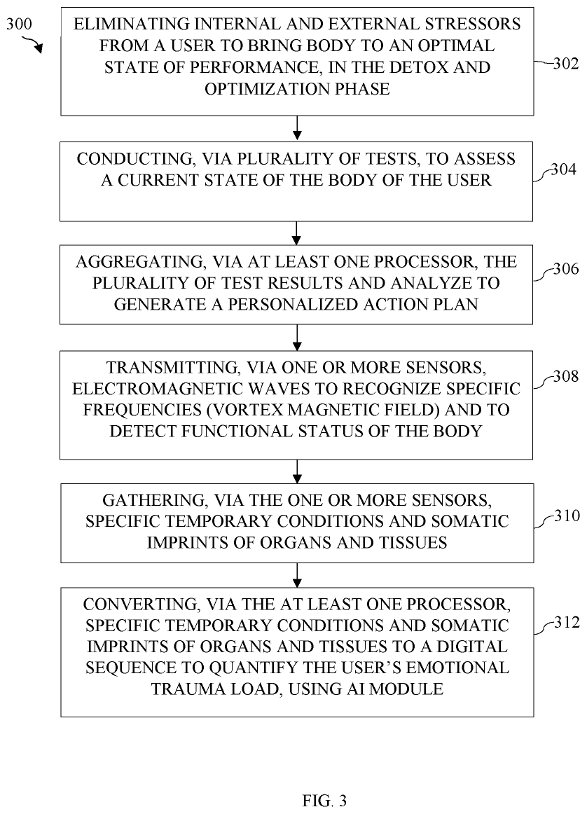

FIG. 2 illustrates a block diagram of the server 104 in communication with the device 106 and the user interface 110, according to an embodiment of the present disclosure. FIG. 3 illustrates a flowchart showing a method 300 for the biometric analysis system 100, according to an embodiment of the present disclosure.

In some embodiments, the server 104 comprises at least one processor 200, a memory 202 and one or more artificial intelligence (AI) models 204. Further, the FIG. 2 shows the communication between the server 104, the device 106 and the user interface 110. In some embodiments, the biometric analysis system 100 is configured for removal of obstructions, physiological and emotions, that prevent the body and mind from operating at highest potential of the user. In some embodiments, the biometric analysis system 100 is configured to follow a plurality of phases for the removal of obstructions, physiological and emotions that prevent the body and mind from operating at highest potential of the user. It may be noted that the server 104 may be in form of an apparatus (not shown) and thereby the server 104 and the apparatus may be interchangeably used. In some embodiments, the first of the plurality of phases include detox and optimization phase comprising diagnostic and data collection phase and assessment and protocol generation phase and the second of the plurality of phases include emotional detox and biodecodification phase.

Further, the server 104 comprises a decoding module 206, a corelation module 208, a mapping module 210, an analysis engine 212, a protocol generation module 214, a feedback module 216. In some embodiments, as described earlier, the one or more diagnostic sensors 108 integrated into the device 106 configured to be positioned in proximity to the user. In some embodiments, the one or more diagnostic sensors 108 is configured to transmit and receive electromagnetic excitation signals to acquire biological data from the user, the biological data comprising at least bioelectrical activity, electromagnetic wave characteristics, biofrequency patterns, and physiological parameters associated with physiological activity of one or more tissues, one or more organs, and cellular activity. In some embodiments, the device 106 comprises a standalone system configured to integrate multiple sensing and signal delivery components within a unified physical structure, wherein the device 106 enables interaction of the user with multiple embedded technologies through a single interface.

In some embodiments, the one or more diagnostic sensors 108 are configured to transmit and receive electromagnetic excitation signals to acquire biological data from the user, wherein the acquired biological data is communicated to the at least one processor 200 for further processing by one or more of the decoding module 206, the correlation module 208, the mapping module 210, the analysis engine 212, the protocol generation module 214, and the feedback module 216. The transmission of electromagnetic excitation signals may include emission of low-energy probing or excitation signals toward the user, and the reception may include detection of reflected, modulated, or emitted electromagnetic responses corresponding to intrinsic bioelectrical activity of biological tissues.

In some embodiments, the one or more diagnostic sensors 108 are configured to capture biological data comprising bioelectrical activity indicative of cellular membrane potentials and electrochemical signaling, electromagnetic wave characteristics corresponding to tissue-specific resonance and propagation properties, and biofrequency patterns representing oscillatory behavior across one or more biological systems. The biological data may further include physiological parameters associated with physiological activity of one or more tissues, one or more organs, and cellular activity, including indicators of autonomic nervous system activity, metabolic state, inflammatory response, and tissue-level electrical variation.

In some embodiments, the one or more diagnostic sensors 108 are configured to acquire multi-dimensional signal data across a range of frequencies and amplitudes, enabling detection of both steady-state and dynamic variations in the biological signals. The acquired biological data may reflect spatial variations across different anatomical regions of the user, including localized signal deviations associated with specific organs or fascial structures, thereby providing input for generation of somatic imprints by the decoding module 206. In some embodiments, the biometric analysis system 100 utilizes multiple analytical modalities including frequency-based signal analysis, non-linear signal interpretation, and bioelectrical signal acquisition techniques, wherein each modality contributes complementary information for system-level analysis.

In some embodiments, the one or more diagnostic sensors 108 are configured to capture low-amplitude electromagnetic emissions and biofrequency signals that may be embedded within background noise, wherein the acquired signals are subsequently processed by the at least one processor 200 to enhance signal quality, isolate relevant signal components, and prepare the biological data for downstream processing by the decoding module 206 and subsequent modules. In some embodiments, the biological data acquired by the one or more diagnostic sensors 108 is continuously or periodically transmitted to the server 104, thereby enabling real-time or near real-time processing of the biological data by the at least one processor 200 and the associated modules for dynamic assessment of physiological conditions and emotional states of the user.

In some embodiments, the at least one processor 200 is communicatively coupled to the one or more diagnostic sensors 108 and the memory 202 storing instructions, where the at least one processor 200, upon executing the instructions, is configured to receive the biological data and extract signal characteristics comprising frequency-domain features and waveform-based features representative of functional states of the user. The biological data received by the at least one processor 200 may comprise raw or pre-processed electromagnetic excitation signals corresponding to bioelectrical activity, biofrequency patterns, and physiological responses detected by the one or more diagnostic sensors 108.

In some embodiments, the at least one processor 200 is configured to perform one or more signal preprocessing operations prior to feature extraction, including filtering, denoising, normalization, and segmentation of the biological data to isolate relevant signal components and reduce background interference. Such preprocessing may include removal of statistical fluctuations, compensation for signal distortions, and enhancement of signal-to-noise ratio to enable accurate analysis of low-amplitude biofrequency signals. In some embodiments, the extraction of frequency-domain features by the at least one processor 200 comprises transforming the biological data into a frequency representation using one or more signal transformation techniques, including but not limited to Fourier transform, wavelet transform, or spectral decomposition methods. The frequency-domain features may include dominant frequency components, resonance peaks, spectral density distributions, harmonic structures, and frequency deviations indicative of physiological conditions associated with physiological activity of one or more tissues or organs.

In some embodiments, the extraction of waveform-based features comprises analyzing time-domain characteristics of the biological data, including amplitude variations, phase relationships, temporal patterns, signal morphology, and transient responses. The waveform-based features may capture dynamic physiological behavior, including variations associated with autonomic nervous system activity, metabolic fluctuations, or localized tissue responses. In some embodiments, the extracted signal characteristics collectively represent functional states of the user by encoding information related to physiological regulation, including autonomic balance, inflammatory activity, metabolic efficiency, and recovery capacity. The extracted features may further reflect spatially localized variations corresponding to specific anatomical regions, organ systems, or fascial structures, thereby enabling downstream processing by the decoding module 206 for generation of physiological conditions and somatic imprints.

In some embodiments, the at least one processor 200 is further configured to organize the extracted signal characteristics into structured data representations suitable for subsequent processing by the decoding module 206, the correlation module 208, and the mapping module 210, thereby forming an intermediate representation layer for interpreting the biological data. In some embodiments, the at least one processor 200 is further configured to convert, via the decoding module 206 communicatively coupled to the at least one processor 200, the extracted signal characteristics into digital representations representing inferred physiological conditions and somatic imprints representing inferred functional states of the one or more organs or biological systems based on the extracted signal characteristics. The conversion may include transforming the frequency-domain features and waveform-based features into structured representations that encode relationships between the extracted signal characteristics and underlying biological processes. In some embodiments, the decoding process is performed using one or more artificial intelligence models configured to learn relationships between signal characteristics and physiological conditions, thereby enabling data-driven inference of functional states.

In some embodiments, the at least one processor 200 is configured to interpret the extracted signal characteristics to identify patterns, deviations, and resonance behavior associated with physiological activity of one or more tissues, one or more organs, or biological systems. The conversion may include mapping frequency signatures, spectral distributions, and waveform characteristics to corresponding physiological conditions, including indicators of inflammation, metabolic imbalance, autonomic nervous system activity, and recovery capacity. In some embodiments, the at least one processor 200 is further configured to apply one or more artificial intelligence models 204 to infer relationships between the extracted signal characteristics and the physiological conditions. The one or more artificial intelligence models 204 may be trained using reference datasets comprising known associations between biofrequency patterns and biological states, thereby enabling inference of physiological conditions based on the extracted signal characteristics. The conversion process may therefore include pattern recognition, classification, clustering, or regression-based interpretation of the extracted features.

In some embodiments, the digital representations further comprise somatic imprints representing functional states of the one or more organs or biological systems. The somatic imprints may correspond to localized or system-level patterns of physiological deviation encoded within biological tissues, including patterns associated with chronic stress, autonomic dysregulation, or tissue-level electrical variation. The somatic imprints may be derived from spatially or regionally resolved signal characteristics, enabling association of specific signal patterns with particular anatomical regions, organ systems, or fascial structures. In some embodiments, the digital representations generated by the at least one processor 200 provide an intermediate representation layer that captures both physiological conditions and somatic imprints, thereby enabling subsequent processing by the correlation module 208, the mapping module 210, and the analysis engine 212.

In some embodiments, the decoding module 206 is configured to process the extracted signal characteristics to generate the digital representations corresponding to physiological conditions and somatic imprints. The decoding module 206 may comprise one or more software components, hardware components, or a combination thereof, executed by the at least one processor 200. In some embodiments, the decoding module 206 is configured to perform feature interpretation, pattern recognition, and signal-to-physiology mapping operations, including identification of frequency signatures, resonance characteristics, and waveform patterns associated with specific tissues, organs, or biological systems. The decoding module 206 may further be configured to utilize the one or more artificial intelligence models 204 to establish relationships between the extracted signal characteristics and physiological conditions, thereby enabling inference of underlying biological states.

In some embodiments, the decoding module 206 is configured to generate somatic imprints by associating the decoded physiological conditions with spatial, anatomical, or system-level representations of the one or more organs or biological systems. The somatic imprints may represent localized or distributed patterns of physiological deviation, including those influenced by sustained emotional or neurological conditions. In some embodiments, the decoding module 206 is further configured to update and refine the generated digital representations based on newly acquired biological data, thereby improving the accuracy and resolution of the physiological conditions and somatic imprints over time.

In some embodiments, the at least one processor 200 is further configured to correlate, via the correlation module 208 communicatively coupled to the at least one processor 200, the somatic imprints with emotional, neurological, and behavioral states of the user by applying the one or more artificial intelligence (AI) models 204 configured to infer relationships between physiological representations and emotional states, and generate a quantified emotional load profile based on the determined relationships. The correlation may include analyzing the somatic imprints, which represent localized or system-level physiological deviations, to identify corresponding emotional and neurological patterns associated with the user.

In some embodiments, the at least one processor 200 is configured to apply the one or more artificial intelligence models 204 to identify relationships between physiological conditions of the one or more organs or biological systems and emotional, neurological, and behavioral states. The one or more artificial intelligence models 204 may be trained using datasets comprising associations between biofrequency patterns, autonomic nervous system responses, and emotional conditions, thereby enabling inference of emotional states from the somatic imprints. In some embodiments, the correlation includes identifying patterns associated with emotional states including stress, anxiety, depressive states, heightened arousal, reduced regulatory capacity, or other emotional or neurological conditions, wherein such states are inferred based on physiological representations encoded in the somatic imprints. The correlation may further account for interactions between physiological conditions and autonomic nervous system activity, thereby linking physiological dysregulation with emotional and behavioral manifestations. The correlation module 208 forms part of an artificial intelligence-based interpretation layer configured to infer relationships between physiological representations and emotional, neurological, and behavioral states.

In some embodiments, the at least one processor 200 is configured to generate the quantified emotional load profile as a structured representation of the emotional and neurological burden associated with the user. The quantified emotional load profile may comprise numerical values, weighted scores, multi-dimensional vectors, or probabilistic measures representing the intensity, distribution, and relative contribution of different emotional states across the one or more organs or biological systems. The quantified emotional load profile may further include spatial and temporal components corresponding to localized or time-varying emotional load. In some embodiments, the quantified emotional load profile provides an integrated representation of the interaction between physiological conditions and emotional states, thereby enabling identification of systemic patterns of dysregulation arising from sustained emotional or neurological influences.

In some embodiments, the correlation module 208 is configured to correlate the somatic imprints with emotional, neurological, and behavioral states of the user and to generate the quantified emotional load profile. The correlation module 208 may comprise one or more software components, hardware components, or a combination thereof, executed by the at least one processor 200. In some embodiments, the correlation module 208 is configured to utilize the one or more artificial intelligence models 204 to infer relationships between physiological representations and emotional states, including identification of patterns linking organ-level physiological conditions with corresponding emotional or neurological conditions. The correlation module 208 may further be configured to integrate multi-domain data, including physiological data, autonomic nervous system indicators, and behavioral patterns, to generate a comprehensive emotional load representation.

In some embodiments, the correlation module 208 is configured to generate the quantified emotional load profile by aggregating and weighting inferred emotional states, thereby producing a structured representation of emotional burden across the one or more organs or biological systems. The correlation module 208 may further be configured to update the quantified emotional load profile based on newly acquired biological data, thereby enabling dynamic tracking of emotional and physiological interactions over time.

In some embodiments, the at least one processor 200 is further configured to map, via the mapping module 210 communicatively coupled to the at least one processor 200, the physiological conditions and the quantified emotional load profile to corresponding reference parameters within the reference data indicative of healthy and pathological states, by applying the one or more AI models 204 configured to learn relationships between the physiological conditions, emotional load factors, and the reference parameters. The mapping may include selecting, aligning, and associating specific elements of the physiological conditions and the quantified emotional load profile with relevant reference parameters, such that subsequent evaluation is performed using contextually appropriate reference criteria. In some embodiments, the mapping module 210 operates as part of a personalization engine configured to dynamically select and weight reference parameters based on inferred physiological conditions and quantified emotional load profile. In some embodiments, the at least one processor 200 is configured to perform multi-modal data fusion by integrating data from the plurality of sensing and analytical modalities into a unified representation, thereby enabling comprehensive interpretation of physiological and emotional conditions.

In some embodiments, the at least one processor 200 is configured to apply the one or more artificial intelligence models 204 to determine relationships between multi-dimensional inputs comprising physiological conditions and quantified emotional load factors, and corresponding parameters within the reference data. The one or more artificial intelligence models 204 may be trained to learn associations between combinations of physiological signals, emotional states, and known healthy or pathological conditions, thereby enabling identification of relevant subsets of reference parameters for a given user state. In some embodiments, the one or more artificial intelligence models 204 are configured to perform feature relevance determination, parameter selection, and weighting of reference parameters based on the combined influence of physiological conditions and emotional load factors. The mapping may therefore include assigning weights or confidence scores to different reference parameters, prioritizing certain parameters over others, and dynamically selecting those reference parameters that are most indicative of the current system state of the user.

In some embodiments, the one or more artificial intelligence models 204 are further configured to identify complex, non-linear relationships between physiological conditions, emotional load factors, and reference parameters, including interactions across multiple organ systems and regulatory pathways. The mapping may therefore account for interdependencies between physiological and emotional domains, enabling context-aware alignment of the input data with the reference data. In some embodiments, the mapping performed by the at least one processor 200 results in generation of mapped representations, wherein the physiological conditions and the quantified emotional load profile are transformed into a reference-aligned format corresponding to selected reference parameters. The mapped representations may comprise structured datasets linking specific physiological conditions and emotional load factors to corresponding healthy or pathological reference ranges.

In some embodiments, the one or more artificial intelligence models 204 are configured to update the learned relationships over time based on newly acquired biological data, thereby enabling adaptive refinement of the mapping process and improving accuracy of reference parameter selection for subsequent evaluations. In some embodiments, the mapping module 210 is configured to map the physiological conditions and the quantified emotional load profile to corresponding reference parameters within the reference data indicative of healthy and pathological states. The mapping module 210 may comprise one or more software components, hardware components, or a combination thereof, executed by the at least one processor 200.

In some embodiments, the mapping module 210 is configured to utilize the one or more artificial intelligence models 204 to perform adaptive selection and alignment of reference parameters based on learned relationships between physiological conditions, emotional load factors, and reference data. The mapping module 210 may further be configured to determine relevance, assign weights, and prioritize reference parameters to ensure that subsequent evaluation is performed using the most appropriate comparison criteria. In some embodiments, the mapping module 210 is configured to integrate multi-domain inputs, including physiological conditions and quantified emotional load profile, into a unified reference framework, thereby enabling context-aware comparison with healthy and pathological states. The mapping module 210 may further be configured to generate mapped representations for use by the analysis engine 212.

In some embodiments, the mapping module 210 is further configured to update and refine the mapping process based on feedback from subsequent evaluations and newly acquired biological data, thereby enabling continuous adaptation and improvement of reference parameter selection. In some embodiments, the at least one processor 200 is further configured to evaluate, via the analysis engine 212 communicatively coupled to the at least one processor 200, the physiological conditions and the quantified emotional load profile mapped to the corresponding reference parameters against reference data indicative of healthy and pathological states to determine system-level imbalances comprising at least one of inflammation, stress response, metabolic efficiency, and recovery capacity.

The evaluation may include comparing the mapped physiological conditions and emotional load factors with corresponding reference parameters to identify deviations from baseline or optimal regulatory states associated with the one or more tissues, one or more organs, or biological systems. In some embodiments, the at least one processor 200 is configured to determine system-level imbalances by identifying variations in frequency characteristics, spectral distributions, and physiological indicators relative to the reference data. Such variations may correspond to dysregulation in biological systems, including elevated inflammatory activity, altered autonomic nervous system balance, impaired metabolic function, or reduced capacity for physiological recovery.

In some embodiments, the determination of inflammation may include identifying signal patterns indicative of tissue-level stress responses, immune activation, or deviations in bioelectrical characteristics associated with inflammatory processes. The determination of stress response may include analysis of signal characteristics associated with autonomic nervous system activity, including sympathetic activation, parasympathetic suppression, or imbalances in regulatory states reflected in the biological data. In some embodiments, the determination of metabolic efficiency may include evaluating signal characteristics associated with cellular energy utilization, electrochemical activity, and tissue-level metabolic function inferred from the physiological conditions and corresponding digital representations. The determination of recovery capacity may include assessing the ability of the user's physiological systems to return to baseline states following perturbations, as indicated by temporal patterns, variability, and regulatory responses in the biological data.

In some embodiments, the evaluation integrates both physiological conditions and the quantified emotional load profile, thereby enabling identification of system-level imbalances arising from interactions between physiological and emotional factors. The evaluation may therefore account for compounded effects of physiological dysregulation and emotional burden on the overall functional state of the user. In some embodiments, the at least one processor 200 is further configured to generate evaluation outputs comprising one or more indicators, scores, or classifications representing the identified system-level imbalances, which are used for subsequent processing by the protocol generation module 214.

In some embodiments, the analysis engine 212 is configured to evaluate the mapped physiological conditions and the quantified emotional load profile against reference data indicative of healthy and pathological states to determine system-level imbalances. The analysis engine 212 may comprise one or more software components, hardware components, or a combination thereof, executed by the at least one processor 200. In some embodiments, the analysis engine 212 is configured to perform comparison, deviation analysis, and classification of the mapped representations relative to the reference data, thereby identifying degrees of deviation from healthy or regulated states. The analysis engine 212 may further be configured to integrate multi-domain inputs, including physiological conditions and emotional load factors, to generate system-level insights into the functional state of the user.

In some embodiments, the analysis engine 212 is configured to generate outputs representing system-level imbalances, including quantitative scores, categorical classifications, or multi-dimensional representations of inflammation, stress response, metabolic efficiency, and recovery capacity. The analysis engine 212 may further be configured to provide these outputs to the protocol generation module 214 for generation of personalized intervention plans. In some embodiments, the analysis engine 212 is further configured to update evaluation outcomes based on newly acquired biological data and refined mappings generated by the mapping module 210, thereby enabling continuous and adaptive assessment of the user's physiological and emotional state.

Further, the at least one processor 200 is configured to generate, via the protocol generation module 214 communicatively coupled to the at least one processor 200, a personalized intervention plan based on the determined system-level imbalances. The personalized intervention plan may comprise a structured set of intervention protocols tailored to the physiological conditions and the quantified emotional load profile of the user, wherein the intervention protocols are configured to address the identified system-level imbalances in a targeted and adaptive manner. In some embodiments, the at least one processor 200 is configured to select and organize one or more intervention modalities based on the type, severity, and distribution of the system-level imbalances.

The intervention modalities may include, without limitation, biochemical interventions, nutritional supplementation, compounded formulations comprising multiple ingredients, peptide-based therapies, neuromodulation techniques, behavioral modifications, activity and lifestyle restructuring, and mind-body or somatic therapies configured to regulate physiological and emotional states of the user. In some embodiments, the personalized intervention plan is configured to address both physiological and emotional components of the system-level imbalances by integrating multi-domain interventions targeting biological regulation, neurological function, and emotional patterning. The intervention plan may therefore include protocols directed toward reducing inflammation, improving metabolic efficiency, stabilizing autonomic nervous system activity, enhancing recovery capacity, and modifying emotional and behavioral response patterns. The protocol generation module 214 forms part of a personalization engine configured to generate individualized intervention strategies based on outputs of the signal processing pipeline and artificial intelligence models.

In some embodiments, the at least one processor 200 is configured to structure the personalized intervention plan in a sequential or phased manner, wherein the interventions are arranged according to a progression of system regulation and optimization. The sequential structure may include an initial phase configured to reduce physiological interference and inflammatory load, a subsequent phase configured to provide targeted biochemical and nutritional support, a further phase configured to modify neural, emotional, and behavioral response patterns, and a subsequent phase configured to enhance overall physiological and cognitive performance.

In some embodiments, the sequencing of the personalized intervention plan is determined based on the relative severity and priority of the system-level imbalances, such that interventions are applied in an order corresponding to the readiness of the user's physiological systems for regulation and recovery. The personalized intervention plan may therefore be dynamically tailored and adjusted based on the evaluated system-level imbalances and the quantified emotional load profile. In some embodiments, the personalized intervention plan further comprises actionable recommendations, schedules, dosages, and protocols configured for implementation by the user, and may be formatted for presentation via the user interface 110 or an external computing device.

In some embodiments, the protocol generation module 214 is configured to generate the personalized intervention plan based on the determined system-level imbalances. The protocol generation module 214 may comprise one or more software components, hardware components, or a combination thereof, executed by the at least one processor 200. In some embodiments, the protocol generation module 214 is configured to utilize the outputs of the analysis engine 212, including identified system-level imbalances, to select and configure appropriate intervention modalities. The protocol generation module 214 may further be configured to integrate physiological conditions and quantified emotional load factors to generate a comprehensive intervention strategy addressing both biological and emotional aspects of the user's condition.

In some embodiments, the protocol generation module 214 is configured to determine sequencing, prioritization, and progression of intervention phases, thereby enabling structured system regulation and optimization. The protocol generation module 214 may further be configured to adapt the personalized intervention plan over time based on updated system-level imbalances and feedback received from the feedback module 216. In some embodiments, the protocol generation module 214 is further configured to generate output data corresponding to the personalized intervention plan for presentation to the user via the user interface 110.

In some embodiments, the at least one processor 200 is configured to iteratively update, via the feedback module 216 communicatively coupled to the at least one processor 200, the personalized intervention plan based on subsequently acquired biological data to enable continuous recalibration of the physiological and emotional state of the user. The subsequently acquired biological data may be obtained through repeated, periodic, or continuous measurements performed by the one or more diagnostic sensors 108, thereby capturing temporal variations in the physiological conditions and the quantified emotional load profile of the user. In some embodiments, the biometric analysis system 100 operates as a closed-loop system, wherein outputs of the personalized intervention plan influence subsequently acquired biological data, and the at least one processor 200 continuously updates analysis and intervention strategies based on updated data.

In some embodiments, the at least one processor 200 is configured to process the subsequently acquired biological data in accordance with previously described operations, including extraction of signal characteristics, generation of digital representations, correlation with emotional and neurological states, mapping to reference parameters, and evaluation of system-level imbalances. The updated system-level imbalances are used to modify, refine, or reconfigure the personalized intervention plan. In some embodiments, the iterative updating includes adjusting one or more aspects of the personalized intervention plan, including modification of intervention modalities, adjustment of dosage or intensity of interventions, reordering of intervention sequences, or introduction of additional intervention protocols based on updated physiological conditions and quantified emotional load profile. The updating may reflect improvements, regressions, or newly identified patterns in the user's physiological and emotional state.

In some embodiments, the at least one processor 200 is configured to implement a closed-loop feedback mechanism, wherein execution of the personalized intervention plan influences subsequent biological data, and the subsequently acquired biological data is used to further refine the personalized intervention plan. The closed-loop system enables adaptive optimization of the intervention plan based on real-time or near real-time feedback from the user's biological system. In some embodiments, the at least one processor 200 is further configured to track longitudinal changes in the physiological conditions and the quantified emotional load profile, thereby enabling monitoring of trends associated with recovery, regulation, or progression of system-level imbalances over time. The iterative updating may therefore support progressive improvement in system performance, including reduction in inflammation, stabilization of emotional states, enhancement of metabolic efficiency, and improvement in recovery capacity.

In some embodiments, the continuous recalibration performed by the at least one processor 200 enables the personalized intervention plan to remain aligned with the evolving physiological and emotional state of the user over extended periods of time. In some embodiments, the feedback module 216 is configured to iteratively update the personalized intervention plan based on subsequently acquired biological data. The feedback module 216 may comprise one or more software components, hardware components, or a combination thereof, executed by the at least one processor 200. In some embodiments, the feedback module 216 is configured to receive updated evaluation outputs from the analysis engine 212 and to utilize such outputs to adjust the personalized intervention plan generated by the protocol generation module 214. The feedback module 216 may further be configured to coordinate re-execution of processing steps based on newly acquired biological data, thereby enabling continuous refinement of the physiological conditions and the quantified emotional load profile.

In some embodiments, the feedback module 216 is configured to implement adaptive learning mechanisms, including updating parameters, refining mappings, or adjusting weighting of reference parameters based on observed outcomes of the personalized intervention plan. The feedback module 216 may therefore contribute to improving the accuracy and effectiveness of the system over time. In some embodiments, the feedback module 216 is further configured to maintain historical records of biological data, system-level imbalances, and intervention outcomes, thereby enabling longitudinal analysis and trend-based optimization of the personalized intervention plan. The spectral components may include dominant frequency bands, harmonic structures, spectral density distributions, and frequency shifts associated with biofrequency patterns of one or more tissues or organs, while the resonance characteristics may correspond to frequency responses or oscillatory behavior indicative of tissue-specific or organ-specific physiological conditions.

In some embodiments, the waveform-based features may include amplitude variations representing signal intensity, phase relationships representing synchronization or timing differences between signal components, and temporal variations representing changes in signal characteristics over time. The waveform-based features may further include signal morphology, transient responses, and dynamic patterns associated with physiological activity, including variations related to autonomic nervous system regulation, metabolic processes, or localized tissue responses. In some embodiments, the frequency-domain features and the waveform-based features collectively represent multi-dimensional characteristics of the biological data, enabling identification of both steady-state and dynamic physiological conditions. The extracted features may capture interactions between different biological systems, including relationships between bioelectrical activity, electromagnetic responses, and physiological parameters associated with the one or more tissues, one or more organs, and cellular activity.

In some embodiments, the frequency-domain features and the waveform-based features are indicative of functional states of the user by encoding information related to physiological regulation, including autonomic balance, inflammatory activity, metabolic efficiency, and recovery capacity. The features may further reflect localized variations corresponding to specific anatomical regions or organ systems, thereby enabling downstream processing for generation of physiological conditions and somatic imprints. Further, the somatic imprints comprise data representations derived from the extracted signal characteristics that correspond to functional conditions of the one or more tissues, the one or more organs, or the biological systems of the user.

The somatic imprints may comprise structured representations including feature vectors, parameter sets, or encoded data models that capture relationships between the extracted signal characteristics and underlying physiological states. In some embodiments, the somatic imprints represent localized or system-level patterns of physiological deviation reflected in the biological data, including variations in bioelectrical activity, biofrequency patterns, and electromagnetic responses associated with the one or more tissues, the one or more organs, or the biological systems. Such patterns may indicate functional conditions including inflammation, metabolic imbalance, autonomic nervous system dysregulation, or reduced recovery capacity.

In some embodiments, the somatic imprints are derived by associating spatial or regional signal characteristics with corresponding anatomical regions, thereby enabling mapping of physiological conditions to specific tissues or organs. The somatic imprints may therefore include location-specific representations corresponding to different regions of the body, including representations associated with fascial structures or interconnected tissue networks. In some embodiments, the somatic imprints further encode patterns associated with persistent or historical physiological conditions, including those influenced by sustained emotional or neurological states. The somatic imprints may therefore capture interactions between physiological conditions and emotional factors, providing an intermediate representation for subsequent correlation with emotional, neurological, and behavioral states of the user.

In some embodiments, the somatic imprints provide a multi-dimensional representation of the functional state of the user by integrating frequency-domain features and waveform-based features into a unified data representation. The somatic imprints may therefore serve as an intermediate layer between the extracted signal characteristics and higher-level analysis performed by the correlation module 208, the mapping module 210, and the analysis engine 212. Further, the personalized intervention plan comprises a multi-stage framework for modifying the physiological and emotional state of the user. The multi-stage framework comprises a reset phase configured to reduce inflammatory load and physiological interference signals, a replenish phase configured to provide personalized biochemical and nutritional support, a regulate phase configured to modify neural and emotional response patterns, and a performance phase configured to enhance physiological and cognitive function. In some embodiments, the system performs emotional-somatic mapping by correlating signal-derived physiological representations with emotional and behavioral indicators using one or more trained artificial intelligence models.

In some embodiments, the multi-stage framework is generated by the at least one processor 200 via the protocol generation module 214 based on the determined system-level imbalances and the quantified emotional load profile, wherein the stages are arranged in a sequence corresponding to progressive regulation and optimization of the user's biological system. In some embodiments, the reset phase is configured to reduce biological noise, inflammatory load, and physiological interference signals that may impair accurate regulation of the user's system. The reset phase may include interventions directed toward detoxification, reduction of microbial load, modulation of inflammatory responses, and restoration of baseline physiological conditions, thereby preparing the user's system for subsequent intervention stages.

In some embodiments, the replenish phase is configured to provide personalized biochemical and nutritional support based on the physiological conditions and the quantified emotional load profile. The replenish phase may include administration of tailored formulations comprising one or more compounds, including vitamins, minerals, amino acids, botanicals, or nootropic agents, as well as peptide-based therapies or other biochemical interventions, thereby supporting metabolic function, cellular activity, and systemic recovery. In some embodiments, the regulate phase is configured to modify neural, emotional, and behavioral response patterns associated with the quantified emotional load profile. The regulate phase may include interventions directed toward modulation of autonomic nervous system activity, neural retraining, somatic therapies, and behavioral adjustments, thereby addressing underlying emotional and neurological factors contributing to physiological dysregulation.

In some embodiments, the performance phase is configured to enhance physiological and cognitive function of the user following regulation of the system. The performance phase may include interventions directed toward improving physical performance, cognitive function, resilience, and recovery capacity, including advanced optimization protocols, regenerative approaches, or performance-enhancing strategies. In some embodiments, the multi-stage framework is dynamically adjusted by the at least one processor 200 based on subsequently acquired biological data, such that progression between stages, duration of each stage, and specific intervention protocols are adapted according to the evolving physiological and emotional state of the user.

In some embodiments, the replenish phase comprises generating a personalized supplementation formulation including a combination of vitamins, minerals, botanicals, and nootropic compounds based on the system-level imbalances, wherein the personalized supplementation formulation comprises between 60 and 90 compounds. Further, the regulate phase comprises one or more structured neural retraining sessions configured to modify subconscious emotional and behavioral patterns of the user, wherein the structured neural retraining sessions comprise a predefined sequence of sessions conducted over a plurality of time intervals.

Further, the at least one processor 200 using the user interface 110 operatively coupled to the at least one processor 200, is configured to present the physiological conditions, the quantified emotional load profile, and the personalized intervention plan, where the user interface 110 is executed on at least one of the device 106 or an external computing device communicatively coupled to the biometric analysis system 100. The external computing device may include, without limitation, a mobile device, tablet, or computer system configured to receive output data generated by the at least one processor 200.

In some embodiments, the at least one processor 200 is configured to generate output data corresponding to the physiological conditions, the quantified emotional load profile, and the personalized intervention plan, and to transmit the output data to the user interface 110 for presentation. The physiological conditions may be presented as visual, numerical, or graphical representations of functional states of the one or more tissues, one or more organs, or biological systems, including indicators of system-level imbalances such as inflammation, stress response, metabolic efficiency, and recovery capacity.

In some embodiments, the quantified emotional load profile is presented as a structured representation comprising scores, distributions, or graphical indicators representing the intensity and spatial distribution of emotional and neurological states across the one or more organs or biological systems. The presentation may include multi-dimensional views of emotional load, including temporal variations and localized concentrations associated with specific anatomical regions. In some embodiments, the personalized intervention plan is presented as a structured set of actionable recommendations, schedules, dosages, and protocols, including indications of sequencing, priority, and progression of intervention phases. The presentation may further include guidance for implementation, progress tracking indicators, and updates corresponding to iterative recalibration performed by the feedback module 216.

In some embodiments, the user interface 110 is configured to support interactive visualization, including selection of specific organs, regions, or system-level imbalances, and display of detailed information associated with selected elements. The user interface 110 may further enable navigation between different views, including summary views, detailed diagnostic views, and intervention planning views. In some embodiments, the at least one processor 200 is configured to update the presented physiological conditions, quantified emotional load profile, and personalized intervention plan in real time or near real time based on subsequently acquired biological data, thereby enabling continuous monitoring and visualization of changes in the user's physiological and emotional state.

In some embodiments, the user interface 110 may further integrate the color spectrum corresponding to entropy values and the three-dimensional representations of organs or biological systems, as described herein, to provide an intuitive and spatially resolved visualization of system-level imbalances and emotional load. Further, the at least one processor 200 using the user interface 110 is further configured to present the physiological conditions and the quantified emotional load profile using a color spectrum corresponding to entropy values associated with the physiological conditions or the quantified emotional load profile. In some embodiments, the color spectrum comprises a gradient ranging from lighter colors representing lower entropy values to darker colors representing higher entropy values.

In some embodiments, the at least one processor 200 is configured to determine the entropy values based on variations in the mapped physiological conditions and the quantified emotional load profile relative to the reference data. The entropy values may be derived from statistical measures, spectral dispersion, signal variability, or multi-dimensional deviations associated with the biological data, thereby providing an indicator of system-level imbalance. In some embodiments, the color spectrum comprises a gradient ranging from lighter colors representing lower entropy values to darker colors representing higher entropy values. The lighter colors may correspond to states of higher physiological coherence, stability, or regulation, while the darker colors may correspond to states of increased physiological disruption, imbalance, or elevated emotional load. The gradient may further include intermediate color transitions to represent varying degrees of deviation across the physiological conditions and the quantified emotional load profile.

In some embodiments, the at least one processor 200 is configured to apply the color spectrum to one or more visual representations presented via the user interface 110, including graphical displays, charts, or anatomical representations of the one or more organs or biological systems. The color mapping may be applied globally or locally, including region-specific mapping corresponding to particular tissues or organs, thereby enabling spatial visualization of entropy values and facilitating identification of areas associated with higher system-level imbalances. In some embodiments, the color-based representation enables intuitive interpretation of the physiological conditions and the quantified emotional load profile by visually conveying severity, distribution, and progression of system-level imbalances. The at least one processor 200 may further be configured to dynamically update the color spectrum representation based on subsequently acquired biological data, thereby reflecting changes in entropy values over time.

Further, the at least one processor 200 using the user interface 110 is further configured to present a three-dimensional representation of one or more organs or biological systems of the user, and to display one or more visual markers on the three-dimensional representation corresponding to locations of the physiological conditions or system-level imbalances. In some embodiments, the at least one processor 200 is configured to generate the three-dimensional representation based on anatomical models, digital organ templates, or user-specific representations of the one or more organs or biological systems, thereby providing a spatially resolved visualization environment.

In some embodiments, the at least one processor 200 is configured to map the physiological conditions and the system-level imbalances to corresponding anatomical locations within the three-dimensional representation based on the somatic imprints and spatial characteristics of the biological data. The mapping may include associating localized signal characteristics, regional biofrequency deviations, or organ-specific physiological conditions with corresponding regions of the three-dimensional model. In some embodiments, the one or more visual markers comprise graphical indicators, overlays, color-coded regions, or symbols displayed on the three-dimensional representation to indicate the presence, location, and relative severity of the physiological conditions or system-level imbalances. The visual markers may vary in size, intensity, shape, or color based on the magnitude or significance of the corresponding imbalance.

In some embodiments, the visual markers are presented in combination with the color spectrum corresponding to entropy values, such that the color of each visual marker or region reflects the associated entropy value of the physiological conditions or the quantified emotional load profile. This enables simultaneous visualization of both spatial distribution and severity of system-level imbalances across the one or more organs or biological systems. In some embodiments, the user interface 110 is configured to enable interaction with the three-dimensional representation, including rotation, zooming, selection of specific organs or regions, and display of additional information associated with selected visual markers. The additional information may include corresponding physiological conditions, quantified emotional load values, and relevant portions of the personalized intervention plan associated with the selected region.

In some embodiments, the at least one processor 200 is configured to dynamically update the three-dimensional representation and the associated visual markers based on subsequently acquired biological data, thereby enabling continuous visualization of changes in physiological conditions and system-level imbalances over time. In some embodiments, the analysis engine 212 is further configured to compare spectral characteristics of the biological data with reference spectral data corresponding to healthy tissue and pathological conditions to determine a degree of similarity and identify a corresponding pathological tendency. The spectral characteristics may include frequency components, spectral density distributions, resonance peaks, harmonic structures, and frequency deviations derived from the biological data.

In some embodiments, the analysis engine 212 is configured to perform spectral matching between the spectral characteristics of the biological data and the reference spectral data associated with known healthy and pathological states of one or more tissues, one or more organs, or biological systems. The comparison may include calculating similarity measures, correlation coefficients, distance metrics, or pattern-matching scores indicative of the degree of similarity between the observed spectral characteristics and the reference spectral data. In some embodiments, the degree of similarity may be represented as a quantitative score, probability value, or ranking indicating the closeness of the biological data to one or more reference conditions. The analysis engine 212 may be configured to identify one or more reference conditions having the highest degree of similarity and to associate such reference conditions with corresponding pathological tendencies or physiological states.

In some embodiments, the identified pathological tendency may correspond to a likelihood or predisposition toward a particular physiological condition, including inflammatory processes, metabolic dysfunction, stress-related conditions, or other system-level imbalances. The pathological tendency may therefore represent an inferred direction or trend of physiological change based on similarity of spectral characteristics.

In some embodiments, the analysis engine 212 is further configured to incorporate the spectral comparison results into the determination of system-level imbalances, thereby enhancing the accuracy and specificity of evaluation performed by the analysis engine 212. The spectral comparison may be performed in conjunction with the mapped physiological conditions and the quantified emotional load profile, enabling integration of spectral similarity with multi-domain analysis. In some embodiments, the analysis engine 212 may be further configured to update the reference spectral data and similarity relationships based on newly acquired biological data and observed outcomes, thereby improving the robustness and accuracy of pathological tendency identification over time.

In some embodiments, the somatic imprints comprise data representations corresponding to functional conditions of the one or more organs or biological systems derived from the extracted signal characteristics. Further, the quantified emotional load profile comprises weighted indicators corresponding to stress, emotional reactivity, and behavioral patterns of the user. In some embodiments, the analysis engine 212 is configured to evaluate the physiological conditions and the quantified emotional load profile as a combined multi-domain model for determining the system-level imbalances. The combined multi-domain model may comprise an integrated representation in which physiological parameters and emotional load factors are jointly analyzed to capture interactions between biological, neurological, and behavioral domains of the user.

In some embodiments, the analysis engine 212 is configured to construct the combined multi-domain model by integrating the mapped physiological conditions and the quantified emotional load profile into a unified analytical framework, wherein each domain contributes to the overall assessment of system-level imbalances. The integration may include combining feature sets, aligning domain-specific parameters, and establishing relationships between physiological indicators and emotional states. In some embodiments, the analysis engine 212 is configured to account for interdependencies between physiological conditions and emotional load factors, including the influence of emotional stress on autonomic nervous system activity, metabolic function, and inflammatory responses, and the reciprocal effects of physiological dysregulation on emotional and behavioral states. The combined multi-domain model may therefore capture bidirectional interactions between physiological and emotional domains.

In some embodiments, the analysis engine 212 is configured to evaluate the combined multi-domain model using weighted contributions from the physiological conditions and the quantified emotional load profile, wherein the weights may be determined based on relevance, severity, or contextual importance of each domain. The evaluation may therefore reflect a composite assessment of system-level imbalance that accounts for both physiological and emotional factors. In some embodiments, the combined multi-domain model enables the analysis engine 212 to identify system-level imbalances that may not be detectable when evaluating physiological conditions or emotional states independently, thereby improving accuracy and contextual relevance of the evaluation. In some embodiments, the analysis engine 212 is further configured to update the combined multi-domain model based on subsequently acquired biological data and updated quantified emotional load profiles, thereby enabling dynamic refinement of the integrated analysis over time.

In some embodiments, the biological data acquired by the one or more diagnostic sensors 108 further comprises signal characteristics associated with fascial tissue of the user. The at least one processor 200 may be configured to analyze electromagnetic responses corresponding to fascial structures, wherein the fascial tissue exhibits piezoelectric properties such that mechanical deformation, tension, or restriction within the fascial network produces measurable electrical signals. In some embodiments, the at least one processor 200 is configured to identify localized variations in the electromagnetic excitation signals corresponding to regions of fascial restriction or contraction, wherein such variations represent somatic encoding of physiological or emotional stress within the connective tissue matrix. The identified fascial signal patterns may be incorporated into the physiological conditions and the somatic imprints representing functional states of the one or more organs or biological systems.

In some embodiments, the at least one processor 200 is further configured to determine autonomic nervous system (ANS) states of the user based on the biological data, wherein the ANS states comprise at least one of a parasympathetic-dominant state, a sympathetic activation state, or a reduced regulatory or shutdown state. The determination may be based on analysis of signal characteristics including heart rate variability patterns, frequency distributions, and temporal signal variations indicative of autonomic regulation. In some embodiments, the determined ANS states are incorporated into the physiological conditions and the quantified emotional load profile, such that the correlation and evaluation steps account for autonomic signatures associated with chronic emotional states and regulatory capacity of the user.

In some embodiments, the at least one processor 200 is configured to determine spatial or regional characteristics of the biological data, wherein the biological data is associated with distinct anatomical regions comprising at least one of thoracic, abdominal, pelvic, or cervical regions of the user. The at least one processor 200 may be configured to generate region-specific physiological conditions and somatic imprints corresponding to localized signal characteristics. In some embodiments, the regional characteristics are used to enhance spatial mapping of system-level imbalances and emotional load, such that the correlation module and the analysis engine 212 are configured to identify region-specific dysregulation within the one or more organs or biological systems.

In some embodiments, the at least one processor 200 is further configured to associate the physiological conditions and the somatic imprints with predefined organ-emotion relationships, wherein each of the one or more organs or biological systems is associated with physiological activity of one or more corresponding emotional patterns. The association may include mapping liver-related physiological conditions to emotional states comprising anger or frustration, lung-related physiological conditions to emotional states comprising grief or sadness, kidney-related physiological conditions to emotional states comprising fear or anxiety, heart-related physiological conditions to emotional states comprising emotional dysregulation or shock, and digestive system-related physiological conditions to emotional states comprising worry or cognitive overactivity. In some embodiments, the predefined organ-emotion relationships are stored as part of the reference data and are used by the correlation module to enhance inference of emotional, neurological, and behavioral states of the user.

In some embodiments, the at least one processor 200 is configured to generate a structured representation of the physiological conditions, the somatic imprints, and the quantified emotional load profile, wherein the structured representation comprises a somatic-emotional blueprint of the user. The somatic-emotional blueprint may include a system-level and region-specific representation of physiological dysregulation, associated emotional patterns, and hierarchical organization of system-level imbalances. In some embodiments, the somatic-emotional blueprint is used by the protocol generation module 214 to determine sequencing, prioritization, and selection of intervention protocols.

In some embodiments, the at least one processor 200 is configured to determine a release sequence based on relative severity, depth, or persistence of the system-level imbalances and the quantified emotional load profile. The release sequence may represent an ordered progression of intervention targeting, wherein system-level imbalances associated with higher regulatory readiness or lower structural depth are addressed prior to imbalances associated with deeper or more persistent physiological or emotional encoding.

In some embodiments, the personalized intervention plan comprises a plurality of intervention modalities organized into multiple domains comprising at least one of a consciousness-based intervention domain, a neuroscience-based intervention domain, a movement or somatic intervention domain, and a biochemical or regenerative intervention domain. The at least one processor 200 may be configured to select and coordinate intervention modalities across the plurality of domains based on the somatic-emotional blueprint and the determined system-level imbalances.

In some embodiments, the biological data further comprises electromagnetic field characteristics associated with a biofield of the user, wherein the biofield represents an electromagnetic field generated by physiological activity of the user. The at least one processor 200 may be configured to analyze variations in the electromagnetic field characteristics to identify perturbations associated with physiological or emotional dysregulation, and to incorporate such perturbations into the somatic imprints and the quantified emotional load profile.

In some embodiments, the at least one processor 200 is further configured to determine a system performance metric of the user based on the physiological conditions and the quantified emotional load profile, wherein the system performance metric represents an overall functional efficiency of the user's biological system. The system performance metric may be indicative of combined physiological regulation, emotional stability, metabolic efficiency, and recovery capacity, and may be used to assess improvement over time as part of the iterative updating of the personalized intervention plan. In some embodiments, the at least one processor 200 is configured to determine a recovery metric corresponding to a rate at which the physiological conditions and the quantified emotional load profile return toward baseline or regulated states following a perturbation or stress event. The recovery metric may be derived from temporal variations in the biological data and may be used as a primary indicator of system resilience and regulatory capacity.

In some embodiments, the personalized intervention plan is configured as a longitudinal protocol extending over a predefined duration, wherein the protocol comprises a plurality of sequential intervention stages distributed over the predefined duration. The predefined duration may correspond to a long-term intervention period, and the personalized intervention plan may be updated periodically to generate stage-wise action plans based on subsequently acquired biological data. In some embodiments, the personalized intervention plan comprises a plurality of periodic action plans corresponding to defined time intervals, wherein each action plan comprises a subset of intervention protocols tailored to the physiological conditions and the quantified emotional load profile at a corresponding time interval. The periodic action plans may be generated and updated iteratively based on changes in system-level imbalances.

In some embodiments, the personalized intervention plan comprises a personalized formulation comprising a plurality of compounds selected based on the physiological conditions and the quantified emotional load profile. The plurality of compounds may include one or more of vitamins, minerals, amino acids, botanical extracts, or nootropic agents, wherein the formulation comprises a combination of multiple compounds configured to target system-level imbalances across multiple biological systems. In some embodiments, the personalized intervention plan further comprises one or more behavioral or lifestyle modification protocols configured to alter daily activity patterns, sleep behavior, dietary habits, or physical activity of the user, wherein such modifications are selected based on the physiological conditions and the quantified emotional load profile.

In some embodiments, the personalized intervention plan comprises one or more structured neural retraining sessions configured to modify neural activity patterns associated with the quantified emotional load profile, wherein the neural retraining sessions are configured to reduce maladaptive stress responses, modify subconscious behavioral patterns, and improve emotional regulation. In some embodiments, the at least one processor 200 is configured to determine that system-level imbalances associated with physiological dysregulation may limit effectiveness of external interventions, wherein the personalized intervention plan is configured to prioritize regulation of the physiological conditions prior to implementation of performance-enhancing or outcome-specific interventions.

In some embodiments, the personalized intervention plan is continuously recalibrated based on iteratively acquired biological data, wherein the recalibration comprises adjusting intervention protocols to maintain alignment with evolving physiological conditions, quantified emotional load profile, and system performance metrics of the user. In some embodiments, the personalized intervention plan is configured to progressively improve the self-regulatory capacity of the user's biological system, such that reliance on external interventions is reduced over time as the physiological conditions and emotional regulation approach stable baseline states.

In some embodiments, the at least one processor 200 is configured to model interactions between multiple domains comprising physiological conditions, emotional states, neurological activity, and behavioral patterns of the user, wherein the interactions between the multiple domains are analyzed as an integrated system. The at least one processor 200 may be configured to determine cross-domain influences, including effects of emotional states on physiological conditions and reciprocal effects of physiological dysregulation on behavioral and neurological states, thereby enabling unified system-level evaluation. In some embodiments, the personalized intervention plan is implemented according to a structured multi-stage protocol comprising a homeostasis method including a plurality of sequential stages comprising a reset stage, a replenish stage, a regulate stage, and a performance stage, wherein the stages collectively operate to restore and optimize system-level balance of the user.

In some embodiments, the physiological conditions further comprise indicators associated with gut-brain axis function and microbiome-associated physiological states, wherein the at least one processor 200 is configured to identify patterns indicative of imbalances associated with digestive function, gut-related physiological activity, and signaling influencing neurological and emotional states, based on the biological data. The identified gut-brain axis conditions may be incorporated into the system-level imbalances and the personalized intervention plan. In some embodiments, the physiological conditions comprise indicators associated with toxin load, microbial imbalance, or presence of pathogenic factors including parasites, fungal activity, or environmental toxins. The at least one processor 200 may be configured to infer such conditions based on deviations in the biological data and incorporate corresponding detoxification or clearance protocols within the personalized intervention plan.

In some embodiments, the correlation module 208 is configured to perform emotional-somatic decoding, wherein somatic imprints derived from the biological data are analyzed to identify underlying emotional patterns, subconscious behavioral drivers, and nervous-system dysregulation associated with the user. In some embodiments, the physiological conditions comprise indicators associated with toxin load, microbial imbalance, or presence of pathogenic factors including parasites, fungal activity, or environmental toxins. The at least one processor 200 may be configured to infer such conditions based on deviations in the biological data and incorporate corresponding detoxification or clearance protocols within the personalized intervention plan.

In some embodiments, the correlation module 208 is configured to perform emotional-somatic decoding, wherein somatic imprints derived from the biological data are analyzed to identify underlying emotional patterns, subconscious behavioral drivers, and nervous-system dysregulation associated with the user. In some embodiments, the personalized intervention plan further comprises advanced regenerative interventions including one or more of peptide-based therapies, stem cell-based therapies, exosome-based therapies, or mitochondrial and metabolic enhancement protocols, wherein such interventions are selected based on the determined system-level imbalances and system performance metrics.

In some embodiments, the biological data is acquired from a plurality of data sources comprising at least one of sensor-based electromagnetic measurements, laboratory-based biological assessments, physiological monitoring systems, or external diagnostic platforms. The plurality of data sources may include subclinical biofeedback data, blood-based biomarker data, frequency-based signal data, emotional somatic mapping data, and cardio-metabolic health data, wherein the at least one processor 200 is configured to integrate the plurality of data sources to generate the physiological conditions and the quantified emotional load profile.

In some embodiments, the biometric analysis system 100 further comprises the device 106 configured to generate a feedback signal configured to be applied to the user. The feedback signal may comprise one or more electromagnetic, optical, or frequency-based output signals configured to interact with the user's biological system. The device 106 may be configured to deliver such signals in proximity to or in contact with the user to influence physiological or neurological states. In some embodiments, the at least one processor 200 is configured to determine the feedback signal in accordance with the personalized intervention plan, wherein the feedback signal is selected based on the determined system-level imbalances, physiological conditions, and quantified emotional load profile. The feedback signal may therefore form part of the personalized intervention plan and may be applied to support regulation, recovery, and optimization of the user's biological system.

In some embodiments, the feedback signal comprises one or more of electromagnetic field signals, pulsed electromagnetic excitation signals, light-based signals including red or near-infrared light, or frequency-based signals configured to influence cellular activity, neurological function, or systemic regulation of the user. The feedback signal may be configured to support processes including cellular energy production, circulation, nervous system regulation, and recovery. In some embodiments, the device 106 comprises a wearable device, a handheld device, or a stationary device configured to deliver the feedback signal to the user. The wearable device may comprise a frequency-emitting device configured to provide continuous or periodic signals, while the stationary device may comprise therapeutic systems configured to deliver electromagnetic or light-based stimulation. In some embodiments, the feedback signal is configured to modulate at least one physiological or neurological state of the user, including autonomic nervous system activity, cellular energy production, metabolic function, or emotional regulation, without requiring invasive intervention.

In some embodiments, the biometric analysis system 100 is configured to orchestrate the plurality of sensing modalities, data processing operations, artificial intelligence models, and intervention generation processes into a unified system, wherein the orchestration enables transformation of raw biological signal data into structured, interpretable, and actionable outputs.

At step 302, the biometric analysis system 100 is configured to eliminate internal and external stressors from the user to bring the body to an optimal state of performance, in the detox and optimization phase. In some embodiments, first phase of the method is directed toward bringing the human body (i.e. user body) to an optimal state of performance by eliminating internal and external stressors that interfere with physiological and emotional balance. This foundational phase begins with a comprehensive detoxification process designed to clear the body of various biological and environmental burdens. Internal stressors targeted during this stage may include parasites, viruses, harmful bacteria, and fungi, all of which can impair immune function, digestion, and systemic performance. External stressors such as hormonal disruptors and environmental toxins commonly encountered through food, air, water, and consumer products are also identified and addressed.

In some embodiments, the biometric analysis system 100 utilizes non-invasive diagnostic tools to detect the presence of these stressors by analyzing the wave characteristics of biological tissues. In some embodiments, the biometric analysis system 100 utilizes the One or more AI models 204 to decode bioelectrical activity and correlate specific frequencies with known physiological imbalances or toxin exposures. Once identified, personalized detoxification protocols are recommended, which may include natural remedies, frequency-based therapies, or lifestyle modifications tailored to the individual's specific condition.

Further, upon following detoxification, the biometric analysis system 100 focuses on restoring and optimizing gut health, a critical component of overall physiological function. The gut, often referred to as the “second brain,” plays a central role in regulating digestion, immune response, hormonal balance, and even emotional health. By rebalancing the gut microbiome, supporting digestion, and healing the intestinal lining, the system promotes a state of internal harmony that supports both physical and emotional resilience. This gut optimization process is informed by diagnostic findings and personalized to the unique needs of each individual. The completion of this phase prepares the body for more advanced stages of analysis and therapeutic intervention aimed at long-term health and performance optimization.

At step 304, the plurality of tests is conducted to assess a current state of the body of the user. In some embodiments, in the diagnostics & data collection phase serves as the foundation for the personalized health optimization process. In this phase, a wide-ranging series of assessments are performed to establish a precise and holistic understanding of the subject's current physiological, biochemical, and emotional health status. By integrating advanced proprietary technologies with conventional diagnostics, the system ensures a multi-layered, in-depth evaluation that addresses both visible symptoms and hidden dysfunctions.

At the core of this phase are two proprietary technologies signal-based physiological analysis system and Biodecodification. The signal-based physiological analysis system is a non-invasive investigative method based on the detection and analysis of electromagnetic wave patterns emitted by the body. This technology utilizes specialized sensors that scan and interpret subtle frequency fluctuations corresponding to various tissues, organs, and cellular activities. By reading these signals, the biometric analysis system 100 identifies disturbances in energetic balance and cellular function, often before physical symptoms appear. The signal-based physiological analysis system provides early-stage detection of dysfunctions, including inflammation, organ stress, immune irregularities, and neurochemical imbalances. The quantum diagnostic deeply integrated with artificial intelligence (AI), enabling rapid comparison with vast datasets of healthy and pathological frequencies, allowing for highly accurate pattern recognition and condition mapping.

Biodecodification, also proprietary, complements signal-based physiological analysis system by decoding the emotional and psychological imprints stored within the body's somatic systems. This technique interprets vibrational data linked to quantified emotional load profile and other inferred physiological conditions, emotional stress, or unresolved psychological conflicts, and maps them to corresponding organ systems or physiological patterns. For example, unresolved grief may manifest in the respiratory system, while chronic fear may correlate with adrenal stress. The biometric analysis system 100 applies this analysis to guide individualized therapeutic strategies that address the emotional roots of physiological disorders something traditional diagnostics typically overlook.

In addition to these technologies, the analysis process incorporates evaluation of gut-related physiological conditions and microbiome-associated functional states inferred from the biological data. The at least one processor 200 is configured to identify patterns indicative of gastrointestinal function, gut-brain axis signaling, and metabolic activity associated with the digestive system. As the gut is recognized as an important contributor to immune regulation, neurological function, and nutrient processing, gut-related physiological conditions are incorporated into the overall system-level assessment. In some embodiments, patterns indicative of gastrointestinal imbalance, including those associated with dysregulation of digestive processes or barrier function, may influence physical and emotional states and are considered in generating the personalized intervention plan. The biometric analysis system 100 also integrates comprehensive bloodwork to evaluate critical biomarkers related to metabolic function, immune activity, organ performance, inflammation levels, and nutritional deficiencies. These tests offer direct biochemical insight that complements the vibrational and frequency-based data gathered from the proprietary tools.

In some embodiments, the hormone panel is conducted to examine endocrine function, including stress-related hormones (e.g., cortisol), reproductive hormones (e.g., estrogen, progesterone, testosterone), and metabolic regulators (e.g., insulin, thyroid hormones). Hormonal imbalances can profoundly impact energy, mood, cognitive function, and metabolic efficiency. Understanding these levels enables targeted interventions to restore hormonal harmony. For structural and body composition analysis, the system may incorporate a DEXA (Dual-Energy X-ray Absorptiometry) scan, which provides high-precision data on bone density, lean muscle mass, and fat distribution. This is particularly useful for assessing risk factors for osteoporosis, metabolic syndrome, and tracking physical progress during therapy.

At step 306, the plurality of test results is aggregated and analysed to generate a personalized action plan. Following the diagnostics phase, the server 104, using the at least one processor 200, is configured to initiate a proprietary algorithmic assessment and protocol generation process. This phase is powered by a specially designed algorithm embedded within the at least one processor's software framework. The at least one processor 200 using the one or more AI models 204 is configured to aggregate, correlate, and interpret all data collected during diagnostics including quantum frequency readings, biodecodification findings, microbiome analysis, blood biomarkers, hormonal levels, and imaging results such as DEXA. By integrating these diverse datasets, the at least one processor 200 builds a comprehensive, multi-dimensional profile of the user's physiological and emotional state.

In some embodiments, the at least one processor 200 is configured to translate diagnostic data into actionable, personalized protocols. The at least one processor 200 does so by identifying patterns, correlations, and anomalies within the collected data and comparing them against a vast internal knowledge base of clinical outcomes, frequency databases, physiological models, and historical case data. Through this intelligent analysis, the biometric analysis system 100 is able to pinpoint the underlying dysfunctions whether biological, biochemical, emotional, or energetic that may be limiting the user's current performance or long-term health.

Further, once the root causes are determined, the at least one processor 200 is configured to automatically generates the personalized action plan tailored to the user's specific needs and health objectives. The protocol may include targeted interventions across multiple domains such as prevention, physiological optimization, emotional recalibration, and anti-aging strategies. For instance, the plan may recommend specific detox measures, dietary changes, frequency-based therapies, hormonal balancing, supplement regimens, and emotional support modalities. It may also highlight long-term lifestyle changes designed to enhance longevity, resilience, and cellular regeneration.

In some embodiments, the protocol generation process is dynamic and designed to adapt over time as the patient's metrics evolve. The at least one processor 200 continually reassess incoming data from follow-up scans and feedback loops, allowing for real-time updates to the protocol based on the body's response to previous interventions. This adaptive functionality ensures that the health plan remains relevant, effective, and aligned with the individual's ongoing physiological changes and optimization goals.

In one embodiment, the at least one processor 200 executes a proprietary algorithm that is employed to generate individualized action plans based on a combination of biometric test results and user-supplied contextual data. The at least one processor 200 using the One or more AI models 204 instantiates a trained artificial intelligence (AI) chatbot model. Instead, the AI chatbot model is developed through supervised machine learning (ML) techniques using training data curated by licensed physicians and healthcare professionals affiliated with the assignee entity. The One or more AI models 204 ingests multiple data streams, including but not limited to (i) vibrational frequency measurements obtained via a diagnostic scanning device, and (ii) user responses to structured health and wellness questionnaires. These questionnaires serve to contextualize biometric data by capturing emotional, cognitive, and physiological indicators that may correspond to underlying energetic imbalances or health risks.

The AI chatbot model utilizes natural language processing (NLP) and predictive modelling to correlate user-specific data with known emotional and physical pattern signatures. The AI chatbot model subsequently narrows down and ranks potential areas of concern. Based on these results, the One or more AI models 204 funnels recommendations that constitute a personalized action plan. This plan may include therapeutic suggestions, referrals to qualified specialists, lifestyle modifications, or product and service offerings available through the platform. Crucially, the One or more AI models 204 is continuously updated. It is retrained at regular intervals using anonymized feedback from previous client cases, as well as with newly introduced products and services in the company's ecosystem. This continuous learning process allows the biometric analysis system 100 to maintain clinical relevance and commercial alignment, ensuring that the personalized action plans reflect both up-to-date wellness strategies and current product availability.

At step 308, electromagnetic waves are transmitted, via one or more diagnostic sensors, to recognize specific frequencies (Vortex magnetic field) and to detect functional status of the body. At step 310, specific temporary conditions and somatic imprints of organs and tissues are gathered using the one or more diagnostic sensors. At step 312, the at least one processor 200 is configured to convert the specific temporary conditions and somatic imprints of organs and tissues to a digital sequence to quantify the user's quantified emotional load profile and other inferred physiological conditions, using the One or more AI models 204.

In some embodiments, the at least one processor 200 utilizes electromagnetic waves transmitted through the one or more diagnostic sensors 108 to detect and analyze specific vortex magnetic fields emitted by biological objects, such as tissues, cells, chromosomes, enzymes, and hormones. These vortex magnetic fields are fundamental to the communication processes within biological systems, playing a critical role in intercellular and extracellular interactions. The at least one processor 200 is designed to perform a spectral analysis of these vortex magnetic fields to monitor changes in the body's functional status.

The vortex magnetic field, identified as an extremely weak, low-frequency signal around all biological systems, facilitates the transfer of information within the body. This phenomenon has been demonstrated through extensive research conducted at the Institute, which confirmed that biological systems utilize these magnetic fields for regulating cellular and tissue-level activities. The non-linear analysis systems (NLS) are based on this research, providing a unique and powerful tool for detecting the subtle energetic imbalances within the body.

By transmitting electromagnetic waves, the one or more diagnostic sensors 108 are able to interact with the vortex magnetic fields of the body and capture their frequency characteristics. Further, the at least one processor 200 using the One or more AI models 204 is configured to analyse these frequencies to identify deviations from normal or healthy biological patterns, enabling the detection of early-stage dysfunctions or imbalances that may not be detectable using traditional diagnostic methods. The analysis of these vortex magnetic fields is considered a highly advanced method, offering a non-invasive, analogue-free means of assessing the body's internal health status, while also providing insights into emotional and psychological imbalances that may manifest at the cellular level.

In some embodiments, the at least one processor 200 is designed to facilitate the production of preset bioelectrical activity within brain neurons, which serves as a baseline or background signal for the analysis. This bioelectrical activity allows for the amplification of signals that are typically hardly detectable due to their subtle presence amidst statistical fluctuations in biological data. Once these signals are amplified, the at least one processor 200 isolates them from the noise and decodes the valuable information they contain, providing insight into the body's functional state.

Further, the at least one processor 200 takes precise measurements of the electromagnetic radiation generated by the biological systems under observation, particularly focusing on its origin. The at least one processor 200 further processes the measurements using the One or more AI models 204 to decode the information embedded in the electromagnetic fields. The decoded data is displayed on the user interface 110 and also stored in the memory 202, where a virtual model of the relevant organ is constructed. This virtual model is visually represented with specific colors that correspond to varying levels of entropy in the biological systems, following the principles of quantum chromo kinetics.

Quantum chromo kinetics involves representing the entropy values of any biological system as a spectrum of colors. The color spectrum starts from light yellow, which signifies minimum entropy values, and gradually progresses through orange, red, and purple, ending with nearly black for maximum entropy values. The changes in the color spectrum reflect variations in the energy and functional states of the biological structures being analyzed. The biometric analysis system 100 may detect subtle shifts in entropy, which may indicate areas of dysfunction or pathology within the body. This color mapping provides a visual representation of the internal conditions of the organ, offering physicians a quick and accurate means of diagnosis.

In some embodiments, after analysing the spectral characteristics of the biological system and detecting imbalances or disturbances, the at least one processor 200 is further configured to search for a remedy that matches the spectral characteristics of the detected pathological process. The at least one processor 200 identifies remedies by comparing the spectral signatures of various interventions or therapeutic substances in its database, selecting the one that most closely aligns with the disharmonic frequencies present in the body. This process is based on the understanding that disease can be conceptualized as a disturbance in the harmonic synchronization of biological systems. When the natural harmonic patterns of tissues, cells, or organs are disrupted, it creates disharmonic electromagnetic oscillations that interfere with the normal functioning of the body. These disturbances act as blockages or noise within the system, disrupting the body's natural energy flow and leading to dysfunction or disease.

The biometric analysis system 100 leverages the principle of electromagnetic resonance to correct these imbalances. By identifying the disharmonic oscillations that are causing the disturbance, the biometric analysis system 100 seeks to counteract them by applying electromagnetic oscillations with the opposite phase or inverted sign. This concept is rooted in the laws of physics, specifically the principle of destructive interference, where two opposing waveforms cancel each other out. In this case, the algebraic sum of the disharmonic oscillations and the inverted remedy frequencies would theoretically lead to a state of equilibrium, bringing the body's electromagnetic field back into harmony.

In some embodiments, the biometric analysis system 100 does not merely mask the symptoms of the disease, but instead works to correct the underlying energy imbalances by restoring harmonic synchronization at the electromagnetic level. The remedy selected by the at least one processor 200 may therefore be seen as a therapeutic counterbalance, designed to specifically target the root cause of the disturbance, thereby facilitating healing and restoration of normal physiological function. This approach represents an innovative form of treatment, where electromagnetic therapy is used to directly influence the body's bioenergetic state, providing a non-invasive and scientifically grounded method of correction. The remedy is chosen based on precise spectral matching to ensure that the intervention is as effective as possible in addressing the unique pathological characteristics identified in the diagnostic phase.

In some embodiments, the at least one processor 200 using the One or more AI models 204 is configured to operate exclusively with a telemetric nonlinear analysis data processing technique and its subsequent modifications. The at least one processor 200 using the One or more AI models 204 is designed for compatibility with standard personal computers (PCs) and facilitates the study of biological responses to various types of informational stimuli, including electromagnetic and vibrational influences. The at least one processor 200 is engineered to support correlation between the measurement process and the real-time effects of the informational impact on the subject. The at least one processor 200 performs a sequence of operations essential for capturing and analyzing the functional state of the biological system comprising measurement of J(0) parameter in which the at least one processor 200 initially measures the parameter J(0), which reflects changes in the describing parameter representing a shift in the entropic potential relative to its baseline (initial) value. This provides a quantitative foundation for assessing deviations in physiological equilibrium.

Further, sequence of operation comprises frequency transformation to histogram in which the continuous signal J(0) is transformed into a frequency histogram, segmenting the data into preset intervals across a spectral range from 1.8 Hz to 8.2 Hz. Each scanned frequency is represented as a numerical value assigned with a serial identifier, forming a digital matrix suitable for further pattern recognition and analysis. Further, sequence of operation comprises real-time visualization and impact in which the current W values representing vibrational response parameters are displayed over the user interface 110. This allows an operator to observe the physiological reactions in real time, synchronized with the application of therapeutic or diagnostic influences on the subject. Further, sequence of operation comprises data storage for post-processing in which the W values may be accumulated and stored within the memory 202 to enable review and evaluation of the histogram following the completion of active measurements. This functionality supports delayed analysis and enhances convenience during multi-phase diagnostics.

Further, sequence of operation comprises regulatory feedback control in which during testing, the at least one processor 200 is capable of issuing scale-correlated commands, allowing modulation or regulation of the informational impact on the subject. This feedback control mechanism enhances precision and safety in response modulation. Further, sequence of operation comprises final data transmission and storage in which upon completion of the measurement sequence, the at least one processor 200 transmits the recorded W values into the memory 202. These values are concurrently stored in the memory 202 until data acquisition for the next session begins, ensuring data integrity and auditability.

The integrated system allows for a wide array of advanced bio-informational analyses, including but not limited to Topical qualitative estimation of the functional condition of specific organs or systems. Effectiveness tracking for various forms of treatment or intervention, by comparing pre-and post-treatment signal data. Longitudinal functional analysis, enabling visualization of system dynamics and health trends over time. Localization of functional breaches, identifying the initial nidus or root location of the disorder within the biological system. Expert system evaluations, allowing estimation of the nature and severity of detected changes through algorithmic and heuristic models. Assessment of systemic homeostasis, evaluating the key characteristics that determine the stability and regulatory capacity of the biological system. The telemetric nonlinear analysis data processing apparatus, in combination with the software modules, forms a closed-loop diagnostic platform that not only measures bioresonant properties but also enables interactive feedback for guided therapy. This integration facilitates non-invasive, data-driven, and adaptive physiological evaluation and optimization.

In some embodiments, the at least one processor 200 using the One or more AI models 204 is configured to map the quantified user's quantified emotional load profile and other inferred physiological conditions that are stored in particular organ of the user. The mapping thereby represents how the user feels, relates and move through life. In some embodiments, these relations between the emotional patterns and the organs to help understand the user how the user feels, relates and move through life are stored in the memory 202. Each organ and system within the human body is observed to retain specific emotional imprints that influence physical health, behavioral tendencies, and interpersonal dynamics. According to data stored in the memory 202, liver is associated with the emotional retention of anger, frustration, and resentment. Dysfunction in this organ may manifest through symptoms such as irritability, tension headaches, or muscular stiffness. Energetically, the liver governs the smooth flow of Qi (vital energy), and emotional suppression may lead to stagnation, internal heat, and volatility. Common behavioral expressions include control-seeking tendencies, passive-aggressive responses, and difficulty in articulating personal boundaries.

In some embodiments, a proprietary diagnostic method is utilized to map somatic imprints defined herein as the vibrational or energetic signatures of unresolved quantified emotional load profile and other inferred physiological conditions onto specific organs within the human body. This method employs a frequency-scanning device (the “Biodecodification Scanner”) configured to detect and record the vibrational frequencies emitted by various organ systems. Each organ possesses a characteristic baseline frequency, derived from established scientific literature and empirical research. During the diagnostic process, the at least one processor 200 measures the user's current vibrational frequency for each targeted organ and compares these values against normative baseline frequencies. Deviations from baseline values are interpreted as potential indicators of somatic imprinting—i.e., the physiological manifestation of stored quantified emotional load profile and other inferred physiological conditions within the tissue or energetic field of the organ.

In parallel, a proprietary questionnaire is administered to assess the client's subjective emotional and energetic state. This questionnaire is designed to identify historical and present emotional patterns, cognitive beliefs, and psychosomatic responses. Each emotional frequency category (e.g., fear, grief, anger, shame) is assigned a numerical severity value ranging from 1 to 7, wherein 1 denotes minimal concern and 7 corresponds to a high-intensity, persistent emotional disturbance. The quantified emotional load profile and other inferred physiological conditions is quantified through a combined calculation that includes: (a) the magnitude of deviation from the organ-specific baseline frequencies, (b) the emotional severity scores derived from the questionnaire, and (c) the frequency and consistency of identified patterns across multiple organ systems.

The resulting profile enables the at least one processor 200 to determine the user's total quantified emotional load profile and other inferred physiological conditions, which is then used to classify the client into specific therapeutic categories. These classifications guide the selection of targeted intervention modalities, which may include but are not limited to hypnosis, ThetaHealing, systemic constellations, mediumship coaching, and other specialized therapeutic frameworks. Following the diagnostic phase, a personalized three-month protocol is generated for each client. This protocol includes both emotional coaching and physical detoxification elements, designed to support the release of somatic imprints, reduce quantified emotional load profile and other inferred physiological conditions, and restore the client's system toward an optimal state of homeostasis.

In some embodiments, lungs correspond to the emotions of grief, sadness, and difficulty in emotional release. Physiological manifestations include shallow breathing, fatigue, or chest tightness. As the lungs regulate the body's interface with the external world via respiration, unresolved sorrow or prolonged grief may impair respiratory function and compromise immune response. These imbalances may lead to withdrawal, emotional numbness, and difficulty forming or maintaining intimate connections. Heart is associated with joy, heartbreak, betrayal, and emotional shock, and plays a role in regulating emotional clarity and presence. Disruptions in this region may present as insomnia, palpitations, or restlessness. The heart is the seat of Shen (spirit) in TCM, and emotional wounds particularly related to romantic betrayal or quantified emotional load profile and other inferred physiological conditions may result in excessive emotional reactivity or emotional dullness. Common behavioral effects include difficulty with emotional vulnerability, fear of intimacy, and compensatory people-pleasing behaviour.

In some embodiments, kidneys store emotional imprints related to fear, insecurity, and quantified emotional load profile and other inferred physiological conditions. Dysfunction in the renal-adrenal system may be reflected through exhaustion, hormonal imbalance, low libido, and adrenal burnout. The kidneys are viewed as reservoirs of core life force (Jing), and prolonged exposure to fear especially during early developmental stages may significantly diminish this reserve. Associated behavioral patterns include hypervigilance, attachment insecurity, and reduced trust in life processes. Stomach and gut are linked to emotional states such as worry, anxiety, and overwhelm. Symptoms of imbalance include bloating, indigestion, nausea, and irregular appetite. As the gut plays a critical role in both nutrient assimilation and neurochemical regulation, chronic stress and cognitive overactivity can disrupt digestive efficiency. Behavioral correlates often include overthinking, emotional over-responsibility, and difficulty receiving support.

In some embodiments, pancreas, functionally associated with the spleen in energetic systems, is impacted by emotions such as guilt, diminished self-worth, and a need for over-control. Dysregulation may lead to sugar cravings, energy fluctuations, and metabolic imbalance. The organ's role in managing glucose and energy supply makes it sensitive to beliefs around scarcity and perfectionism. Emotional manifestations include self-neglect, chronic over-functioning, and difficulty accessing pleasure or rest. Gallbladder is observed to retain indecision, resentment, and suppressed anger. This organ aids in directional clarity and supports decision-making. Physical dysfunctions may manifest as tension, digestive disturbances, or migraines. Psychologically, unprocessed truth or avoidance of confrontation may impair clarity and courage. This results in procrastination, emotional withdrawal, and boundary issues.

Muscles and fascia serve as storage sites for unexpressed emotions, unresolved quantified emotional load profile and other inferred physiological conditions, and chronic tension patterns. High-density regions such as the jaw, shoulders, diaphragm, and hips tend to retain protective bracing in response to emotional suppression. Over time, this leads to stiffness, fatigue, and compromised posture. Emotional effects include disconnection from feeling, reduced expressive capacity, and control-based relational strategies. In female pelvic structures, specifically the womb space, emotional storage is associated with shame, sexual quantified emotional load profile and other inferred physiological conditions, creative suppression, and disconnect from feminine identity. Manifestations include pelvic pain, numbness, menstrual irregularities, or reproductive health issues. This area is also linked to emotional intimacy, self-expression, and sensual embodiment. Dysfunction may result in fear of pleasure, emotional rigidity, or lack of creative flow.

In male pelvic structures, particularly the pelvic bowl and root region, emotional patterns center on shame, performance anxiety, and masculine identity conflict. Somatic presentations include lower back tension, erectile dysfunction, and emotional restriction. This region is associated with emotional grounding and embodied confidence. Dysregulation may contribute to emotional avoidance, control issues, and difficulties in vulnerability and deep relational engagement. Through the integration of energetic diagnostics, frequency analysis, and organ-emotion mapping, the system allows practitioners to assess the emotional and energetic landscape of the body. It provides a foundation for interventions aimed at rebalancing both physiological function and emotional expression by targeting the specific storage sites of emotional residues within the body's systems.

In one example embodiments, the liver, which according to psycho-energetic mapping and traditional somatic theory is known to store emotional imprints of anger, frustration, and resentment. In a subject with unresolved quantified emotional load profile and other inferred physiological conditions stored within liver tissue, the at least one processor 200 using the one or more diagnostic sensors 108 applies a targeted external electromagnetic field to stimulate localized disintegration of the meta-stable molecular alignment. As the disintegration occurs, ultra-weak signals related to the organ's functional and emotional state are generated. These signals are typically masked by the background noise of the body's electromagnetic activity; however, by synchronizing with the preset bioelectrical activity of the brain's neurons, the at least one processor 200 amplifies and isolates these specific signal patterns for analysis.

Detection is performed through a non-contact trigger-sensor (not shown) from the one or more diagnostic sensors 108, integrated with advanced microcircuitry. This trigger-sensor is capable of identifying faint fluctuations in the subject's vortex magnetic field and separating them from background statistical noise. Once isolated, the relevant signal is converted by the at least one processor 200 into a digital sequence and transmitted to a microprocessor for real-time processing. The processed data is then interpreted into visual and quantitative indicators of the organ's condition, including emotional and physiological correlations. In one example, where the liver exhibits patterns consistent with Qi stagnation or emotional suppression, the at least one processor 200 may detect disordered electromagnetic resonance in the frequency band associated with the liver's field (e.g., within the 1.8 to 8.2 Hz diagnostic range). The at least one processor 200 then compares this data against known healthy and pathological spectral templates stored in the database. Similar processes may be applied to the lungs (associated with grief), the kidneys (fear), or the gut (anxiety), allowing for broad systemic analysis.

Beyond detection, the at least one processor 200 may further search for and recommend remedies whose electromagnetic spectral signatures most closely counteract the disharmonic oscillations detected thus conceptually “neutralizing” emotional blocks or dysfunctions by applying signals of equal but opposite waveform. This therapeutic application is derived from the understanding that any pathological state can be interpreted as a loss of harmonic synchronization, and correction may be achieved through the application of counterbalancing electromagnetic input. The use of this technology offers the potential to restore functional equilibrium and emotional resilience in a precise, non-invasive manner.

In some embodiments, the biometric analysis system 100 is configured to infer physiological conditions and functional states of one or more organs or biological systems based on analysis of signal characteristics derived from biological data, rather than directly measuring or imaging the organs. The inference is performed using signal processing techniques and one or more artificial intelligence models that identify patterns and relationships between the signal characteristics and physiological states.

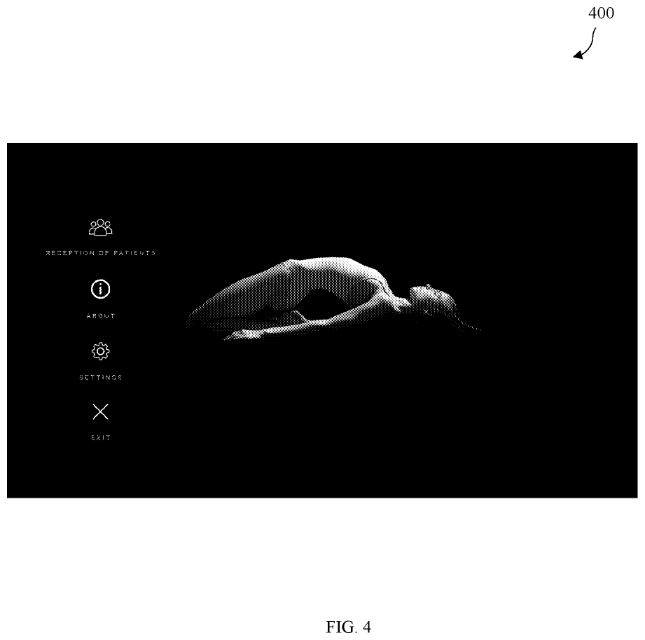

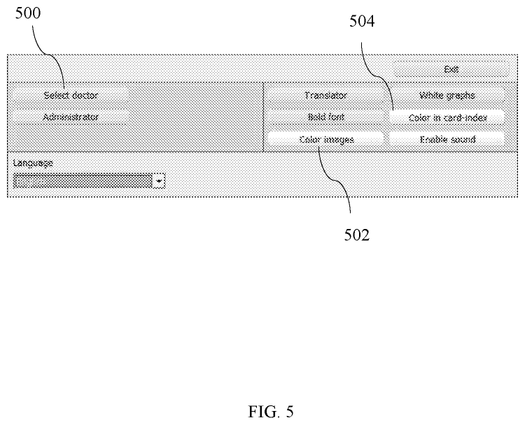

FIG. 4 illustrates the user interface 102, according to an embodiment of the present disclosure. A launching program page 400 may be seen in FIG. 4. In some embodiments, the launching program page 400 of the user interface 102 display a group of buttons names a main menu. The main menu comprises reception of patients, about, settings, and exit. The user may start using the biometric analysis system 100 by clicking the reception of patients'option. Further, as shown in FIG. 5, by clicking the key settings displays the setup form where the user may turn on and off sound, change font size, make the graphs background in white and black, etc. Further, button “Select doctor 500” allows selecting from the list the doctor, who will carry out the researches. Further, the user may turn all pictures in color by pressing “Color images 502” button. Repeated pressing turns pictures to black and white color scheme.

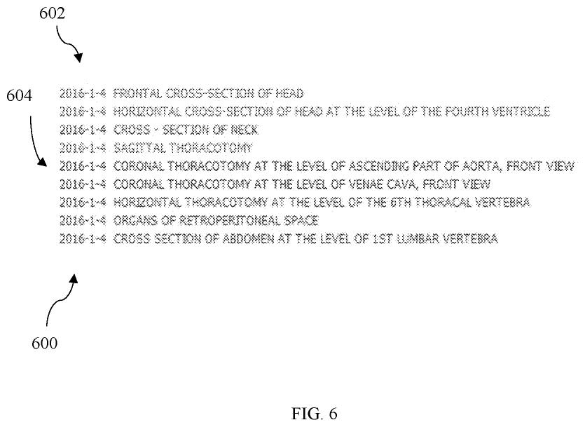

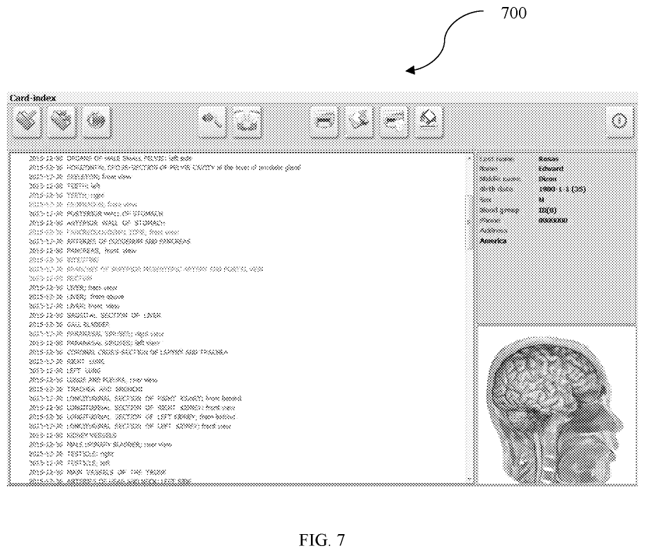

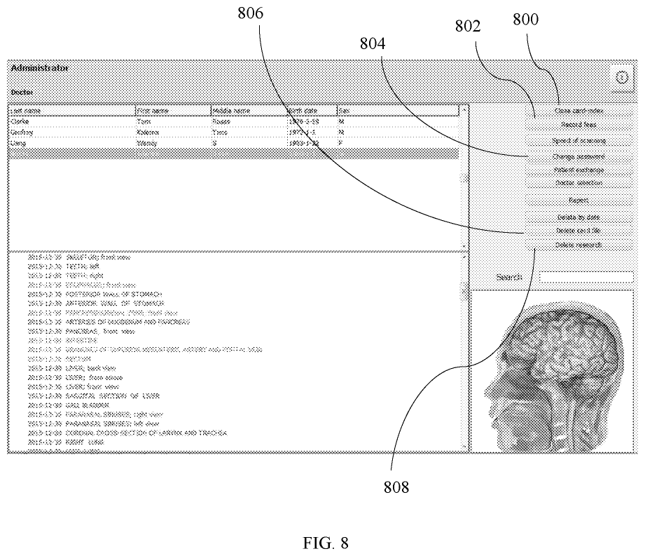

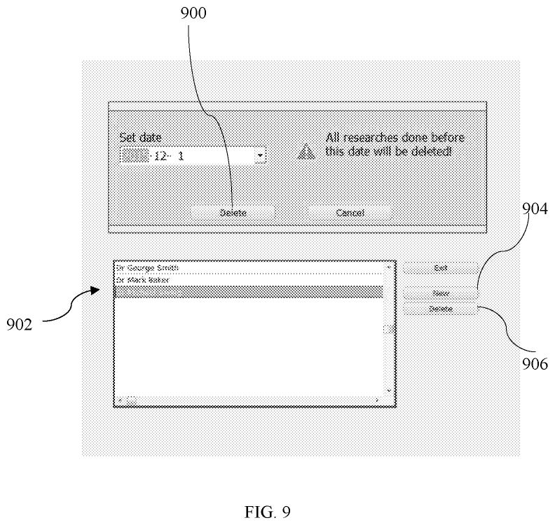

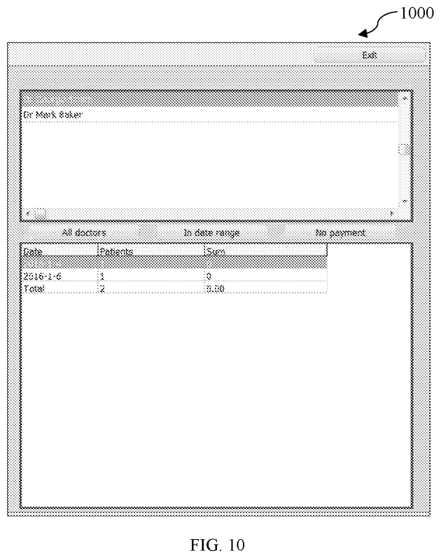

Further, as shown in FIG. 6, Button Color in card-index 504 as shown in FIG. 5 allows the user to highlight organs with different functional stages from already made researches: Green line 600 shows there is no pronounced functional change in evidence. Red line 602 shows minor functional changes. Brown line 604 shows pronounced functional and/or organic changes. Further, as shown in the FIG. 7, before research, the user may make sure that patient i.e. the user is sitting before the apparatus and GR unit, consisting of Bioinductor is on patient's head. Control buttons 700 are shown as pictograms, by pointing on them the user may read a description of them.

Administrator button allows an admin or operator of the biometric analysis system 100 to open or close a card file of patients to other users by pressing “Close card file” button 800. To turn on or off the record fees mode that charges every patient for the completed investigation by pressing “Record Fees” button 802. Access the administration mode by pressing “Change password” button 804, “Delete card file” button 806 in right column removes the card of the selected patient, and “Delete research” button 808 deletes an individual research.

Further, the user may press “Delete by date” button 900 to remove data on all patients before the selected date. In “Delete by date” window user may specify a date; all researches dated earlier that date will be removed. The use may press “Doctor Selection” button 902 to obtain a list of all the doctors using the biometric analysis system 100. Selecting a doctor by name will then display his or her patients. Further, the user may press “New” button 904 to add new doctors-users of the system. Also, the user may remove users by pressing “Delete” button 906, which deletes all patient's records for that particular doctor. Patients may be transferred to another doctor using the same system by pressing “Patient exchange” button.

Further, the user interface 110 may have a “REPORT” button that shows a statistic analysis 1000 of the investigation time, the number of patients seen and the number of investigation sessions made by each doctor or all doctors in the practice. It is also possible to make a financial evaluation of the work carried out in a practice or scientific department. The line “Search” allows to quickly find a patient's card by the first letters of his/her surname. The button “Reception of patients” displays the “Patient's card” containing his/her personal data, such as: surname, name, patronymic, age, sex, blood group, address, phone number. The list of researches takes the most of the form.

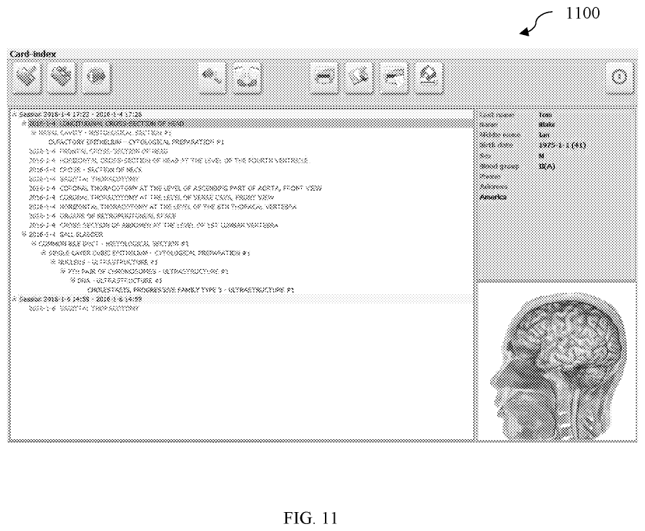

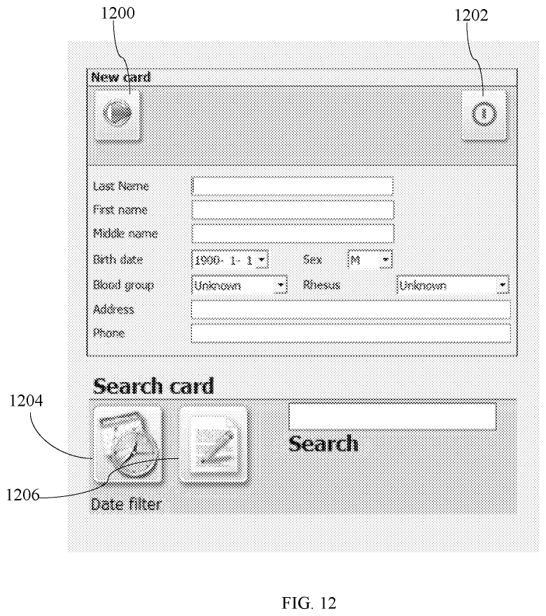

FIGS. 11-12 illustrate a card index feature 1100 of the user interface 110, according to an embodiment of the present disclosure.

In some embodiments, the user interface 110 comprises new card button 1200 allows to input a new patient and says “Please register the Last name*, first name*, sex LOOO Birth date*, sex*, group of bloods* and Rhesus factor*. (as shown in the FIG. 12). Further, a select card button 1202 shows all data about every patient seen by the doctor, namely surname, name, patronymic, age, sex, address and phone number. The user may search for a patient if the user enters patient's surname in “Search” (sometimes it is enough entering few first letters of the patient's surname). If “Date filter” button 1204 is pressed, the user may see dates of researches to the left from patients list. Choosing one of the dates the user may see names of patients that were researched in that day. If user may need to change the information about patients, the user may click “Edit card” 1206.

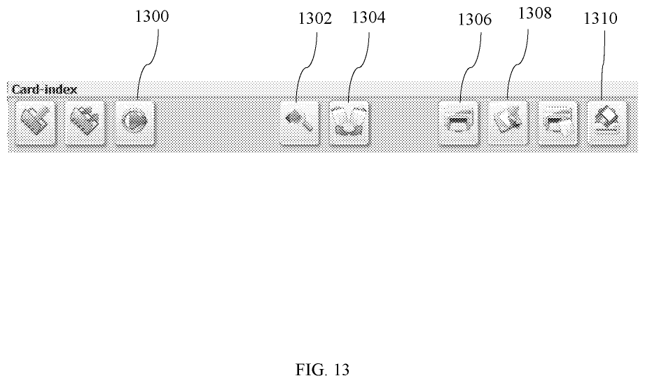

Further, a research button 1300 allows to input a new patient and says “Please register the Last name*, first name*, sex* Birth date*, sex*, group of bloods* and Rhesus factor*. (As shown in the FIG. 13). Further, a current analysis button 1302 is for the analysis of the results taken during the course of investigation, with the possible additional investigations of the patient being made with or without their being connected to the biometric analysis system 100 (the user may press “View the results” button 1304. Further, a comparative analysis button 1306 is a comparative analysis of the results of several investigations with the patient under print researches button 1308 the user may switch to the mode of preparing the researches for printing. The program allows printing out up to 4 pictures sized 100×100 mm on one A4 sheet.

Besides it there is a possibility to look through earlier made “Epicrisis”, opening for analysis and for printing(buttons “Epicrisis” and “Print epicrisis”) the reference processes with graphs taken from the examined patient (at spectral difference factor (D) less than 0.425). The program allows to print out the most effective bioactive supplements picked up by the expert based on the results of the current analysis or to adjust the previously picked up drugs by exiting the form of preparation for printing to the analysis mode (“Analysis” key). Further, a view conclusion button 1310 opens a window in which the physician may write the conclusion about patient's health. The conclusion may be printed out by clicking “Print conclusion” 1312. Further, save to disk 1314 allows writing on a diskette data on researches of the selected patient with the possibility of its further copying into the program.

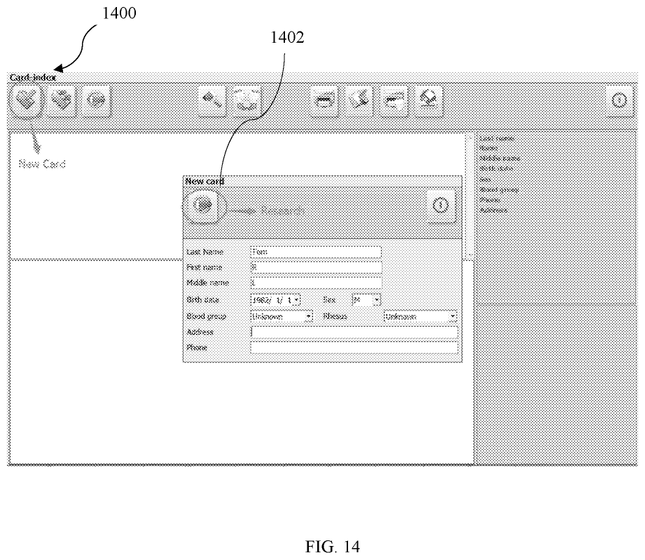

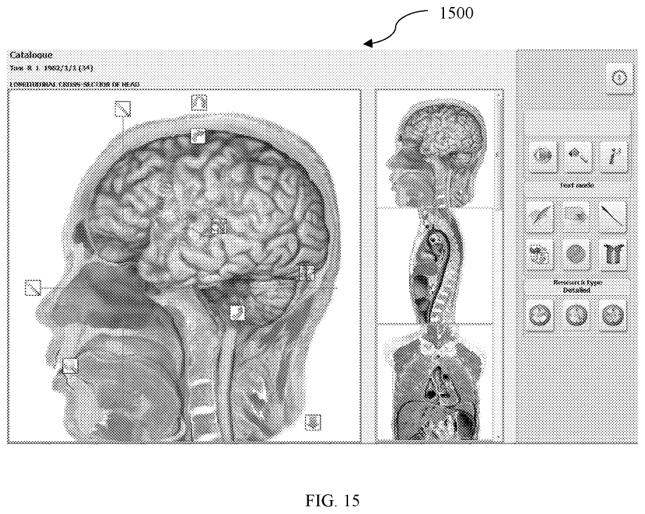

Further in FIG. 14, a new card button 1400 is shown to finish the patient information and click research button 1402. In some embodiments, the button ‘Research’ button 1402 allows to carry out computer nonlinear analysis in the mode of a programmed and (or) individual selection of the organs intended for researches. The main feature of the software is “scan” function 1500 as shown in the FIG. 15, which allows automatically localize the nidus where tumour and hereditary diseases appeared, find out the reason of appearance on genome level, passing one by one histological, cytological preparations, chromosomes and going deeper to the level of DNA molecule parts.

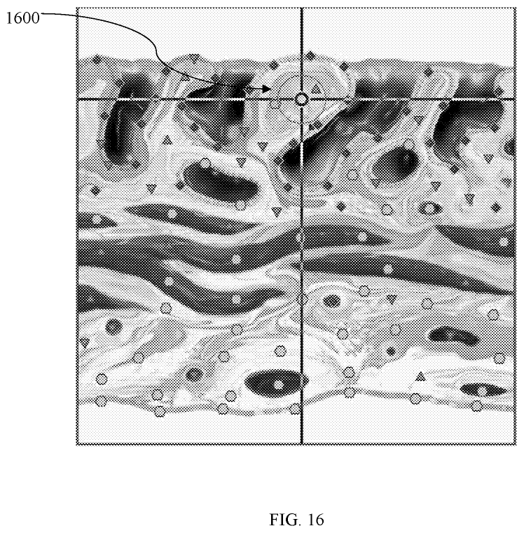

Further, as shown in FIG. 16, the user may turn a catalogue mode, where on this mode by pressing “scan” button 1600. If this button is not pressed, researches will be carried out in normal mode, without localizing of nidus where pathology appeared. During the research, the changes in tissues shown on macrocuts are revealed, then search and research of histological cuts of these tissues in areas of the most significant pathological changes are carried out. Then, after histological cut research, the search for the most significantly changed cells is carried out, which examined to reveal changes in cell structures. After that, the algorithm of the search goes to the level of chromosomes, to estimate the changes in single chromosomes, then it goes deeper to the level of DNA molecule research. During the research in “3D scan” mode the estimation of topological picture and metastasis is carried out. Additional researches fulfilled to search metastases into other organs. The research is done in automatic mode; user may observe it and stop at any moment.

Further, an “ectomies” button 1700 allows to preclude an attempt to examine patient's resected organs. Further, a “switch to manula/auto choice” button allows an automatic selection of a profound detailed researches of anatomical, histological and cytological structures depending upon the presence of frank changes in complete anatomical sections of the body; when the button is not pressed the physician may solely select for researches the organs belonging to one of the anatomical systems, by putting or removing tags on the organ's picture, in the right part of the screen on the split bar with right mouse button. Further, using a “remedy preparation” button 1702 the preparation is made automatically for pictures, in which the nidi were evaluated. After clicking this button, the operator should select the organs subject to preparation making and press a “Start preparation making” button.

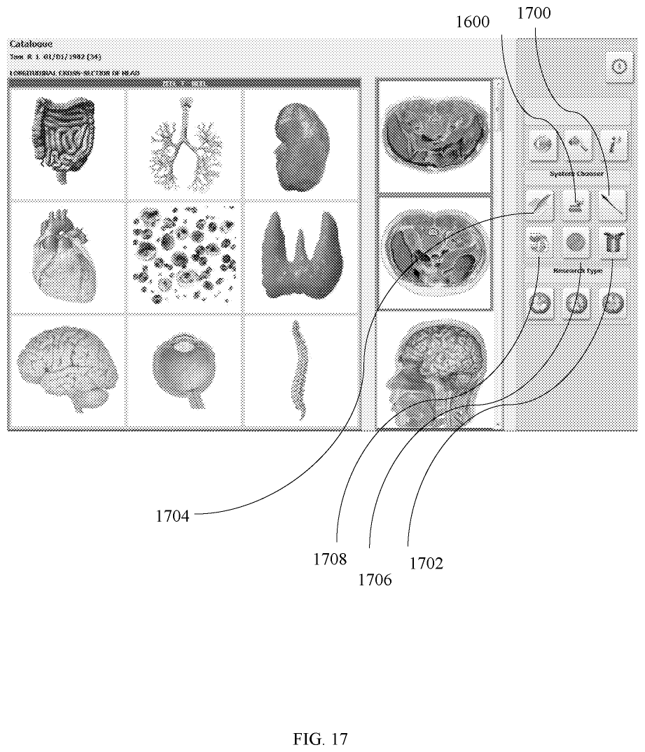

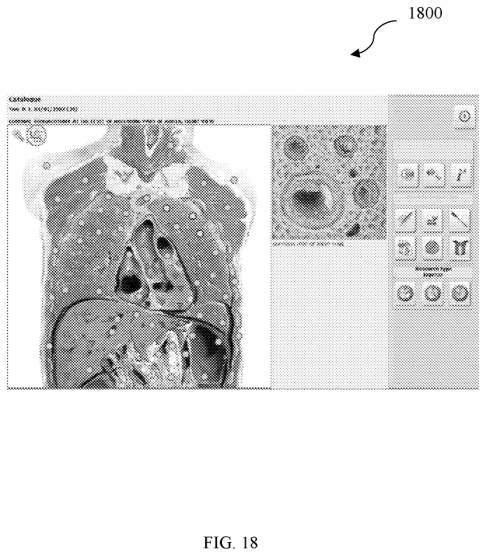

In organs catalogue there are two modes of representation: Graphical—organs as shown in the FIG. 17; Textual—shown names of organs. User may switch modes by pressing “Text mode” button 1704. In text mode “Sort” and “Cancel/Restore chosen” buttons are available. “Sort” button allows change modes of organs list sorting. There are four modes i.e. are “According to Systems”: Organs sorted according to systems: main catalogue, digestive system, respiratory system, urogenital system, cardiovascular system, blood and lymph, endocrine system, nervous system, sensors and musculoskeletal system. “According to Alphabetical order” mode, “According to Isolines” mode that positions of isolines are compared. Organs with higher positioned S-graph shown in the beginning of theist. “According to Points” mode: in the beginning of the line shown organs with more pronounced changes in estimation points. “ACCUPUNCTURE” button 1706 allows entering the bioactive points research mode. The extended view pane now contains accessible images of organs (hands, feet, auricle, iris) intended for evaluation of the acupuncture points.

Clicking a “Select system” button 1708 displays 9 buttons with images of the organs typical for respective anatomical systems. After selecting a particular system, only organs of this system become accessible on the extended view pane, and the upper line will contain its name. To return to the basic catalogue organs the operator has to repeatedly press the button corresponding to the selected system. An “additional projection/ultra-structure switch” button allows engaging the modes “Additional projection/ultrastructure”. In “Additional projection” mode, the organ's image contains icons with which the user can quickly switch between organs. Pointing the cursor at the icon to the right of the control buttons displays the organ's image and name. Clicking the icon unfolds this organ. To return to an initial organ a button located in the upper left corner of the image is pressed.

Further, in a ultra-structure mode as shown in the FIG. 18, histological, cytological and molecular researches are carried out. Moving the cursor over organ's projection, the user may select the structure of user's interest, which image and name appear on the right, and the cursor becomes a cross-hair. Clicking the left mouse button starts researches of a respective structure at the preset point. After the researches are completed, an icon emerges on the organ's image, which may be unfolded by clicking it with the left mouse button, as shown by 1800. On one and the same organ user may make several researches of ultra-structures with different localizations, gradually passing from microscopic sections to cytological preparation and molecular structures.

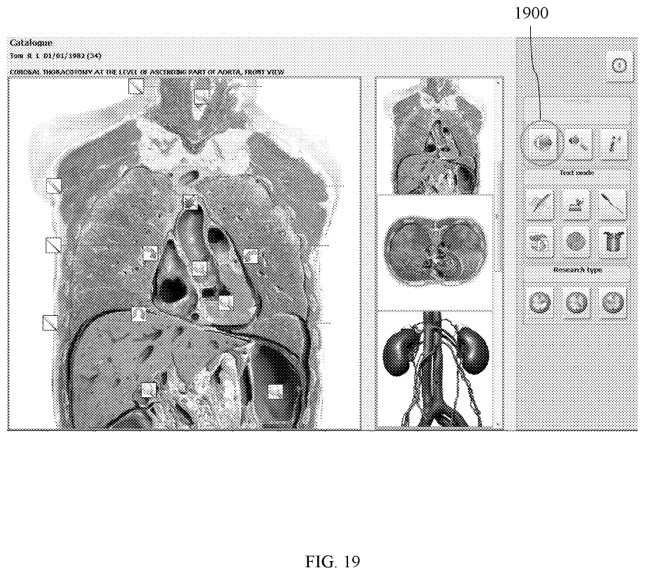

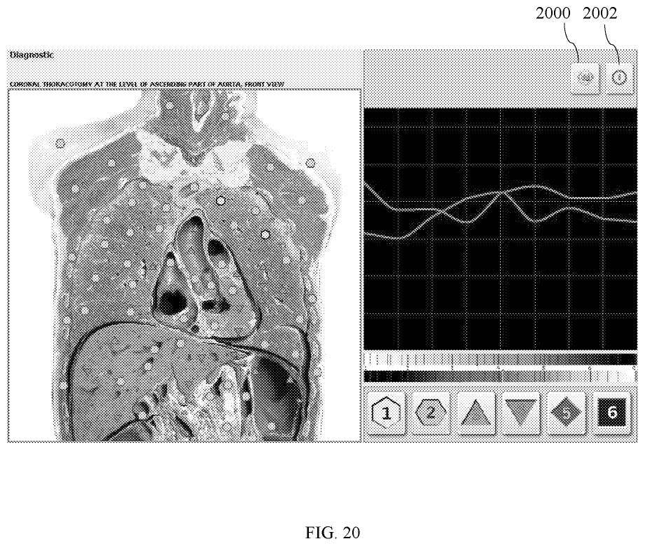

Types of researches comprises at least “Express” that enables investigations to be carried out on the full topographic sections without details. “Standard” enables the study of individual histological or genetic structures to be carried out, provided they show pathological changes. “Detail” enables an evaluation to be made of the structure of all body tissues on histological, cytological or genetic levels, which may be important to high quality scientific research. Further, as shown in FIG. 19, by clicking “research” button 1900 may will start scanning. Further, as shown in FIG. 20, a “Pause/Resume” button 2000 interrupts the research. When pressed the button changes to “Resume” and when released the research will continue automatically. The “Next/Exit” button 2002 make the next research or exit the research.

Further, the user interface 110 comprises more icons corresponding to a section, matching tissue or cell target, even body subject to another target (where the direction of the arrow indicates the direction), associated body (where the direction of the arrow indicates the direction) symmetric target organ. Matches a more detailed search of the area corresponds to a search of the different organ or system but related area. Nervous system related indicates respectively: Bloodstream, Arterial, Venous, Circulatory System. Arteries and veins and Zoomed in/out the pictures of organs. In the “Researches” mode the user may be able to measure the frankness of the destructive process at reference points standardly placed on the picture, the results being evaluated on six point polychrome Fleinder's scale which buttons are located in the lower right corner of the screen.

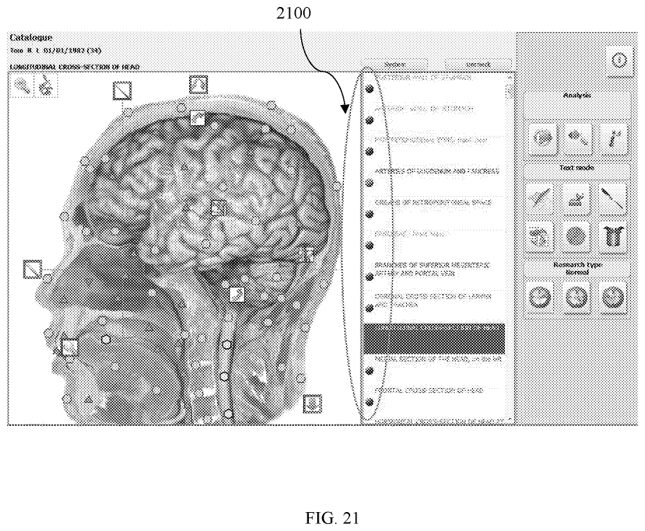

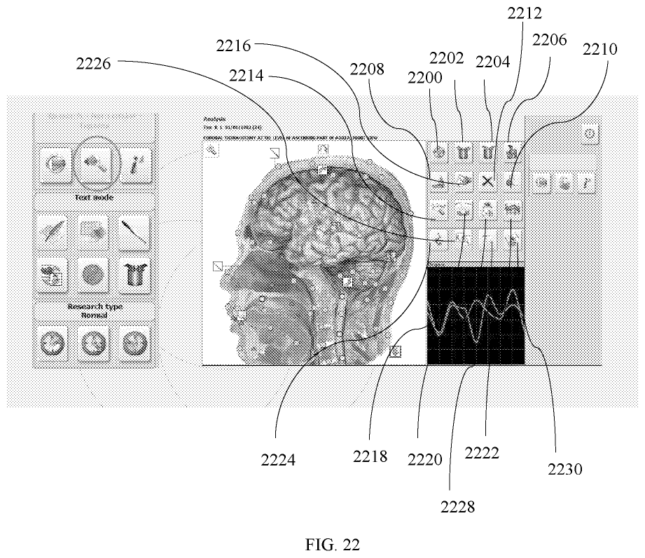

As shown in FIG. 21, upon finishing the research, the user interface 110 may show an investigation list with red, green and brown dots by 2100. The green dots mean that the organs are generally good, the red dots mean the organs are slightly dysfunction and the brown dots mean the organ beneath the subordinated part. Further, upon clicking on “Analysis” button the user interface 110 may show a pages as shown in the FIG. 22. The page may have multiple icons that comprises “META-THERAPY” button 2200 that makes possible to affect the organism correcting under the use of the emitter. It is necessary to furnish the centre of the effect on the range of the largest information loss. “VEGETO TEST” button 2202 offers two possibilities: internal and outside vegeto test. The internal vegeto test LLLL compares, from the database of the program Metatron, selected frequency characteristics of e.g. homeopathic means or food additions mathematically with body-own oscillations of the test person.

The exteriors vegeto test is accomplished from the mode analysis. The too examine preparation is placed in the resonance chamber, which Button vegete test activates. The designation of the preparation is registered into the opening window. “REPRINTER” button 2204 permits to note each frequency characteristic from the data base of the program LLLLL on the matrix. From the mode catalogue of the comparison samples. If the preparation is selected (D “may not more highly than 0.425), then the key Reprinter is to be clicked. “MAKE METAZODEN” button 2206 is analogous to “Reprinter” function, but inverting and additional processing of the signal carried out in automatic mode from selected pathological nidus. “MORE PRECISE NIDU” button 2208 allows either to hide or to show the previously selected nidi on the organ's picture.

Further, “CONTOUR ON NIDU” button 2210 makes possible to affect the organism correcting under the use of the emitter. It is necessary to furnish the enter of the effect on the range of the largest information loss. “DELETE CONTOUR ON NIDU” button 2212 offers two possibilities: internal and outside vegeto test. The internal vegeto test LLLL compares, from the database of the program Metatron, selected frequency characteristics of e.g. homeopathic means or food additions mathematically with body-own oscillations of the test person. The exteriors vegeto test is accomplished from the mode analysis. The too examine preparation is placed in the resonance chamber, which Button vegeto test activates. The designation of the preparation is registered into the opening window.

“IMAGES IN BW/COLOR” button 2214 permits to note each frequency characteristic from the data base of the program LLLLL on the matrix. From the mode catalogue of the comparison samples. If the preparation is selected (D “may not more highly than 0.425), then the key Reprinter is to be clicked. “TEST STANDARD” button 2216 is analogous to “Reprinter” function, but inverting and additional processing of the signal carried out in automatic mode from selected pathological nidus. “COMPARATIVE ANALYSIS” button 2218 allows either to hide or to show the previously selected nidi on the organ's picture. “RESULT OF INFLUENCE” button 2220 may analyze results of “Vegeto-test” and “Meta-therapy” carried out for chosen organ. When the user pressed this button, the user has to choose the research, which the user wants to open.

Further, “SPECTRUM” button 2222 displays an enlarged graph with values of signal amplitudes by spectrum frequencies. “ULTRASTRUCTURE” button 2224 shows histological, molecular and bacterial researches. The user may unfold the ultrastructural research by clicking a respective icon. “PICTOGRAMS” button 2226 activate or deactivate the pictograms. “DESCRIPTION STRUCTURE” button 2228 may indicate the description of the structures. By clicking the green cross, the appropriate designation appears on the screen. “DESCRIPTION FUNCTION” button 2230 shows the description of a biological object represented on the picture.

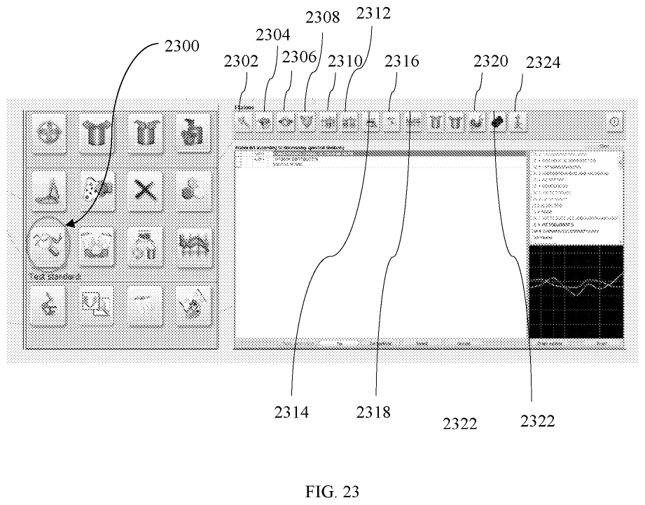

Further, as shown in FIG. 23 by clicking a “ETALONS” button 2300, etalons are shown. “SMART FILTER” button 2302 allows the user to sort processes at the same time after selected headlines as well as biggest spectral similarities. “VIRTUAL MODEL” button 2304 places the addition of the Etalons, which the program selects from the data base as probable diagnosis. “ENTROPY ANALYSIS” button 2306 permits to accomplish a two-factor entropy analysis of the pathological process. The entropy analysis permits to develop the mathematical model of the dynamics of the pathological process. “NLS-ANALYSIS” button 2308 permits to accomplish a multi-factor entropy analysis of the pathological process (e.g. the biochemical homeostasis of the system).

The evaluation of biochemical homeostasis takes place in the mode of the NLS analysis. “ADJUSTMENT” button 2310 causes tuning of the chosen reference process to the spectral characteristics of the object under investigation and shows the process in its activation phase. “ADJUST ALL” button 2312 causes the tuning of ALL reference processes stored in the etalons directory referred to under Adjustment. This is the button necessary to do prior to view and interpretation (Entropy Analysis) of current disease states. “TO EPICRISIS” button 2314 prints an etalons with a high spectral similarity to the researched object. The user may send to epicrisis several etalons at once marking them to the left of their names.

“VIEW EPICRISIS” button 2316 displays on the screen the list of etalons chosen for printing. “ETALON-OBJECT” button 2318 carries out a dispersion analysis with reference to an etalon chosen at random with the object functions assigned to it. “BACTERIAL ANALYSIS” button 2320 is accessible only if dispersion of the etalon from ‘Microorganisms’ is less tLLLL than 0.750, which may suggest presence of non-activity of this microorganism in biological tissues) starts the bacterial analysis process. “LITHOTHERAPY” button 2322 starts a mode of treatment with use of spectral characteristics of chosen mineral. Control elements are the same as in “Meta-therapy” mode. “PHYTOTHERAPY” button 2324 starts a mode of treatment with use of spectral characteristics of chosen mineral. Control elements are the same as in “Meta-therapy” mode.

Further, “ETALON DESCRIPTION” button displays the detailed description of the selected etalon. FIX Allows fixing these three lines in the top part of the list: Biological object name. Optimal distribution. Virtual model. “SORT OPTIONS” button opens a drop-down list, which allows selecting the modes of etalons arrangement by spectral similarity, alphabetically, by potential activity (rate of change of the etalon dispersion (d) in the forecasting mode). “SELECT” button allows selecting a group of etalons similar to each other by the last word in their name. “GROUPS” button allows defining the number of etalon groups different by last word of their names, in one system. In the ‘Groups’ mode the user may select several etalon groups (with the right mouse button) which will be returned after clicking the ‘select’ button.

Further, “GRAPH OPTIONS” button provides a list with options of the illustration of the chart. “SPECTRUM” button displays an enlarged graph. “OPTIMUM” button of yellow graph and shows normal (Gaussian) distribution of the signal in standard frequency. “OBJECT” button of Graph lines of lilac and orange color, display a graph of the examined biological object, organ or tissue plotted in the course of the investigation. “ETALON” button of thin graph lines of red and blue colors, represent a graph of the chosen reference process. “MODEL” button of thick graph lines of red and blue colors, show a graph of the virtual model. “INVERT” button enables the polarity of the graph to be inverted. “CLEAR” button above the list of etalons, removes the dispersion analysis from all groups of etalons.

In some embodiments, every organ and every cell have its own specific and distinctive oscillation. The oscillations are stored in the memory 202 and may be displayed on the screen i.e. the user interface 110 as a graph, which represents the condition of the information exchange between an organ or tissue and the environment. Every pathology has its own individual and distinctive graph. The memory 202 also stores a very large number of pathological processes taking into account rate of evidence, age, sex and other variations. After frequency characteristics are read from tissue, the apparatus compares spectral similarity with stored processes (healthy tissue, pathological tissue, infection agents) and then selects the closest pathological process or tendencies to its appearance. By this method of analysis, it is possible to trace the condition of the red (S) input signal and the condition of the blue (N) output signal, which are displayed as graphs on the screen. From the shape of the graph, it is possible to determine which of the reference (etalon) processes it appears to be closest to and to check the spectral similarity of the graph plotted for the patient.

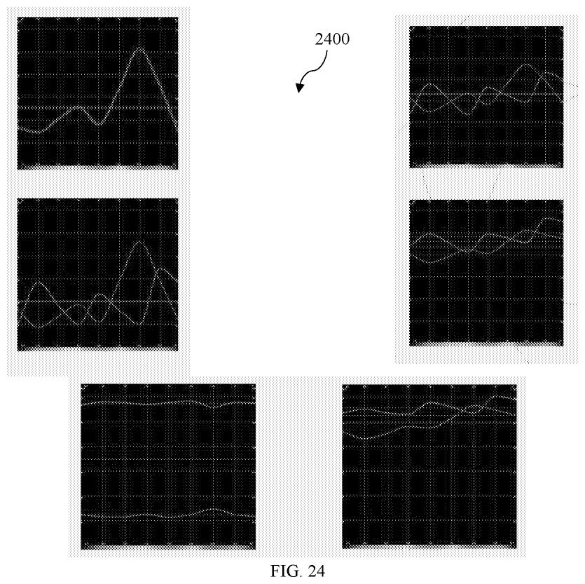

As shown in the FIG. 24, the apparatus functions on the principle of evaluating the noise rate within a biological system, conceptualized as a cybernetic “black box” 2400 where organs, tissues, or even individual cells are treated as systems with measurable input and output signals. A control signal (denoted as S, shown in red) is introduced into the system, and the resulting response signal (N, shown in blue) is measured and analyzed. By comparing these signals, the system assesses the entropic potential, which reflects the system's functional integrity. In a healthy state, input and output signals are similar, indicating low internal noise and optimal operation; conversely, a lack of response suggests non-functionality.

Practical readings typically fall between intermediate values and are represented using a graph amplitude scale where 0 dB equals 0 points and 260 dB equals 6.6 points, with healthy tissue showing natural noise levels between 8.5 to 64 dB (1 to 3 points). The functional evaluation range is between 1 and 6.5 points, while values beyond this indicate system failure. Self-resonant frequencies are also analyzed, with higher-order tissues (e.g., brain cortex) exhibiting frequencies up to 8.2 Hz, and simpler structures (e.g., bone) resonating around 1.8 Hz. Importantly, the S and N graphs are interpreted together rather than independently, with attention given to frequency bands exhibiting the most dissociation and the graph showing greater amplitude, as it reflects priority in the pathological process. Isolines are overlaid to aid analysis by highlighting critical areas and revealing the acuteness and progression of physiological changes, thereby guiding subsequent diagnostic and therapeutic decisions.

The diagnostic system further incorporates a “Spectrum” function, which provides a visual graph of the physiological process by displaying three isolines: a central yellow isoline representing the simple average of both signals (input and output), a red isoline corresponding to the input signal (S), and a blue isoline corresponding to the output signal (N). This graphical output enables precise analysis of compensatory dynamics within the biological system. The interval between the N and S isolines is a critical metric: if the interval is less than 0.25 points, the process is classified as compensated, indicating that the system's internal reserves are sufficient to regulate and counteract the pathological influence independently. In compensated states, tissue and systemic reserves operate synchronously, representing the most favourable condition for therapeutic management. If the interval exceeds 0.25 points, the process is considered decompensated and can manifest in two distinct forms anabolic and catabolic.

An anabolic decompensated state is typically indicated when the blue isoline (output) is higher than the red (input), reflecting a process of energy and information discharge, and is associated with acute clinical presentations such as phlegmon, abscesses, or pneumonia, often accompanied by autoimmune or hyperergic tissue responses. While this phase may initially reflect active compensatory attempts, tissue reserves are rapidly depleted, leading to a crisis state unless recovery mechanisms are engaged. If tissue-level compensation fails, the condition progresses into a catabolic process, identified by the red isoline (input) being positioned higher than the blue (output), reflecting the organism's absorption of information and energy. Early catabolic stages may be subtle and associated with chronic conditions, particularly when the middle yellow isoline reads at or above 3.5 points. With disease progression, the middle isoline may elevate to 5.5 points or higher, while graph voltage defined as the amplitude interval between graph peaks and their isoline begins to diminish. This decline in voltage is indicative of reduced functional response and systemic control, marking a more severe degeneration of physiological homeostasis.

In further analyzing signal behavior using the system's spectrum function and isoline interpretation, the at least one processor 200 is configured to identify patterns in tissues that demonstrate reduced or altered responsiveness to an input control signal. In some embodiments, the interval between a first isoline corresponding to an input signal and a second isoline corresponding to an output signal may increase, indicating reduced signal responsiveness and increased energy expenditure associated with maintaining physiological function.

In some embodiments, variations in isoline spacing, amplitude characteristics, and resonance behavior may correspond to differing levels of physiological or functional deviation within one or more tissues or biological systems. For example, a moderate increase in isoline interval may correspond to partially reduced responsiveness, while a larger increase may correspond to significantly reduced or disrupted signal response. Similarly, changes in signal amplitude and resonance characteristics across frequencies may indicate localized or systemic alterations in functional behavior of the biological system. In some embodiments, such signal patterns are used by the at least one processor 200 to characterize system-level imbalances and functional deviations without identifying or diagnosing specific medical conditions.

In some embodiments, the at least one processor 200 is further configured to evaluate a degree of signal variation based on analysis of relationships between multiple signal representations, including one or more signal graphs and corresponding isolines. The evaluation may include determining a degree of divergence between the signal graphs and analyzing positional variations of one or more reference isolines. In some embodiments, a first level of functional deviation is characterized by localized divergence between signal representations at one or more frequencies, with corresponding isoline values within a defined range. In other embodiments, a higher level of functional deviation is characterized by divergence across multiple frequencies and elevated isoline values, indicating broader disruption in signal behavior across the biological system. In some embodiments, the at least one processor 200 utilizes nonlinear analysis techniques to classify levels of functional deviation and system-level imbalance based on the observed signal patterns. The classification may support identification of relative severity and progression of functional states without diagnosing specific medical conditions.

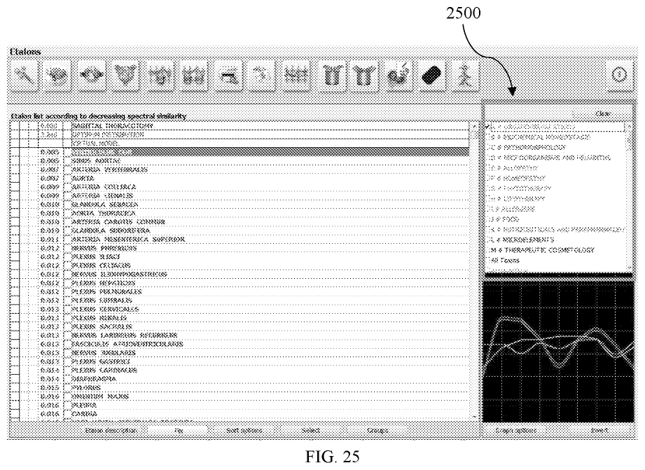

Further, as shown in FIG. 25 “Group List” 2500 contains a list of etalon groups. The list has a plurality of feature comprising organ operations, biochemical homeostasis, patamorphology, microorganisms and helminths, allopathy, homeopathy, phytotherapy, nutriceuticals and parapharmaceuticals, allergens, food, and lithotherapy. The organ operations contain the etalons (standards) of the spectral characteristics of healthy body tissues. The etalon list is arranged in a decreasing order of spectral similarity to the object under investigation. Healthy tissue has similar input and output signals (the red and blue graphs respectively). The greater the similarity between the organ preparation and the object under investigation, the more intact the tissue will be. Conversely, the greater the difference the greater the damage will be to the tissue under investigation.

The biochemical homeostasis carries out a qualitative evaluation of the main biochemical factors by evaluating the wave functions of body tissues. This evaluation is carried out using the NLS-analysis mode. Note that the lowest values of enzyme (hormone) concentration within the normal bounds correspond to 2 in the graph. Whereas, the highest values within the normal bounds correspond to 6. The values of the factors equal to 3, 4 or 5 correspond to the ‘mode’ of the factor, and the extreme values 1 and 7 characterize biochemical factors beyond the physiological norms, lower and higher respectively. The standard rules for making biochemical analysis using conventional clinical methods should be used when analyzing the computer results.

Further, the patamorphology feature shows a list of the etalons of destructive processes. this section holds basic patomorphologic conditions peculiar to single tissues of an organism. every destructive process has a distinctive graph. In the microorganisms and helminths in this section are recorded the major characteristics of infection agents:—bacteria, viruses, mycoplasma, rickettsias, fungi, and helminths. This presents changes in the form of high peaks of dissociation within the frequency range representing the natural frequencies of the tissue. Allopathy it is the wave characteristics of the principal chemical (synthetic) medicinal preparations used in conventional medicine. Homeopathy, in this group the wave characteristics of homeopathic preparations are recorded.

Further, “phytotherapy” in this group-the wave characteristics of medicinal plants are recorded. Further, “nutriceuticals and parapharmaceuticals” in this group-the wave characteristics of biologically active supplements (bas) produced by various companies supply nutritional supplements are recorded. “Allergens” in this group—the wave characteristics of food, domestic, animal, vegetable, and industrial allergens of all kinds are recorded. “Food” in this group—the wave characteristics of food separated into groups (meat, fish, dairy, vegetables, fruits, oil, drinks, spices, etc.) are recorded. Dietetic foodstuff, due to their therapeutic action recommended to use, highlighted in green color. Not recommended foodstuff highlighted in black, neutral—in orange. “Lithotherapy” in the program spectral characteristics of gems and minerals are represented together with description of therapeutic action. By pressing “picture” button, the user may see pictures of more than 200 minerals. A program can choose a mineral for a patient, suitable according to spectral characteristics for permanent possession or for specific diseases treatment. Using specific spectral characteristics of minerals, modulated and radiated by the apparatus, the user may treat acute and chronic diseases.

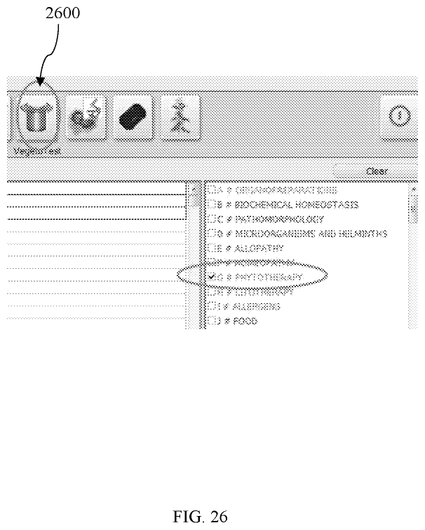

Further, as shown in FIG. 26 shows “Vegeto test” 2600 that allows to simulate the process of influence of a chosen etalon to an organ. For this purpose, we choose an etalon and carry out the Vegeto test (Bio-inductor (headphones) should be on client head). Choose the etalon and click the button Vegeto-test situated on the right, the Vegeto-test will begin automatically.

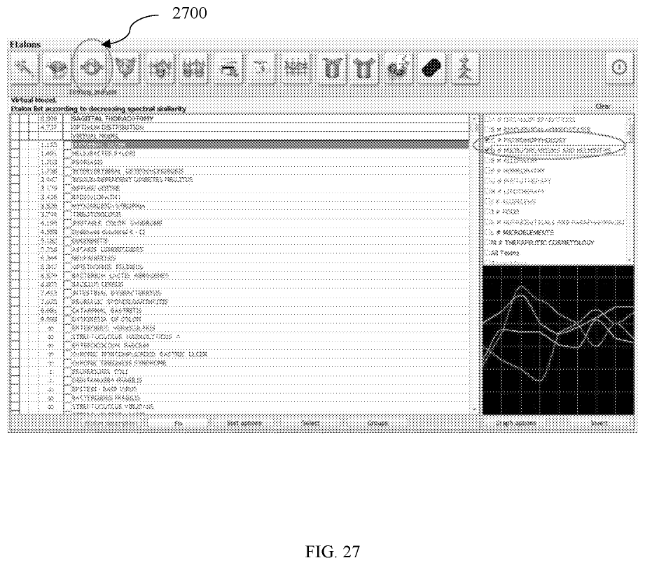

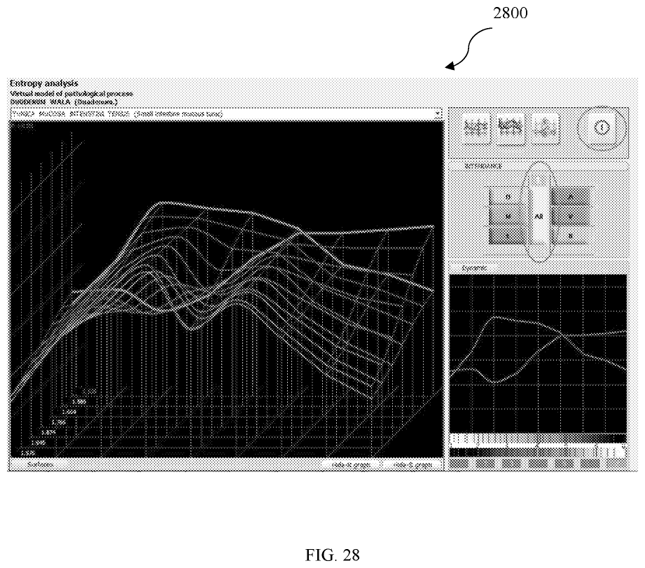

FIG. 27 shows an “Entropy analysis” button 2700. The biometric analysis system 100 additionally employs an entropy-based two-factor analysis module designed to construct a mathematical model of pathological development by defining a continuum between two biological extremes: a healthy tissue state, considered the initial (zero) phase, and a clinically manifested pathological condition, representing the final state. This entropy model is computationally derived using nonlinear spectral data and allows the generation of graphs corresponding to a series of intermediate conditions. During analysis, the system calculates the degree of spectral similarity between the biological signal under evaluation and each of these intermediate or extreme reference states. This process enables the identification of the maturity level of the pathological condition and facilitates early detection of preclinical abnormalities. A key diagnostic marker within this model is the dispersion of entropy factors across the defined range of values, typically from 1.0 to 6.0.

A low dispersion value (i.e., minimal variation across this range) is indicative of an acute pathological process, signaling a more uniform progression toward a defined pathological state. The system highlights the entropy factor with the greatest spectral similarity using a bolded line within the graph, while the corresponding numeric value is displayed in red text—for example, a reading of 1.435 may be shown to denote the best spectral correlation. The interface includes functional options such as “Conceal N (S) graph” which allow the user to isolate and view either the input or output signal independently, aiding in advanced interpretive analysis. Additionally, the graph visualization can be toggled between a “Lines” mode, which displays standard isolines, and a “Surfaces” mode for enhanced topographic representation of the data, thereby providing the practitioner with multiple perspectives for evaluating systemic entropic changes 2800 (as shown in FIG. 28).

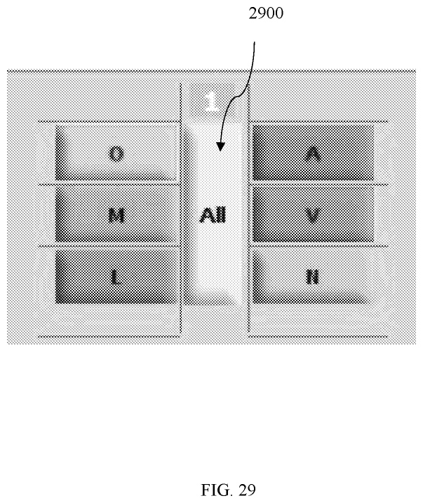

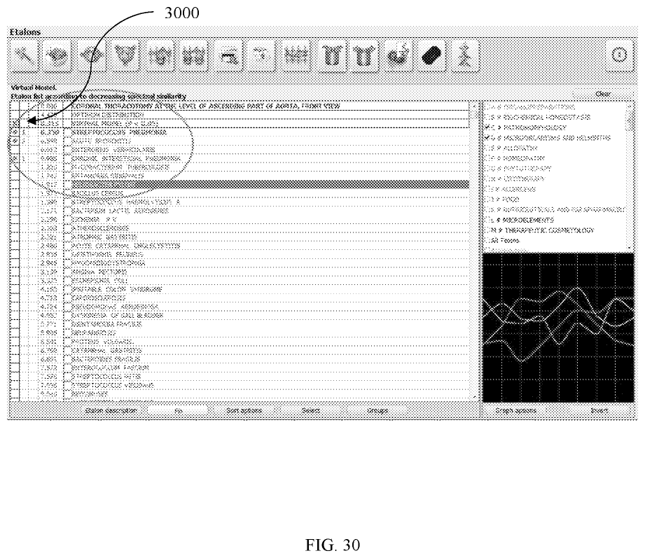

In some embodiments, “Intendance-screening” determines the extent of deterioration of different tissues in a developing pathological process. It is possible to trace these pronounced pathological changes in all groups of tissue structures simultaneously (by pressing an “ALL” button 2900) or in individual morphological groups. Further, the user interface shown in FIG. 29 comprises ‘O’ (lilac) shows the remaining groups of tissues not included in the above. Object displays the optimum values for this process. Vera-Test represents a graph with the closest spectral similarity. Dynamics—It is possible to trace the dynamics of the process. Spectrum 2910 displays an enlarged graph. These results will be shown in the FIG. 30. As shown in the FIG. 2900, “M” represents muscular system, “L” represents lymphatic system, “A” artery system, “V” represent Vien system and “N” represents nervous system.

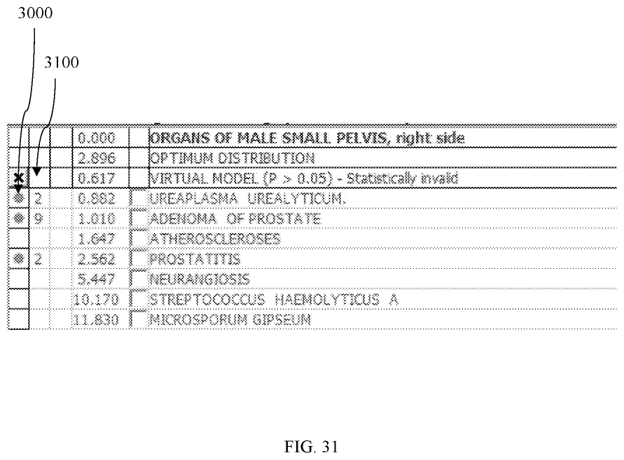

Further, as shown in FIGS. 30 and 31, dot 3000 may be seen that represents the disease have occurred in this state and also have potential warning. If the dot on the top or upper indicates the disease is developing in this state. Further, numbers in arrow 3100, shows the time of the disease happening. The first level (1~3): Time is short or have occurred in the recently. The second level (4~6): Time is in the past six months or time of occurrence for too long. The third level (7~9): Time is one year or more than one year. The fourth level (∞): Have happened for a long time or long-term. Further, as shown in FIG. 31 shows D value in fourth column in red. If <0.425, the spectral similarity to the object under investigation is over 95% with the etalon being marked red. If 0.425~2.0, It may not show symptoms now, if the user pay no attention, then the problem maybe will happen soon (chronic). If >2.0, Means lack of any matching samples, the morbidity is very small.

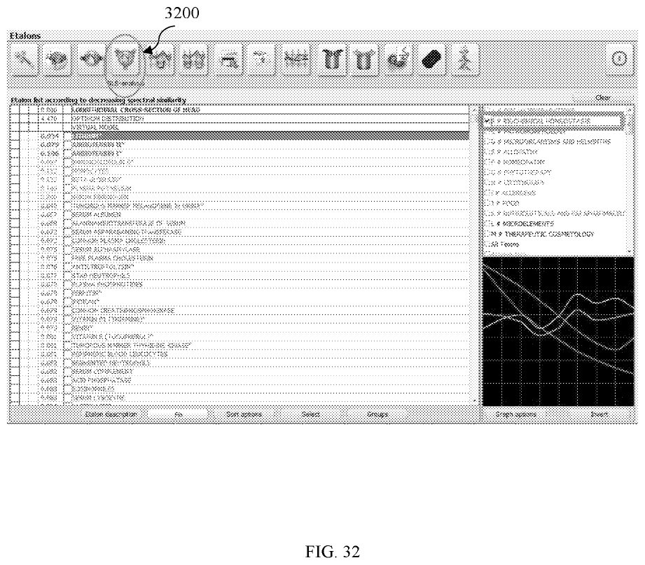

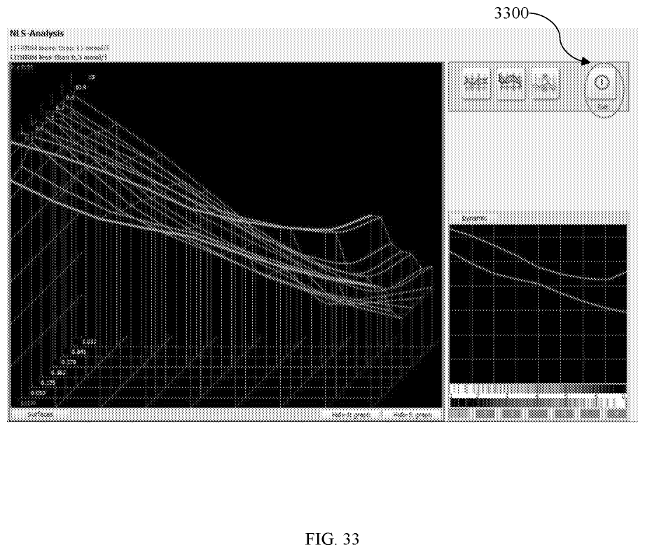

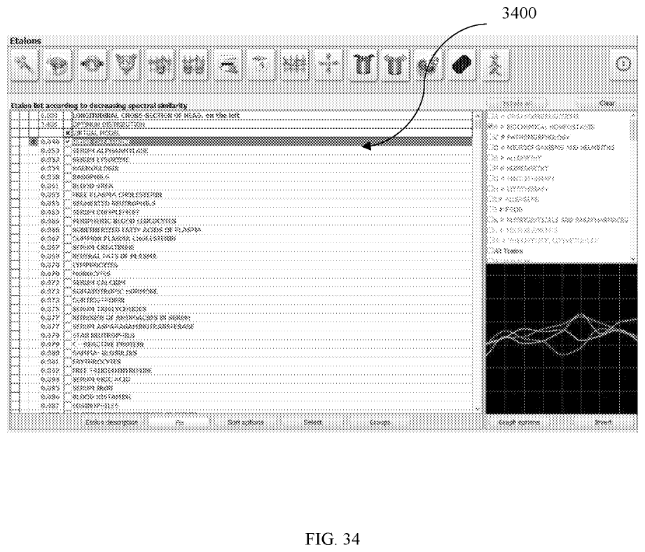

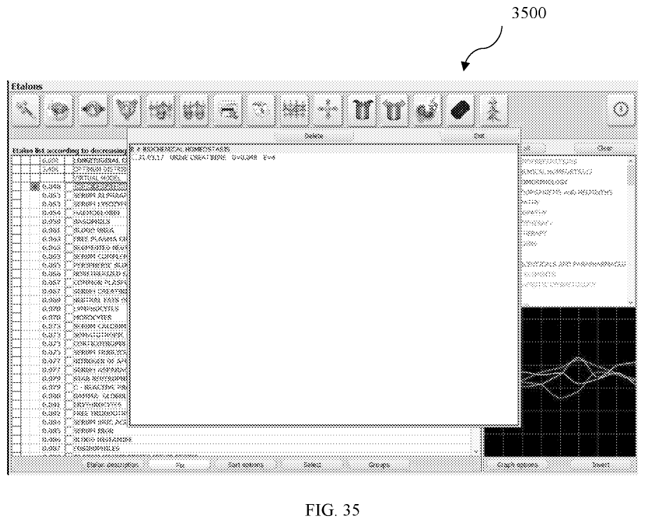

Further, FIG. 32 shows a NL analysis button 3200 that may be clicked by the user. Further, the user will see a picture where an exit button 3300 may be clicked as shown in FIG. 33, upon which as shown in FIG. 34, results may be shown by 3400. In third column E value is the same as the Report E value as the following. Analysis of the D value<0.425:1, indicates the biochemical index is lower than the standard value range. 2~6, indicates the biochemical index in a safe range. 7, indicates the biochemical index is higher than the standard value range. If NLS-analysis extreme values 1 and 7 characterize biochemical factors beyond the physiological norms, lower and higher respectively. Further, the user may do entropy analysis and may click “To epicrisis” button and “View epicrisis” button shown by 3500, as shown in FIG. 35.

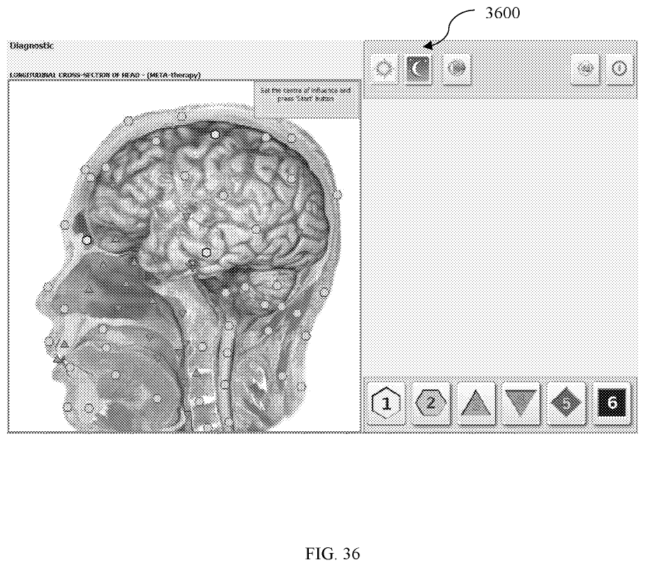

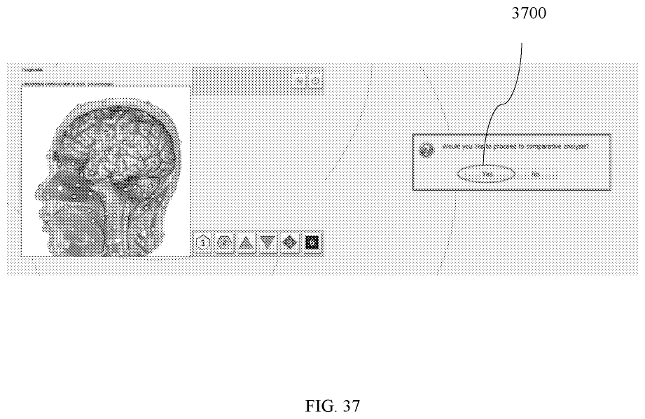

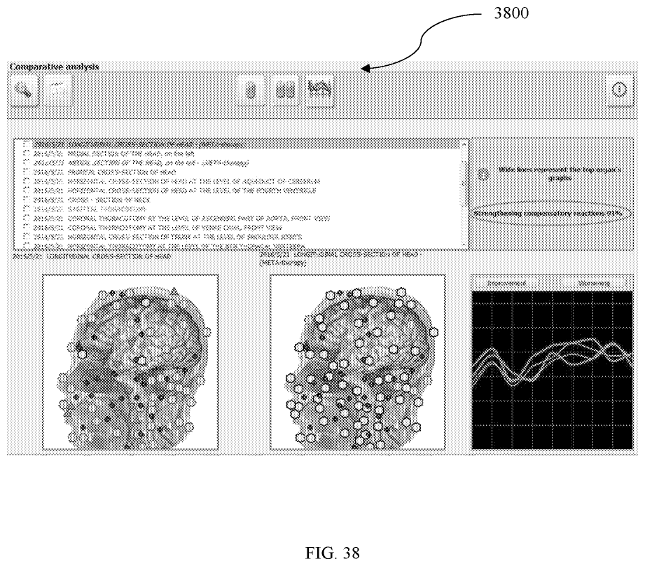

Further, FIG. 36 shows “Meta-Therapy” diagnostics 3600. It may be noted that, stress within a person causes a block in the flow of energy, which in turn causes disease and illness. META-Therapy helps to remove these blocks, allowing the body to heal itself. META-Therapy is non-invasive, painless and strengthens the immune system. It's even safe for pregnant women and children. Upon clicking a “Start” button, meta-therapy will be started as described above and shown in FIG. 37. Further, as shown in FIG. 37 shows upon clicking the “Yes” button 3700, the user interface 110 shows comparative analysis result in improvement or worsening as shown by 3800 in FIG. 38.

In one example, strengthening of compensatory reactions to 91%. It means the general improvement in the second case in comparison with the first. The user may observe the influence in details be looked by pressing Improvement and Worsening to the right of the pictures. When the user clicks the Improvement the points with parameters very close to the standard will start to blink on the pictures of an organ. After pressing, worsening the points which have significant deviation from the standard will blink. Thus, influence of this medicine has weakened compensatory reactions of the organ in these parts. It is necessary to carry out additional examination for selection of other standard which will compensate weakening. (We know that drastic antibacterial medicines weaken digestive and immune systems. Therefore, compensatory medicines, probiotics and prebiotics are prescribed together with them) . Also, it is possible to compare the results of influence with the help of curves of the possesses. TESTS shows green crosses on pictures of organs with the corresponding description of parts of organs. At the bottom part of the screen, the user may see the name of the chosen organ. EXIT To finish user's work with the comparative analysis menu.

The META-therapy helps the body to better deal with the discovered problems, toxins and allergens and to eliminate these. In addition, the higher causal factors of organic disturbance chronic stress and disturbed reactions of the central nervous system will be neutralized due to the therapy. Both are important factors for the activation of the immune system and self-healing, enabling the healing of even very old organ diseases. The course of treatment: 5~6 organs for 1 sessions every other days, totally 10 sessions in 25 days. For therapy session: Need about 15~20 minutes. The time need be repeated depends on the nature of the illness. Usually need 5~10 sessions. For preventive care: It is recommended 1 session every other days, totally 10 sessions. If want to make Meta-therapy again, suggest to relax for one month. When treat for diseases, 1 session every day, treat for 5-6 organs for 1 session, suggest to relax for 2~3 weeks, then the user may treat again.

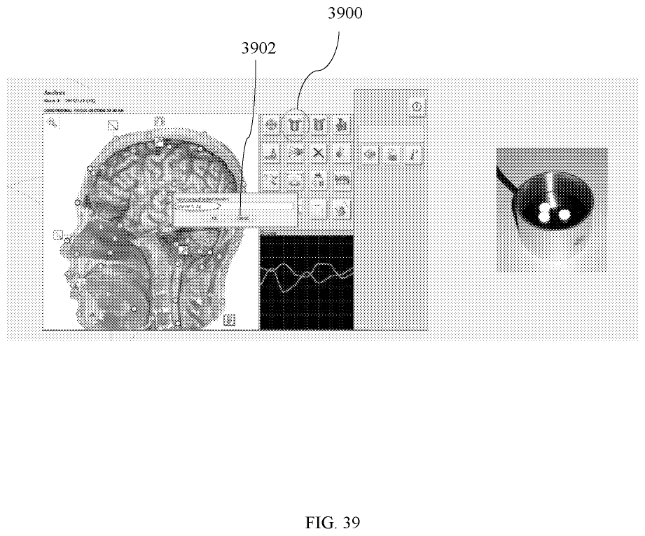

Further, FIG. 39 shows Vegeto Test (External) where upon clicking on “Vegeto Test” button 3900, the user is required to input name of tested standard for example “Vitamin C 3 g”. The given button allows to carry out external Vegeto test with the use of the resonant chamber to check the efficiency of standards which were not included into the matrix. The device database contains a considerable quantity of etalons and the device can simulate the results of their application and possible negative consequences. To check the efficiency of medicine which is not in the catalogue of the program put it into the resonant chamber and click the Vegeto test. Then, the user may be suggested to put down the name of the etalon into the entry line of the open window. Next, click OK, and testing will begin automatically. Further, the user may click “Cancel” button 3902 if user wish to leave the menu. Further the user may use the comparative analysis to evaluate efficiency of the given medicine for this organ. Compare the virtual model of the organ during research to the virtual model of the organ after the use of etalon, and evaluate strengthening/weakening compensatory reactions of the organism. If the user may wish to test the etalon from the catalogue of the program, then first enter the window Test Standard and then in this window choose Vegeto test.

One of the more popular uses of the Metatron is the “Vegeto test”. The control of the vegetative resonance brings understandable ideas and news of the manner in which various medicines can affect the human body. As known that the material world possesses frequencies just as the human body. These frequencies contain an information and this information can be read by the system. The frequencies of a medication can get into harmony with a person or not. This idea is transformed into the principle of resonance. The closer the frequency of the medication is to our frequency, the more efficient it is. That is why it is important to choose alimentary additives, homeopathic formula, allopathic remedies, vitamins and plants (phyto-aroma) according to the principle of resonance. The Vegeto control may be used to verify the adjustment of a medication to the problem organ of a person. There are two ways to proceed. Either by comparison of a pre-existing medicine found in the given database, or by verifying the relevance of a medication the patient already uses.

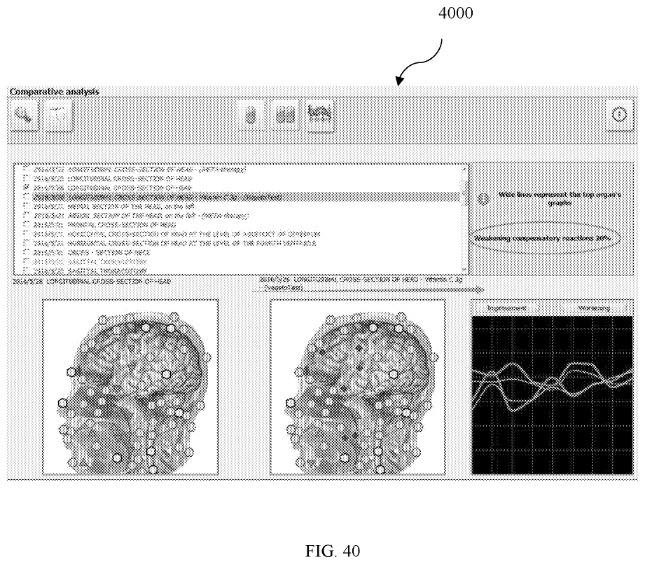

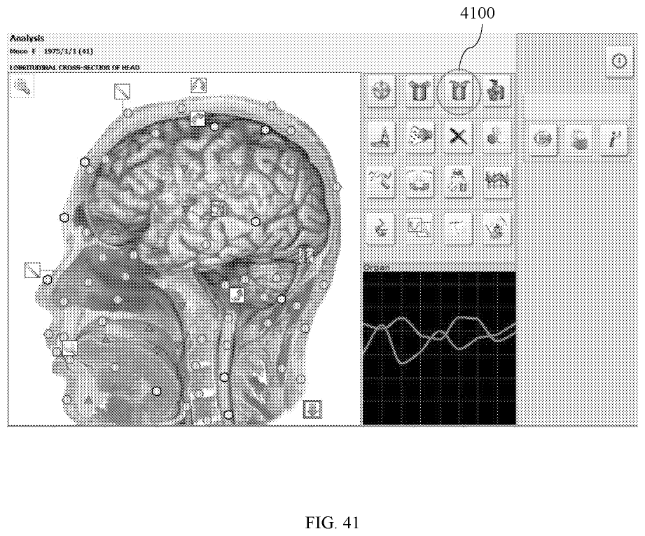

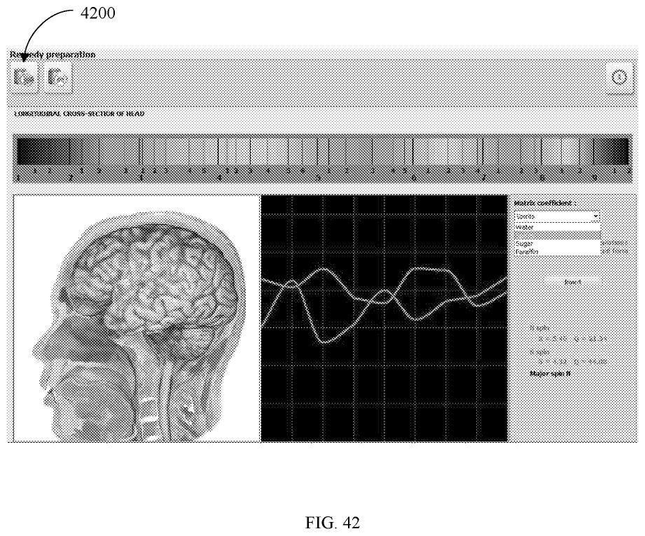

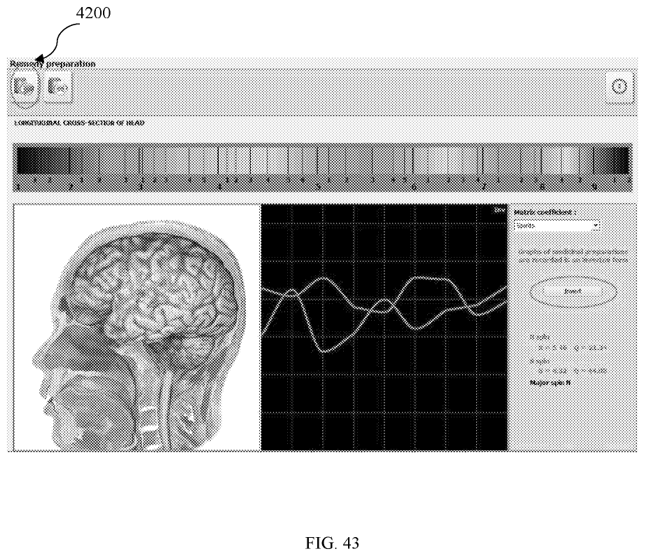

As shown in FIG. 40, 4000, shows the comparative analysis result in improvement or worsening by using the vitamin C 3 g. Further, as shown in FIG. 41, a “Reprinter” button 4100 may be clicked by the user that allows the user to put down the information about etalons on various carriers as shown in FIG. 42. An other variation of the function ‘Reprinter’, is the emission of various medication frequencies into the liquid. The given database contains frequencies of various alimentary additives, homeopathic formula, phyto and allopathic remedies, all placed in inverted form. It is possible to send (Reprinter) these into a liquid in a manner identical with the organs. It is thus possible to arrive at an individual ‘liquid solution’ exclusive to this person. It is possible to charge up to five different frequencies into the same bottle. The medication will find the organ appropriate to its resonance, which can be disturbed if there are more than five medications in use. The ideal would be to use a container for each organ, charged with only one frequency. Thus, if user want to treat the heart, the liver and the kidneys, the patient can have three different containers to be used individually at the time when the organ is most active according to the Chinese energetic hour clock. However, such a way to take the medicines is not always practical at the right moment, and it is then simpler and more polyvalent to charge all the frequencies into one bottle.

Further, the user may choose a necessary medicine effective for the given patient (index D<0.425=a medicine in the red zone) and click the Reprinter button 4200. It is important to remember that all medicines are written down in a database in the inverted way, therefore before the record begins it is necessary to click the button invert. Then click Start preparation. For the urgent interruption of the process of manufacturing click the button Stop preparation. When the data recording on the carrier is finished, the program will stop automatically. To finish the work with Reprinter click the button Exit.

In some embodiments, efficiency of the given medicines differs depending on a matrix water. For this purpose, it is advised to be used to cure acute processes. Preparation on the water is effective 2-3 weeks ALCOHOL Medicine made on the basis of it is effective up to 2-3 months. SUGAR Medicine on the basis of dairy is effective for 6-9 months and is used for treatment of chronic diseases. One dose of a medicine for acute processes usually makes 4-8 drops or grains 3 times a day. For children under the age of 14 use 2-3 drops. PARAFFIN Medicine made on the basis of it is used for treatment of skin and neurologic diseases. (eczema, radiculitis, neuralgia, etc) For treatment of chronic processes simultaneous record of several medicines (2-4) on one carrier is possible.

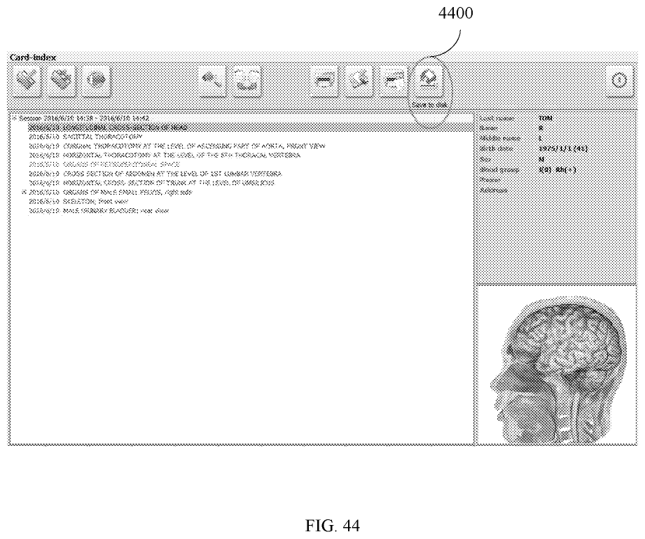

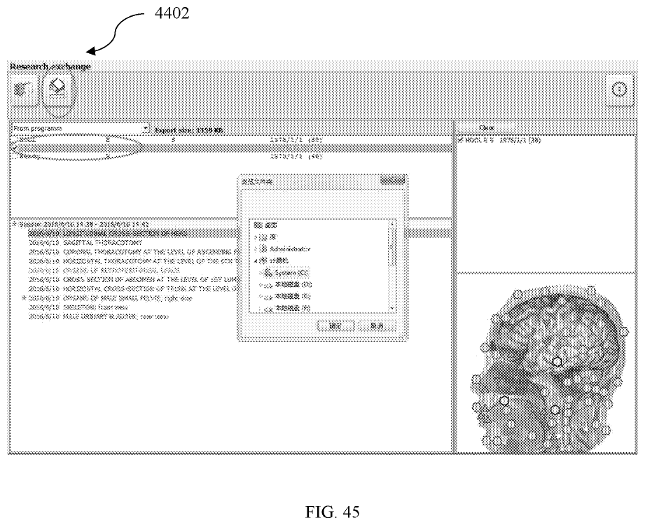

For treatment of chronic processes, the quantity of medicines can be increased to 6-8. All materials (sugar, medicines, paraffin, etc) should be put into Reprinter papered or poured in a glass flask. Otherwise, molecules of these substances may deposit in reprinter which can influence on the future results and worsen the effectiveness of medicine. Please wipe the Reprinter with a dry rag only and avoid water in it. For treatment of chronic processes simultaneous record of several medicines (2-4) on one carrier is possible. For treatment of chronic processes, the quantity of medicines can be increased to 6-8. All materials (sugar, medicines, paraffin, etc) should be put into Reprinter papered or poured in a glass flask. Otherwise, molecules of these substances may deposit in reprinter which can influence on the future results and worsen the effectiveness of medicine. Please wipe the Reprinter with a dry rag only and avoid water in it. Further, as shown in FIG. 44, the user after finishing all the task may click on a “Save to Disk” button 4400. Further, the user may click on the client required and click “Copy to disk” button 4402 to save the client as shown in FIG. 45.

In one example embodiment, a personalized health optimization plan is developed for Ms. Angel Acosta utilizing the diagnostic system's advanced bioelectrical analysis capabilities. The system's entropy-based two-factor analysis identified spectral similarities between Ms. Acosta's current physiological state and predefined pathological models, revealing a moderate to high entropy dispersion across various organ systems. Notably, the nervous system exhibited balanced vegetative tone (eutonia) and normergic response, while the musculoskeletal system displayed moderate degeneration, including second-to third-degree disc dehydration and thoracic disk herniation. The digestive system showed moderate dysfunction across multiple organs, and the urinary system presented with moderate dysfunction involving the bladder, kidneys, and prostate. Additionally, the analysis indicated mild hyperfunction in the immune and lymphatic systems, with elevated activity in the tonsils, cervical/mediastinal lymph nodes, and thymus.

The pathogen and toxin load assessment revealed a high probability of presence of various microorganisms, including Peptococcus variabilis, Chlamydia psittaci, Acinetobacter lwoffii, human alphaherpesvirus 1, Thottapalayam orthohantavirus, Geotrichium candidum, Dientamoeba fragilis, Eimeria, and Opisthorchis viverrini. Vitamin deficiency analysis indicated poor absorption of B-complex vitamins, Vitamin D, F, H (Biotin), and P. Furthermore, the system's analysis suggested a biological age approximately one year older than chronological age, attributed to lifestyle and environmental exposures. In the realm of mental clarity and emotional wellness, the system's biodecodification scan identified specific somatic memory loads affecting various organs, including the kidneys, stomach, pancreas, intestines, and female reproductive organs. These findings provided a basis for targeted emotional therapies aimed at addressing and clearing emotional blocks associated with these organs.

Based on these comprehensive analyses, a personalized action plan was formulated, encompassing three phases:

### Phase 1 (Weeks 1-4): Drainage & Detox Support

Objective: Open drainage pathways, initiate detoxification, and support gut health. Recommendations included the use of BPC-157 (7 troches), CellCore Detox & Drainage Support (CT-Minerals, KL Support, BC-ATP, BioToxin Binder), with a total phase cost of $540.

### Phase 3 (Weeks 9-12): Nervous System, Mitochondrial & Metabolic Recovery

Objective: Repair neuroendocrine system, restore mitochondrial energy, and support spine and stress recovery. Recommendations included continued use of BPC-157, addition of GHK-Cu & Epitalon (5 ml vial), and ongoing CellCore Detox & Drainage Support, with a total phase cost of $799.

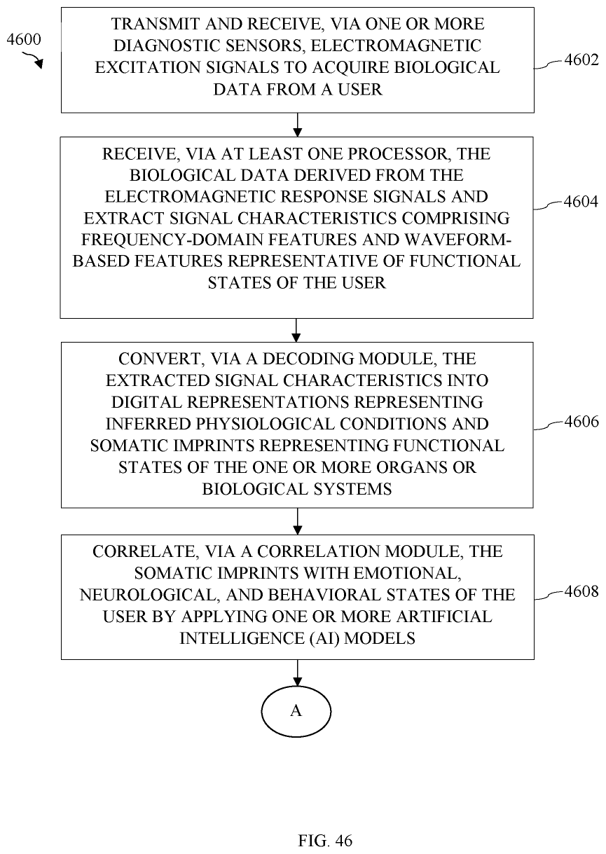

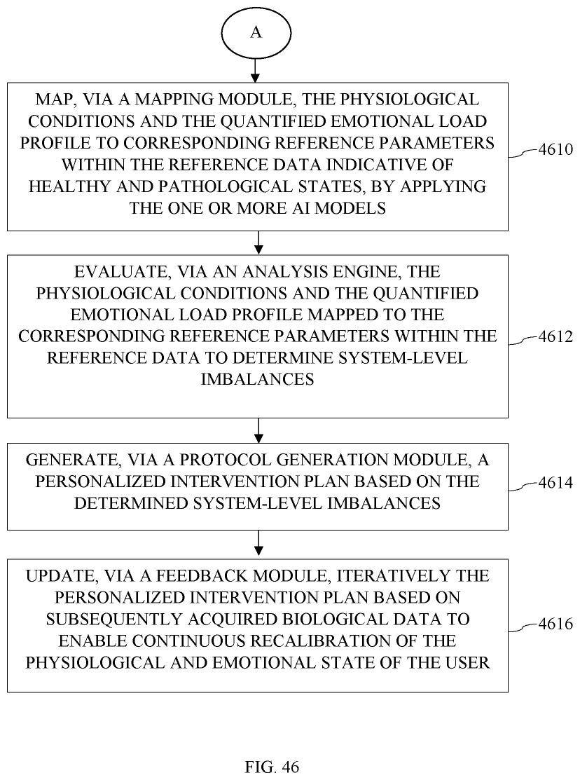

FIG. 46 illustrates a flowchart showing a computer-implemented method 4600 for biometric analysis, according to an embodiment of the present disclosure.

At step 4602, the one or more diagnostic sensors 108 is configured transmitting and receiving electromagnetic excitation signals to acquire biological data from the user, the biological data comprising at least bioelectrical activity, electromagnetic wave characteristics, biofrequency patterns, and physiological parameters associated with physiological activity of one or more tissues, one or more organs, and cellular activity. In some embodiments, the acquired biological data comprises bioelectrical activity indicative of cellular membrane potentials and electrochemical signaling, electromagnetic wave characteristics corresponding to tissue-specific resonance and propagation properties, biofrequency patterns representing oscillatory behavior across one or more biological systems, and physiological parameters associated with physiological activity of one or more tissues, one or more organs, and cellular activity.

In some embodiments, the acquisition of biological data is performed in a non-contact or near-field manner, wherein the one or more diagnostic sensors 108 detect electromagnetic emissions or responses without requiring direct physical contact with the user. The biological data may be acquired continuously, periodically, or in response to a user-initiated measurement event. In some embodiments, the acquired biological data includes multi-dimensional signal information across a range of frequencies and amplitudes, enabling capture of both steady-state and dynamic variations in physiological activity. The biological data may further reflect spatial variations across different anatomical regions, including localized signal deviations associated with specific tissues, organs, or fascial structures.

In an example embodiment, a user may wear a wearable biometric analysis device configured as a smartwatch or a head-mounted device positioned in proximity to the user's body. Upon initiation of a measurement session via a user interface, the one or more diagnostic sensors 108 transmit low-energy electromagnetic excitation signals toward the user and receive corresponding electromagnetic responses influenced by biological tissues. In this embodiment, the diagnostic sensors 108 acquire biological data comprising bioelectrical activity, biofrequency patterns, and electromagnetic wave characteristics associated with physiological activity of one or more organs and biological systems of the user. The acquired biological data may reflect physiological conditions including variations in autonomic nervous system activity, metabolic function, and tissue-level electrical behavior. The biological data is then communicated to the at least one processor 200 for subsequent processing, including extraction of signal characteristics and determination of functional states of the user in accordance with the subsequent steps of the method.

At step 4604, the at least one processor 200 is configured to receive the biological data derived from the electromagnetic response signals and extract signal characteristics comprising frequency-domain features and waveform-based features representative of functional states of the user using one or more signal transformation techniques. In some embodiments, the at least one processor 200 is configured to perform preprocessing operations on the biological data, including filtering, denoising, normalization, and segmentation, to isolate relevant signal components and reduce background interference. The preprocessing may include removal of statistical fluctuations, compensation for signal distortions, and enhancement of signal-to-noise ratio to enable accurate extraction of signal characteristics.

In some embodiments, the extraction of frequency-domain features comprises transforming the biological data into a frequency representation using one or more signal transformation techniques, including Fourier transform, wavelet transform, or spectral decomposition methods. The frequency-domain features may include spectral components, resonance peaks, harmonic structures, and frequency deviations indicative of physiological conditions associated with physiological activity of one or more tissues or organs. In some embodiments, the extraction of waveform-based features comprises analyzing time-domain characteristics of the biological data, including amplitude variations, phase relationships, temporal patterns, signal morphology, and transient responses. The waveform-based features may capture dynamic physiological behavior, including variations associated with autonomic nervous system activity, metabolic fluctuations, and localized tissue responses.

In some embodiments, the extracted signal characteristics collectively represent functional states of the user by encoding information related to physiological regulation, including autonomic balance, inflammatory activity, metabolic efficiency, and recovery capacity. The extracted signal characteristics may be further structured for subsequent processing in downstream steps of the method. In the example embodiment described in step 4602, the biological data acquired from the user is received by the at least one processor 200 for further processing. The at least one processor 200 performs preprocessing operations on the received biological data to remove noise and isolate relevant signal components.

The at least one processor 200 then extracts frequency-domain features from the biological data, including dominant frequency components and resonance characteristics associated with physiological activity of one or more organs of the user. In addition, the at least one processor 200 extracts waveform-based features including amplitude variations and temporal patterns indicative of dynamic physiological activity. In this embodiment, the extracted signal characteristics represent functional states of the user, including indicators associated with autonomic nervous system activity, metabolic function, and recovery capacity, and are subsequently used for generating physiological conditions and somatic imprints in the following steps of the method.

At step 4606, the at least one processor 200 is configured to convert, via the decoding module 206 communicatively coupled to the at least one processor 200, the extracted signal characteristics into digital representations representing inferred physiological conditions and somatic imprints representing inferred functional states of the one or more tissues, the one or more organs or biological systems based on the extracted signal characteristics. In some embodiments, the at least one processor 200, via the decoding module 206, is configured to interpret the extracted signal characteristics to identify patterns, deviations, and resonance behaviors associated with physiological activity of one or more tissues, one or more organs, or biological systems. The conversion may include mapping spectral components, frequency deviations, and waveform characteristics to corresponding physiological conditions, including indicators of inflammation, metabolic imbalance, autonomic nervous system activity, and recovery capacity.

In some embodiments, the at least one processor 200 is configured to apply one or more artificial intelligence models 204 to infer relationships between the extracted signal characteristics and the physiological conditions. The one or more artificial intelligence models 204 may be trained using reference datasets comprising associations between biofrequency patterns and known biological states, thereby enabling inference of physiological conditions based on the extracted signal characteristics. In some embodiments, the digital representations comprise structured data formats including feature vectors, parameter sets, or encoded state variables representing functional conditions of the one or more organs or biological systems. The digital representations may further include spatial or regional information corresponding to anatomical locations.

In some embodiments, the somatic imprints comprise data representations derived from the digital representations that correspond to localized or system-level patterns of physiological deviation. The somatic imprints may represent functional states associated with specific tissues or organs, including patterns influenced by autonomic dysregulation, metabolic conditions, or persistent physiological or emotional influences. In some embodiments, the digital representations and the somatic imprints form an intermediate representation layer for subsequent correlation with emotional, neurological, and behavioral states of the user. In the example embodiment described in step 4604, the extracted signal characteristics are provided to the decoding module 206 for further processing. The at least one processor 200, via the decoding module 206, converts the extracted signal characteristics into digital representations corresponding to physiological conditions of the user.

In this embodiment, the conversion includes identifying resonance patterns and frequency deviations associated with physiological activity of one or more organs, and mapping such patterns to physiological conditions including indicators of stress response, metabolic activity, and inflammatory processes. The at least one processor 200 further applies one or more artificial intelligence models 204 to infer relationships between the extracted signal characteristics and the physiological conditions. The decoding module 206 generates somatic imprints representing functional states of the one or more organs or biological systems, including localized representations of physiological deviation associated with specific anatomical regions. The generated digital representations and somatic imprints are subsequently used for correlation with emotional and neurological states in the following step.

At step 4608, the at least one processor 200 is configured to correlating, via a correlation module communicatively coupled to the at least one processor, the somatic imprints with emotional, neurological, and behavioral states of the user by applying one or more artificial intelligence (AI) models trained on reference data comprising physiological signal patterns and corresponding functional states, and generate a quantified emotional load profile computed from physiological signal variations corresponding to autonomic nervous system activity. In some embodiments, the at least one processor 200 is configured to apply the one or more artificial intelligence models to identify relationships between physiological conditions of the one or more organs or biological systems and emotional, neurological, and behavioral states. The one or more artificial intelligence models may be trained using datasets comprising associations between biofrequency patterns, autonomic nervous system responses, and emotional conditions, thereby enabling inference of emotional states from the somatic imprints.

In some embodiments, the correlating comprises identifying patterns associated with emotional states including stress, anxiety, depressive states, heightened arousal, reduced regulatory capacity, or other emotional or neurological conditions, wherein such states are inferred based on physiological representations encoded in the somatic imprints. The correlating may further account for interactions between physiological conditions and autonomic nervous system activity, thereby linking physiological dysregulation with emotional and behavioral manifestations. In some embodiments, the quantified emotional load profile comprises a structured representation of emotional and neurological burden associated with the user, including numerical values, weighted scores, multi-dimensional vectors, or probabilistic measures representing intensity, distribution, and relative contribution of different emotional states across the one or more organs or biological systems. The quantified emotional load profile may further include spatial and temporal components corresponding to localized or time-varying emotional load.

In some embodiments, the quantified emotional load profile provides an integrated representation of interactions between physiological conditions and emotional states, thereby enabling identification of systemic patterns of dysregulation arising from sustained emotional or neurological influences. In the example embodiment described in step 4606, the somatic imprints generated by the decoding module 206 are provided to the correlation module for further processing. The at least one processor 200 applies one or more artificial intelligence models to correlate the somatic imprints with emotional, neurological, and behavioral states of the user. In some embodiments, the system is configured to generate behavioral or neuro-regulatory intervention recommendations based on the quantified emotional load profile and inferred stress-response patterns.

In this embodiment, the at least one processor 200 identifies relationships between physiological conditions associated with physiological activity of one or more organs and corresponding emotional states, including indicators of stress response and reduced regulatory capacity. The artificial intelligence models infer emotional states based on patterns present in the somatic imprints, including patterns associated with autonomic nervous system activity. The at least one processor 200 generates a quantified emotional load profile comprising weighted scores representing intensity and distribution of emotional states across the one or more organs or biological systems. The quantified emotional load profile is subsequently used in combination with the physiological conditions for further processing in the following steps of the method.

At step 4610, the at least one processor 200 is configured to map, via the mapping module 210 communicatively coupled to the at least one processor 200, the physiological conditions and the quantified emotional load profile to corresponding reference parameters within the reference data indicative of healthy and pathological states, by applying the one or more AI models configured to learn relationships between the physiological conditions, emotional load factors, and the reference parameters. In some embodiments, the biometric analysis system 100 is configured to generate a multi-organ representation comprising a plurality of organ-level or system-level indicators, wherein each indicator corresponds to a relative functional condition inferred from the biological data.

In some embodiments, the at least one processor 200 applies the one or more AI models to determine relationships between multi-dimensional inputs comprising physiological conditions and quantified emotional load factors, and corresponding parameters within the reference data. The one or more AI models may be trained to learn associations between combinations of physiological signals, emotional states, and known healthy or pathological conditions, thereby enabling identification of relevant subsets of reference parameters for a given user state. In some embodiments, the mapping comprises performing feature relevance determination, parameter selection, and weighting of reference parameters based on combined influence of physiological conditions and emotional load factors. The mapping may therefore include assigning weights or confidence scores to different reference parameters, prioritizing certain parameters over others, and dynamically selecting those reference parameters that are most indicative of the current system state of the user.

In some embodiments, the mapping further comprises identifying non-linear relationships between physiological conditions, emotional load factors, and reference parameters, including interactions across multiple organ systems and regulatory pathways. The mapping may therefore account for interdependencies between physiological and emotional domains to enable context-aware alignment of input data with the reference data. In some embodiments, the mapping results in generation of mapped representations, wherein the physiological conditions and the quantified emotional load profile are transformed into a reference-aligned format corresponding to selected reference parameters, thereby enabling targeted and efficient evaluation in subsequent steps.

In the example embodiment described in step 4608, the physiological conditions and the quantified emotional load profile are provided to the mapping module 210 for further processing. The at least one processor 200 applies one or more artificial intelligence models to map the physiological conditions and the quantified emotional load profile to corresponding reference parameters within the reference data indicative of healthy and pathological states. In this embodiment, the one or more artificial intelligence models determine relationships between the physiological conditions and emotional load factors and select relevant reference parameters corresponding to specific organ systems and regulatory functions. The mapping includes assigning weights to the selected reference parameters based on the combined influence of physiological and emotional factors, thereby prioritizing the most relevant reference criteria for subsequent evaluation. The at least one processor 200 generates mapped representations corresponding to the selected reference parameters, which are subsequently used for determining system-level imbalances in the following step. In some embodiments, the at least one processor 200 is configured to determine probabilistic indicators of physiological conditions based on statistical relationships between signal characteristics and reference data.

At step 4612, the at least one processor 200 is configured to evaluate, via the analysis engine 212 communicatively coupled to the at least one processor 200, the physiological conditions and the quantified emotional load profile mapped to the corresponding reference parameters within the reference data to determine system-level imbalances. In some embodiments, the analysis engine 212 is configured to generate system-level scores corresponding to one or more physiological systems of the user, wherein each score represents a relative functional state derived from the processed biological data. In some embodiments, the at least one processor 200 is configured to classify physiological conditions into relative functional states comprising at least one of elevated activity, reduced activity, or nominal condition, based on deviations of signal characteristics from reference parameters. In some embodiments, the analysis engine 212 is configured to rank the plurality of physiological conditions or system-level imbalances based on severity or deviation from reference parameters, thereby prioritizing intervention targets. In some embodiments, the biometric analysis system 100 is configured to derive composite health metrics comprising at least one of biological age indicators, system resilience indicators, or recovery capacity metrics based on the evaluated physiological conditions.

In some embodiments, the at least one processor 200 is configured to infer potential physiological load conditions associated with environmental, metabolic, or biochemical stressors based on patterns in the biological data. In some embodiments, the at least one processor 200 determines system-level imbalances by identifying variations in frequency characteristics, spectral distributions, and physiological indicators relative to the reference data. Such variations may correspond to dysregulation in biological systems, including elevated inflammatory activity, altered autonomic nervous system balance, impaired metabolic function, or reduced physiological recovery capacity. In some embodiments, the determination of inflammation comprises identifying signal patterns indicative of tissue-level stress responses, immune activation, or deviations in bioelectrical characteristics associated with inflammatory processes. The determination of stress response comprises analyzing signal characteristics associated with autonomic nervous system activity, including sympathetic activation or parasympathetic suppression.

In some embodiments, the determination of metabolic efficiency comprises evaluating signal characteristics associated with cellular energy utilization, electrochemical activity, and tissue-level metabolic function inferred from the physiological conditions. The determination of recovery capacity comprises assessing the ability of the physiological systems of the user to return to baseline states following perturbations, as indicated by temporal variations and regulatory patterns in the biological data. In some embodiments, the evaluating integrates both the physiological conditions and the quantified emotional load profile, thereby enabling identification of system-level imbalances arising from interactions between physiological and emotional factors.

In the example embodiment described in step 4610, the mapped representations of the physiological conditions and the quantified emotional load profile are provided to the analysis engine 212 for evaluation. The at least one processor 200 compares the mapped representations with corresponding reference parameters indicative of healthy and pathological states. In this embodiment, the at least one processor 200 identifies deviations from reference conditions associated with physiological activity of one or more organs and biological systems, including indicators of elevated inflammatory activity, increased stress response, reduced metabolic efficiency, and impaired recovery capacity. The evaluation further incorporates the quantified emotional load profile to account for interactions between physiological and emotional factors. The at least one processor 200 determines system-level imbalances based on the identified deviations, and generates evaluation outputs representing the functional state of the user, which are subsequently used for generating a personalized intervention plan in the following step.

At step 4614, the at least one processor 200 is configured to generate, via the protocol generation module 214 communicatively coupled to the at least one processor 200, the personalized intervention plan based on the determined system-level imbalances. In some embodiments, the personalized intervention plan comprises one or more intervention modalities selected based on the type, severity, and distribution of the system-level imbalances. The intervention modalities may include, without limitation, biochemical interventions, nutritional supplementation, compounded formulations, peptide-based therapies, neuromodulation techniques, behavioral modifications, activity and lifestyle adjustments, and mind-body or somatic therapies. In some embodiments, the personalized intervention plan comprises recommendations associated with nutritional or biochemical support based on inferred physiological requirements derived from the biological data.

In some embodiments, the protocol generation module 214 is configured to select intervention strategies from a predefined library of protocols, wherein the selection is based on the evaluated physiological conditions and system-level imbalances. In some embodiments, the personalized intervention plan is configured to address both physiological and emotional components of the system-level imbalances by integrating multi-domain interventions targeting biological regulation, neurological function, and emotional patterning. In some embodiments, the personalized intervention plan comprises a multi-stage framework including a reset phase configured to reduce inflammatory load and physiological interference signals, a replenish phase configured to provide personalized biochemical and nutritional support, a regulate phase configured to modify neural, emotional, and behavioral response patterns, and a performance phase configured to enhance physiological and cognitive function.

In some embodiments, the generating further comprises determining sequencing, prioritization, and progression of the intervention protocols based on the severity and interdependency of the system-level imbalances, such that the personalized intervention plan is applied in a structured and adaptive manner. In some embodiments, the personalized intervention plan further comprises actionable recommendations including schedules, dosages, durations, and implementation guidance for execution by the user. In the example embodiment described in step 4612, the determined system-level imbalances are provided to the protocol generation module 214 for generation of the personalized intervention plan. The at least one processor 200 selects one or more intervention modalities based on the identified imbalances, including interventions targeting inflammation, stress response, metabolic function, and recovery capacity.

In this embodiment, the at least one processor 200 generates a personalized intervention plan comprising a multi-stage framework including a reset phase to reduce inflammatory load, a replenish phase to provide biochemical and nutritional support, a regulate phase to modify neural and emotional response patterns, and a performance phase to enhance physiological and cognitive function. The personalized intervention plan further includes structured recommendations including specific intervention protocols, sequencing of phases, and implementation parameters tailored to the physiological conditions and quantified emotional load profile of the user. The generated personalized intervention plan is subsequently provided for iterative updating in the following step.

At step 4616, the at least one processor 200 is configured to update, via the feedback module 216 communicatively coupled to the at least one processor 200, iteratively the personalized intervention plan based on subsequently acquired biological data to enable continuous recalibration of the physiological and emotional state of the user. In some embodiments, the iterative updating comprises modifying one or more aspects of the personalized intervention plan, including adjustment of intervention modalities, modification of dosages or intensity, reordering of intervention phases, or introduction of additional intervention protocols based on updated physiological conditions and quantified emotional load profile.

In some embodiments, the at least one processor 200 implements a closed-loop feedback mechanism, wherein execution of the personalized intervention plan influences subsequent biological data, and the subsequently acquired biological data is used to further refine the personalized intervention plan. The closed-loop system enables adaptive optimization of the intervention plan based on real-time or near real-time feedback from the user's biological system. In some embodiments, the updating further comprises tracking longitudinal changes in the physiological conditions and the quantified emotional load profile over time, thereby enabling monitoring of trends associated with recovery, regulation, or progression of system-level imbalances. The method described herein represents a signal processing and artificial intelligence-driven pipeline implementing a closed-loop feedback mechanism for continuous monitoring and optimization of physiological and emotional states.

In some embodiments, the continuous recalibration enables the personalized intervention plan to remain dynamically aligned with the evolving physiological and emotional state of the user, thereby supporting sustained improvement in system performance, including reduction in inflammation, stabilization of emotional states, improvement in metabolic efficiency, and enhancement of recovery capacity. In the example embodiment described in step 4614, the personalized intervention plan is implemented by the user, and subsequently acquired biological data is obtained via the one or more diagnostic sensors 108. The at least one processor 200 processes the subsequently acquired biological data to generate updated physiological conditions and quantified emotional load profile.

In this embodiment, the at least one processor 200 updates the personalized intervention plan by adjusting intervention modalities, modifying sequencing of phases, and refining implementation parameters based on the updated system-level imbalances. The feedback module 216 enables continuous recalibration of the personalized intervention plan in response to changes in the physiological and emotional state of the user. The iterative updating process is repeated over time, thereby forming a closed-loop system that continuously adapts the personalized intervention plan to improve physiological regulation and emotional balance of the user.

In some embodiments, the personalized intervention plan generated by the at least one processor 200 is structured according to a multi-stage homeostasis protocol comprising a plurality of sequential stages including a reset stage, a replenish stage, a regulate stage, and a performance stage, wherein the stages are determined based on the physiological conditions, the somatic imprints, and the quantified emotional load profile. In some embodiments, the multi-stage homeostasis protocol is dynamically generated and adapted by the at least one processor 200 based on system-level imbalances and recovery metrics, such that progression between stages and composition of each stage is personalized for the user.

In some embodiments, the biometric analysis system 100 is configured as an integrated system for simultaneous evaluation and correction of physiological, emotional, neurological, and behavioral domains of the user, wherein the at least one processor 200 is configured to model interactions between the domains and generate coordinated intervention strategies addressing the domains in combination rather than independently. In some embodiments, the reset stage is configured based on the determined system-level imbalances to reduce physiological interference signals, inflammatory load, and biological congestion identified from the biological data, wherein the reset stage comprises targeted detoxification protocols, microbiome balancing, and reduction of pathogenic or toxic influences corresponding to the physiological conditions.

In some embodiments, the replenish stage comprises generation of a personalized formulation comprising a plurality of compounds selected based on the physiological conditions and the quantified emotional load profile, wherein the at least one processor 200 determines composition, dosage, and combination of the plurality of compounds based on identified deficiencies, metabolic conditions, and system-level imbalances. In some embodiments, the regulate stage comprises modification of neural, emotional, and behavioral response patterns based on the quantified emotional load profile, wherein the correlation module 208 is configured to identify subconscious patterns, emotional imprints, and nervous-system dysregulation associated with the user, and the personalized intervention plan includes structured neural retraining protocols configured to alter such patterns.

In some embodiments, the personalized intervention plan comprises a plurality of structured neural retraining sessions configured to modify neural circuitry associated with emotional and behavioral responses, wherein the neural retraining sessions are configured to reduce maladaptive stress responses, eliminate persistent emotional patterns, and improve regulatory capacity of the autonomic nervous system. In some embodiments, the performance stage is configured to enhance physiological and cognitive function following regulation of system-level imbalances, wherein the performance stage comprises advanced optimization protocols including peptide-based therapies, regenerative interventions, metabolic enhancement, and performance optimization strategies selected based on system performance metrics.

In some embodiments, the at least one processor 200 is configured to generate the personalized intervention plan based on system-level imbalances rather than isolated symptoms or individual biomarkers, wherein the system-level imbalances represent integrated deviations across physiological, emotional, and neurological domains. In some embodiments, execution of the multi-stage homeostasis protocol is continuously adapted through the feedback module 216 based on subsequently acquired biological data, such that the reset stage, replenish stage, regulate stage, and performance stage are iteratively refined to improve system performance and recovery metrics of the user.

In some embodiments, the at least one processor 200 is configured to generate a structured representation of the user's physiological conditions, somatic imprints, and quantified emotional load profile, wherein the structured representation comprises a precision biological blueprint of the user. The precision biological blueprint may represent a system-level mapping of functional states, imbalances, and interdependencies across one or more biological systems, and may be used to guide generation and adaptation of the personalized intervention plan.

In some embodiments, the at least one processor 200 is configured to determine that somatic imprints corresponding to emotional states influence one or more physiological conditions including sleep regulation, metabolic function, hormonal balance, digestive activity, and immune response. The at least one processor 200 may be configured to model causal relationships between the somatic imprints and the physiological conditions, such that the personalized intervention plan targets underlying emotional drivers of physiological dysregulation.

In some embodiments, the personalized intervention plan is updated at predefined intervals corresponding to periodic cycles, wherein each cycle comprises reassessment of the physiological conditions and the quantified emotional load profile and generation of an updated intervention plan. The predefined intervals may correspond to quarterly updates, and each update may include modification of formulations, intervention protocols, and sequencing of stages.

In some embodiments, the at least one processor 200 using the user interface 110 is configured to present categorized system-level findings corresponding to distinct physiological and functional domains, wherein the domains comprise at least one of general physiological condition, psychosomatic state, gastrointestinal function and microbiome-associated physiological state, micronutrient status, autonomic nervous system balance, and energy levels. The categorized findings may be derived from the physiological conditions and the quantified emotional load profile. In some embodiments, the at least one processor 200 is configured to sequence intervention stages such that physiological conditions associated with impaired absorption, gut-related physiological imbalance, or inflammatory load are addressed prior to implementation of replenishment protocols, thereby improving bioavailability and effectiveness of subsequent interventions.

In some embodiments, the personalized intervention plan comprises a personalized supplement formulation comprising ingredient-specific and dose-specific components selected based on the physiological conditions and the quantified emotional load profile, wherein the formulation is configured to exclude non-essential components and is periodically updated based on changes in the physiological conditions. In some embodiments, the neural retraining sessions comprise a plurality of sequential phases including an initial phase configured to reduce stress reactivity, an intermediate phase configured to establish cognitive and emotional stability, a consolidation phase configured to reinforce adaptive behavioral patterns, and an integration phase configured to sustain long-term regulation across real-world conditions.

In some embodiments, the at least one processor 200 is configured to determine that dysregulation of the autonomic nervous system contributes to one or more system-level imbalances including sleep disturbances, inflammatory activity, metabolic dysregulation, and emotional instability, and to prioritize interventions targeting nervous system regulation within the personalized intervention plan. In some embodiments, the personalized intervention plan comprises administration of peptide-based therapies via one or more delivery modalities including subcutaneous injection, transmucosal delivery, or intranasal delivery, wherein the delivery modality is selected based on target physiological systems, absorption characteristics, and intended functional outcomes.

In some embodiments, the personalized intervention plan further comprises one or more bio-optimization therapies including light-based therapy, electromagnetic field therapy, oxygen-based therapy, or lymphatic stimulation protocols, wherein such therapies are configured to enhance cellular energy production, circulation, and recovery processes. In some embodiments, the biometric analysis system 100 further comprises or is communicatively coupled to a wearable device configured to deliver electromagnetic or frequency-based signals to the user, wherein the wearable device is configured to modulate cellular activity, support physiological regulation, and complement the personalized intervention plan. In some embodiments, the personalized intervention plan further comprises external or topical interventions configured to support physiological regulation of the skin as a biological organ, wherein such interventions are selected based on the physiological conditions and system-level imbalances associated with detoxification and barrier function.

In accordance with the present disclosure, a biometric analysis system provides a unique, non-invasive, and analogue-free solution for real-time diagnostics that combines the power of electromagnetic wave detection, AI-based analysis, and quantum-level diagnostics. One of the primary advantages of the biometric analysis system is its ability to decode subtle biological signals from the body, providing comprehensive insights into both the physiological and emotional states of the patient. The integration of signal-based physiological analysis system with non-contact frequency analysis enables the detection of health imbalances that traditional diagnostic methods may overlook. By processing these signals through AI algorithms, the system can generate real-time 3D visualizations of the organs, providing doctors with a deeper understanding of a patient's health. The color-coded entropy visualization helps healthcare providers to easily identify areas of concern and offers a dynamic, personalized diagnostic approach that enhances patient care and health optimization strategies. The biometric analysis system 100 integrates a signal processing pipeline, artificial intelligence-based modeling, a personalization engine, and a closed-loop feedback mechanism to provide continuous, data-driven optimization of physiological and emotional states of the user.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims. For example, external plates, such as the pendant front and back plates may be connected to internal securing plates (e.g., back plate cap and back plate caps) in various ways not explicitly described. Additionally, elements as shown in the drawings, though not specifically mentioned in the present disclosure still comprise elements of the invention.

### LIST OF ELEMENTS

100 Biometric Analysis System
102 Network
104 Server
106 Device
108 One or more Diagnostic Sensors
110 User Interface
200 At least one processor
202 Memory
204 One or More Artificial Intelligence (AI) Models
206 Decoding Module
208 Correlation Module
210 Mapping Module
212 Analysis Engine
214 Protocol Generation Module
216 Feedback Module
300 Method
302 Step
304 Step
306 Step
308 Step
310 Step
312 Step
400 Launching program page
500 Select doctor
502 Color images
504 Card-index
600 Green color
602 Red line
604 Brown line
700 Control buttons
800 “Close card file” button
802 “Record Fees” button
804 “Change password” button
806 “Delete card file” button
808 “Delete research” button
900 “Delete by date” button
902 “Doctor Selection” button
904 “New” button
906 “Delete” button
1000 Statistic analysis
1100 Card index feature
1200 New card button
1202 Card button
1204 “Date filter” button
1206 Edit card
1300 research button
1302 Current analysis button
1304 “View the results” button
1306 Comparative analysis button
1308 Researches button
1310 Conclusion button
1312 “Print conclusion”
1314 save to disk
1400 New card button
1402 Research button
1500 “scan” function
1600 “scan” button
1700 “ectomies” button
1702 “switch to manula/auto choice” button
1704 “remedy preparation” button
1706 “Text mode” button
1708 “ACCUPUNCTURE” button
1710 “Select system” button
1712 “additional projection/ultra-structure switch” button
1714 Button
1900 “research” button
2002 “Next/Exit” button
2200 “META-THERAPY” button
2202 “VEGETO TEST” button
2204 “REPRINTER” button
2206 “MAKE METAZODEN” button
2208 “MORE PRECISE NIDU” button
2210 “CONTOUR ON NIDU” button
2212 “DELETE CONTOUR ON NIDU” button
2214 “IMAGES IN BW/COLOR” button
2216 “TEST STANDARD” button
2218 “RESULT OF INFLUENCE” button
2220 “SPECTRUM” button
2222 “ULTRASTRUCTURE” button
2224 “PICTOGRAMS” button
2226 “DESCRIPTION STRUCTURE” button
2228 “DESCRIPTION FUNCTION” button
2300 “ETALONS” button
2302 “SMART FILTER” button
2304 “VIRTUAL MODEL” button
2306 “ENTROPY ANALYSIS” button
2308 “NLS-ANALYSIS” button
2310 “ADJUSTMENT” button
2312 “ADJUST ALL” button
2314 “TO EPICRISIS” button
2316 “VIEW EPICRISIS” button
2318 “ETALON-OBJECT” button
2320 “BACTERIAL ANALYSIS” button
2322 “LITHOTHERAPY” button
2324 “PHYTOTHERAPY” button
2400 Black Box
2500 Group List
2600 Vegeto Test
2700 Entropy Analysis
2800 Systematic Entropy Changes
2900 All Button
3000 Dot
3100 Arrow
3200 NL Analysis Button
3300 Exit Button
3400 Result
3500 “VIEW EPICRISIS” button
3600 “Meta-Therapy” diagnostics
3700 “Yes” button
3800 Analysis Result in Improvement or Worsening
3900 “VEGET TEST” button
4000 Comparative Analysis Result
4100 “REPRINTER” button
4200 “REPRINTER” button
4400 “Save to disk” button
4402 “Copy to disk” button
4600 Method
4602 Step
4604 Step
4606 Step
4608 Step
4610 Step
4612 Step
4614 Step
4616 Step

What is claimed is:

1. A biometric analysis system comprising:
one or more diagnostic sensors integrated into a device configured to be positioned in proximity to a user, wherein the one or more diagnostic sensors are configured to transmit electromagnetic excitation signals within one or more predefined frequency ranges and receive corresponding electromagnetic response signals generated from interaction with the user to acquire biological data from the user, the biological data comprising at least bioelectrical activity, electromagnetic wave characteristics, biofrequency patterns, and physiological parameters associated with physiological activity of one or more tissues, one or more organs, and cellular activity; and
at least one processor communicatively coupled to the one or more diagnostic sensors and a memory storing instructions, wherein the at least one processor, upon executing the instructions, is configured to:
receive the biological data derived from the electromagnetic response signals and extract signal characteristics comprising frequency-domain features and waveform-based features representative of functional states of the user using one or more signal transformation techniques;
convert, via a decoding module communicatively coupled to the at least one processor, the extracted signal characteristics into digital representations representing inferred physiological conditions and somatic imprints representing inferred functional states of the one or more tissues, the one or more organs or biological systems based on the extracted signal characteristics;
correlate, via a correlation module communicatively coupled to the at least one processor, the somatic imprints with emotional, neurological, and behavioral states of the user by applying one or more artificial intelligence (AI) models trained on reference data comprising physiological signal patterns and corresponding functional states, and generate a quantified emotional load profile computed from physiological signal variations corresponding to autonomic nervous system activity;
map, via a mapping module communicatively coupled to the at least one processor, the physiological conditions and the quantified emotional load profile to corresponding reference parameters within the reference data indicative of healthy and pathological states, by applying the one or more AI models;
evaluate, via an analysis engine communicatively coupled to the at least one processor, the physiological conditions and the quantified emotional load profile mapped to the corresponding reference parameters within the reference data to determine system-level imbalances;
generate, via a protocol generation module communicatively coupled to the at least one processor, a personalized intervention plan based on the determined system-level imbalances; and
iteratively update, via a feedback module communicatively coupled to the at least one processor, the personalized intervention plan based on subsequently acquired biological data to enable continuous recalibration of the physiological and emotional state of the user.

2. The biometric analysis system of claim 1, wherein the one or more diagnostic sensors comprise electromagnetic sensors configured to transmit and receive the electromagnetic excitation signals and the electromagnetic response signals to acquire the biofrequency patterns corresponding to the physiological activity of the one or more tissues or the one or more organs of the user, wherein the one or more diagnostic sensors comprise at least one of an electromagnetic transceiver, a biofrequency sensor, a resonance-based sensor, or a bioimpedance sensor.

3. The biometric analysis system of claim 1, wherein the frequency-domain features comprise spectral components and resonance characteristics of the biological data derived from the electromagnetic response signals, and the waveform-based features comprise amplitude, phase, and temporal variations of the biological data, the frequency-domain features and the waveform-based features being indicative of functional states of the user, and the one or more signal transformation techniques comprises at least spectral decomposition, resonance characteristic identification, and detection of resonance peaks, impedance variations, and frequency response signatures corresponding to the physiological activity of the one or more tissues or the one or more organs.

4. The biometric analysis system of claim 1, wherein the somatic imprints comprise data representations derived from the extracted signal characteristics that correspond to functional conditions of the physiological activity of the one or more tissues, the one or more organs, or the biological systems of the user, and the system level imbalances comprises at least one of inflammation, stress response, metabolic efficiency, and recovery capacity based on deviations in the extracted signal characteristics.

5. The biometric analysis system of claim 1, wherein the device is configured as a wearable or user-adjacent device, wherein the device comprises a head-mounted device comprising at least one of headphones, earphones, earbuds, a headset, a wearable band, a smartwatch, a handheld device, or a computing device configured to be positioned in proximity to the user.

6. The biometric analysis system of claim 1, wherein the personalized intervention plan comprises a multi-stage framework for modifying the physiological and emotional state of the user.

7. The biometric analysis system of claim 6, wherein the multi-stage framework comprises:
a reset phase configured to reduce inflammatory load and physiological interference signals;
a replenish phase configured to provide personalized biochemical and nutritional support;
a regulate phase configured to modify neural and emotional response patterns; and
a performance phase configured to enhance physiological and cognitive function,
wherein the at least one processor enables the device to further generate a feedback signal comprising at least one electromagnetic or frequency-based output signal configured to be applied to the user, wherein the feedback signal is provided in accordance with the personalized intervention plan to modulate at least one physiological or neurological state of the user.

8. The biometric analysis system of claim 7, wherein the replenish phase comprises generating a personalized supplementation formulation including a combination of vitamins, minerals, botanicals, and nootropic compounds based on the system-level imbalances, wherein the personalized supplementation formulation comprises between 60 and 90 compounds.

9. The biometric analysis system of claim 7, wherein the regulate phase comprises one or more structured neural retraining sessions configured to modify subconscious emotional and behavioral patterns of the user, wherein the one or more structured neural retraining sessions comprise a predefined sequence of sessions conducted over a plurality of time intervals.

10. The biometric analysis system of claim 1, wherein the at least one processor, using a user interface operatively coupled to the at least one processor, is configured to present the physiological conditions, the quantified emotional load profile, and the personalized intervention plan, wherein the user interface is executed on at least one of the device or an external computing device communicatively coupled to the biometric analysis system.

11. The biometric analysis system of claim 10, wherein the at least one processor using the user interface is further configured to present the physiological conditions and the quantified emotional load profile using a color spectrum corresponding to entropy values associated with the physiological conditions or the quantified emotional load profile, wherein the color spectrum comprises a gradient ranging from lighter colors representing lower entropy values to darker colors representing higher entropy values.

12. The biometric analysis system of claim 10, wherein the at least one processor is further configured to present a three-dimensional representation of one or more organs or biological systems of the user, and to display one or more visual markers on the three-dimensional representation corresponding to locations of the physiological conditions or system-level imbalances.

13. The biometric analysis system of claim 1, wherein the analysis engine is further configured to compare spectral characteristics of the biological data with reference spectral data corresponding to healthy tissue and pathological conditions to determine a degree of similarity and identify a corresponding pathological tendency.

14. The biometric analysis system of claim 1, wherein the quantified emotional load profile comprises weighted indicators corresponding to stress, emotional reactivity, and behavioral patterns derived from the physiological signal variations.

15. The biometric analysis system of claim 1, wherein the analysis engine is configured to evaluate the physiological conditions and the quantified emotional load profile as a combined multi-domain model for determining the system-level imbalances.

16. The biometric analysis system of claim 1, wherein the one or more AI models are trained based on relationships between the physiological conditions, emotional load factors, and the reference parameters.

17. A biometric analysis device positioned in proximity to a user, comprising:
one or more diagnostic sensors integrated into the biometric analysis device, wherein the one or more diagnostic sensors are configured to transmit electromagnetic excitation signals within one or more predefined frequency ranges and receive corresponding electromagnetic response signals generated from interaction with the user to acquire biological data from the user, the biological data comprising at least bioelectrical activity, electromagnetic wave characteristics, biofrequency patterns, and physiological parameters associated with physiological activity of one or more tissues, one or more organs, and cellular activity; and
at least one processor communicatively coupled to the one or more diagnostic sensors and a memory storing instructions, wherein the at least one processor, upon executing the instructions, is configured to:
receive the biological data derived from the electromagnetic response signals and extract signal characteristics comprising frequency-domain features and waveform-based features representative of functional states of the user using signal transformation techniques;
convert, via a decoding module communicatively coupled to the at least one processor, the extracted signal characteristics into digital representations representing inferred physiological conditions and somatic imprints representing inferred functional states of the one or more tissues, the one or more organs or biological systems based on the extracted signal characteristics;
correlate, via a correlation module communicatively coupled to the at least one processor, the somatic imprints with emotional, neurological, and behavioral states of the user by applying one or more artificial intelligence (AI) models trained on reference data comprising physiological signal patterns and corresponding functional states, and generate a quantified emotional load profile computed from physiological signal variations corresponding to autonomic nervous system activities;
map, via a mapping module communicatively coupled to the at least one processor, the physiological conditions and the quantified emotional load profile to corresponding reference parameters within the reference data indicative of healthy and pathological states, by applying the one or more AI models;
evaluate, via an analysis engine communicatively coupled to the at least one processor, the physiological conditions and the quantified emotional load profile mapped to the corresponding reference parameters within the reference data to determine system-level imbalances;
generate, via a protocol generation module communicatively coupled to the at least one processor, a personalized intervention plan based on the determined system-level imbalances; and
iteratively update, via a feedback module communicatively coupled to the at least one processor, the personalized intervention plan based on subsequently acquired biological data to enable continuous recalibration of the physiological and emotional state of the user.

18. The biometric analysis device of claim 17, wherein the one or more diagnostic sensors comprise electromagnetic sensors configured to transmit and receive the electromagnetic excitation signals and the electromagnetic response signals to acquire the biofrequency patterns corresponding to the physiological activity of the one or more tissues or the one or more organs of the user, wherein the one or more diagnostic sensors comprise at least one of an electromagnetic transceiver, a biofrequency sensor, a resonance-based sensor, or a bioimpedance sensor.

19. A computer-implemented method for biometric analysis, comprising:
transmitting electromagnetic excitation signals within one or more predefined frequency ranges and receiving corresponding electromagnetic response signals generated from interaction with the user, via one or more diagnostic sensors integrated into a device configured to be positioned in proximity to a user, to acquire biological data from the user;
receiving, via at least one processor communicatively coupled to the one or more diagnostic sensors, the biological data derived from the electromagnetic response signals and extracting signal characteristics comprising frequency-domain features and waveform-based features representative of functional states of the user using signal transformation techniques;
converting, via a decoding module communicatively coupled to the at least one processor, the extracted signal characteristics into digital representations representing inferred physiological conditions and somatic imprints representing inferred functional states of the one or more tissues, the one or more organs or biological systems based on the extracted signal characteristics;
correlating, via a correlation module communicatively coupled to the at least one processor, the somatic imprints with emotional, neurological, and behavioral states of the user by applying one or more artificial intelligence (AI) models trained on reference datasets comprising physiological signal patterns and corresponding functional states, and generate a quantified emotional load profile computed from physiological signal variations corresponding to autonomic nervous system activity;
mapping, via a mapping module communicatively coupled to the at least one processor, the physiological conditions and the quantified emotional load profile to corresponding reference parameters within reference data indicative of healthy and pathological states, by applying the one or more AI models;
evaluating, via an analysis engine communicatively coupled to the at least one processor, the physiological conditions and the quantified emotional load profile mapped to the corresponding reference parameters against the reference data to determine system-level imbalances;
generating, via a protocol generation module communicatively coupled to the at least one processor, a personalized intervention plan based on the determined system-level imbalances; and
updating, via a feedback module communicatively coupled to the at least one processor, iteratively the personalized intervention plan based on subsequently acquired biological data to enable continuous recalibration of the physiological and emotional state of the user.

20. The method of claim 19, wherein the one or more diagnostic sensors comprise electromagnetic sensors configured to transmit and receive the electromagnetic excitation signals and the electromagnetic response signals to acquire the biofrequency patterns corresponding to the physiological activity of the one or more tissues or the one or more organs of the user, wherein the one or more diagnostic sensors comprise at least one of an electromagnetic transceiver, a biofrequency sensor, a resonance-based sensor, or a bioimpedance sensor.
